(19) United States
(12) Patent Application Publication
(10) Pub. No.: US 2026/0258397 A1
(43) Pub. Date: Sep. 3, 2026

(54) REAGENTS AND SYSTEMS FOR GENERATING BIOSENSORS

(71) Applicants: The Regents of the University of California, Oakland, CA (US); The Regents of the University of Colorado, a body corporate, Denver, CO (US)

(72) Inventor: Sean R. Cutler

(21) Appl. No.: 18/832,044

(22) PCT Filed: Jan. 20, 2023
(86) PCT No.: PCT/US2023/061024

§ 371 Date: Jul. 22, 2024

(51) Int. Cl. C12N 15/10; C07K 14/415; C12Q 1/42; C40B 40/10; G01N 21/64

(52) U.S. Cl. CPC C12N 15/1055; C07K 14/415; C12Q 1/42; C40B 40/10; G01N 21/6428; G01N 2021/6432; G01N 2333/916

(57) ABSTRACT

The disclosure relates to reagents and systems for generating a biosensor employing plant abscisic acid receptor polypeptide mutagenized in the ligand-binding pocket as a scaffold to detect small molecules. Here, we describe an approach for the rapid engineering of biosensors using a PYR1/PYL receptor polypeptide, a plant abscisic acid (ABA) receptor with a malleable ligand-binding pocket and'a requirement for ligand-induced heterodimerization, which facilitates the construction of sense-response functions.

### CROSS-REFERENCE TO RELATED APPLICATIONS

This application is a U.S. National Phase of International Application No. PCT/US2023/061024, filed Jan. 20, 2023, which claims benefit of priority to U.S. provisional application 63/301,402, filed Jan. 20, 2022, contents of which are herein incorporated by reference for all purposes.

### STATEMENT AS TO RIGHTS TO INVENTIONS MADE UNDER FEDERALLY SPONSORED RESEARCH AND DEVELOPMENT

This invention was made with government support under Contract No. HR001118C0137 awarded by the U.S. Department of Defense; Contract Nos. 2128287, 2128016, and 2128246 awarded by the National Science Foundation; and Contract No. 1R21DA053496 awarded by the National Institutes of Health. The government has certain rights in this invention.

### SEQUENCE LISTING

The instant application contains a Sequence Listing which has been submitted electronically in XML format and is hereby incorporated by reference in its entirety. Said XML copy, created on May 2, 2023, is named 081906-1370008-243810PC SL and is 181,000 bytes in size.

### BACKGROUND OF THE INVENTION

The ability to generate biosensors for user-defined molecules would substantially accelerate many areas of biotechnology. However, designing sensitive, specific, and portable biosensors remains a difficult problem in biotechnology. A number of protein scaffolds have been co-opted from nature or designed from scratch to develop sensors for user-defined molecules, including bacterial allosteric transcription factors ^{1,2}, G-protein coupled receptors ³, and computationally redesigned binding proteins ^{4,5,6}, amongst others. Each of these technologies have been successful in creating biosensors for a given application; however, they are limited by types of output signals available (e.g., transcriptional regulation).

Chemically-induced dimerization (CID) provides an appealing mechanism for coupling sensing to actuation; two proteins form a stable heterodimer only in the presence of a small molecule. Because they rely on a single protein-protein interaction, CID sensors can be used to build modular protein architectures that regulate transcription, control protein localization and degradation, as well as modulate cell signaling ⁷. However, in the CID systems typically exploited for sensing, ligand binding is shared between the binding partners, which necessitates redesign of the entire interface.

The plant abscisic acid (ABA) sensing system ^{8,9} functions through a naturally occurring CID mechanism ⁷ where ligand recognition by PYR1 leads to the formation of a stable PYR1-ligand-protein phosphatase (PP2C) complex that inhibits phosphatase activity. This system is unique, because ligand recognition occurs exclusively within PYR1, which simplifies the engineering of new CID modules. In addition, the phosphatase acts analogously to a co-receptor, because its binding to PYR1 lowers ligand off-rates and boosts apparent affinity up to ~100-fold ^{10,11}. Thus, micromolar PYR1-binders in isolation can function as nanomolar sensors in combination with the phosphatase.

It has additionally previously been shown that PYR1 can be repurposed to create an agrochemical receptor that functions in planta to modulate stress tolerance starting from a library of receptor variants created with single site saturation mutagenesis⁸.

### BRIEF SUMMARY OF THE INVENTION

Here, we exploit these beneficial traits using the Arabidopsis PYR1-PP2C system, PYR1-HAB1, to design new sensors for diverse chemical classes. Our data show that high-density mutagenesis of this scaffold enables rapid development of sense-response actuators portable to in vitro and in vivo control systems. Thus, in one aspect, the disclosure provides a library comprising a plurality of polypeptides, wherein each polypeptide contained in the plurality of polypeptides comprises a mutated PYR/PYL receptor polypeptide comprising I, L, R, V, or Y at position 62, relative to the corresponding position of SEQ ID NO: 1; and at least two amino acid substitutions, relative to the corresponding position of SEQ ID NO:1, selected from positions 59, 83, 89, 92, 94, 108, 117, 120, 122, 141, 159, 160, and 163, wherein the at least two amino acid substitutions differ among members of the library and are independently selected from A, D, E, F, G, H, I, K, L, M, N, Q, R, S, T, V, W and Y. In some embodiments, each polypeptide further comprises at least one of the following: I, L, M, R, T, V, or Y at position 81; A, F, G, I, L, M. V, W, or Y at position 87; or A, F, I, K, L, M R, T, V, or W at position 110. In some embodiments, each polypeptide comprises I, L, M, R, T, V, or Y at position 81; A, F, G, I, L, M, V, W, or Y at position 87; and A, F, I, K, L, M R, T, V, and W at position 110. In some embodiments, at least one, at least two, at least three, or all four of positions 62, 81, 87, and 110 of each polypeptide are substituted relative to the corresponding position of SEQ ID NO:1. In some embodiments, the plurality of polypeptides comprises polypeptides comprising at least three amino acid substitutions, relative to SEQ ID NO: 1, selected from positions 59, 83, 89, 92, 94, 108, 117, 120, 122, 141, 159, 160, and 163; and wherein the substitutions are independently selected from A, D, E, F, G, H, I, K, L, M, N, Q, R, S, T, V, W and Y. In some embodiments, position 62 is I; position 81 is V; position 87 is L, or position 110 is 1. In some embodiments, each polypeptide comprises 162, V81, L87, and 1110. In some embodiments, a mutated PYR/PYL receptor has at least 60% identity to SEQ ID NO:1. In some embodiments, a mutated PYR/PYL polypeptide has at least 60% identity to SEQ ID NO:2. In some embodiments, the library is a display library or a yeast two-hybrid library. In some embodiments, the library is immobilized to a solid support. In some embodiments, each of the PYR1 receptor polypeptides further comprises a detection reagent. In some embodiments, the detection agent is a member of a binding complex or an enzymatic complementation reagent.

In a further aspect, the disclosure provides a system for detecting a small molecule, comprising a library of any one of claims 1 to 11, and a type 2 protein phosphatase (PP2C) polypeptide. In some embodiments, the PP2C polypeptide is a HAB1 polypeptide. In some embodiments, the HAB1 polypeptide is an N-terminus deleted HAB1 polypeptide. In some embodiments, the N-terminus deleted HAB1 polypeptide is thermostable. In some embodiments, the N-terminus deleted HAB1 polypeptide has at least 95% identity to SEQ ID NO: 3 and comprises the amino acid residues at positions 393, 406, 411, 416, 455, 467, 481, 494, 499, 549, 565, 584, 629, 646, and 698 as set forth in SEQ ID NO:3. In some embodiments, the N-terminus deleted HAB1 polypeptide comprises the amino acid sequence of SEQ ID NO:3. In some embodiments, the PP2C polypeptide is labeled with a detection reagent. In some embodiments, members of the library are labeled with a first detection reagent and the PP2C polypeptide is labeled with a second detection reagent that interacts with the first detection reagent, wherein a signal is generated when a member of the library binds the small molecule and the first detection reagent interacts with the second detection reagent. In some embodiments, the first detection reagent specifically binds to the second detection reagent. In some embodiments, the first detection reagent is an antibody, ligand, or aptamer that specifically binds to the second detection moiety. In some embodiments, the second detection reagent is an antibody, ligand, or aptamer that specifically binds to the first detection moiety. In some embodiments, the first detection reagent and the second detection reagent are complementing fragments of an enzyme. In some embodiments, the enzyme is luciferase. In some embodiments, the first detection reagent and the second detection reagent are fluorescent moieties, or one of the first detection reagents is a fluorescent moiety and the second is a quenching moiety that quenches the fluorescent moiety. In some embodiments, the first detection reagent and the second detection reagent are oligonucleotides, the proximity assay is a proximity extension assay or proximity ligation assay.

In a further aspect, the disclosure provides a method of identifying a biosensor for a small molecule of interest, comprising contacting the system as described herein, e.g., in the preceding paragraph, with the small molecule of interest, and identifying that a mutated PYR/PYL contained in the plurality of polypeptides binds to the small molecule of interest.

In another aspect, the disclosure provides a HAB1 polypeptide comprising an N-terminus deletion, wherein the HAB1 polypeptide has at least 95% identity to SEQ ID NO:3 and comprises the amino acid residues at positions 393, 406, 411, 416, 455, 467, 481, 494, 499, 549, 565, 584, 629, 646, and 698 as set forth in SEQ ID NO:3. In some embodiments, the HAB1 polypeptide comprises the amino acid sequence of SEQ ID NO:3.

In an additional aspect, the disclosure provides a cannabinoid-binding polypeptide comprising an amino acid sequence having at least 95% identity to any one of SEQ ID NOS: 6-22, wherein the polypeptide comprises each of the highlighted amino acid residues in the corresponding sequence. In some embodiments, the polypeptide comprises the amino acid sequence of any one of SEQ ID NOS: 6-22.

In a further aspect, the disclosure provides an organophosphate-binding polypeptide comprising an amino acid sequence having at least 95% identity to any one of SEQ ID NOS: 23-30, wherein the polypeptide comprises each of the highlighted amino acid residues in the corresponding sequence. In some embodiments, the polypeptide comprises the amino acid sequence of any one of SEQ ID NOS: 23-30.

The disclosure also provides a cannabinoid and organophosphate-binding polypeptide comprising an amino acid sequence having at least 95% identity to SEQ ID NO: 31 or 32, wherein the polypeptide comprises each of the highlighted amino acid residues in the corresponding sequence. In some embodiments, the polypeptide comprises the amino acid sequence of SEQ ID NO:31 or 32.

In addition, the disclosure provides a biosensor polypeptide comprising an amino acid sequence having at least 95% identity to any one of SEQ ID NOS: 33-37, wherein the polypeptide comprises each of the bolded amino acid residues in the corresponding sequence. In some embodiments, the biosensor polypeptide comprises the amino acid sequence of any one of SEQ ID NOS: 33-37

### BRIEF DESCRIPTION OF THE DRAWINGS

FIG. 1a-c: Protein structure-guided design of high-affinity PYR1-based cannabinoid sensors. a. The 19 side chains of residues in PYR1's binding pocket targeted for double site mutagenesis (DSM) are shown along with ABA (yellow) and HAB1's W385 “lock” residue and water network (3QN1). b. Sensor evolution pipeline. The PYR1 library was constructed by nicking mutagenesis ^{9,12} in two sub-pools, one using single mutant oligos and another using double mutant oligo pools. The combined pools were screened for new sensors using Y2H growth selections in the presence of a ligand of interest. c. Representative screen results. The DSM library was screened for mutants that respond to the synthetic cannabinoid JWH-015 yielding five hits that were subsequently optimized by two rounds of DNA shuffling to yield PYR1^{JWA-015}, which harbors 4 mutations. The Y2H staining data show different receptor responses to JWH-015 by beta-galactosidase activity.

FIG. 2a-e: Sequence and structural basis of ligand recognition. a. Sequence diversity of cannabinoid receptor ligand binding pocket residues. The minimal ligand concentrations required for Y2H signal generation indicated at right (see FIG. 5 for full data, including mutations outside the pocket). The heatmap shows ligands clustered by Tanimoto scores, calculated in ChemMine ¹⁶. b. Representative optimized sensor Y2H beta-galactosidase responses to the ligands indicated. c-e. Structural basis for cannabinoid recognition. c. WIN is colored yellow, and key ligand-contacting residues are indicated with dashes. The Trp lock water network that stabilizes binding is shown at top. d. Relief of steric clash by the evolved receptor. e. Structural poses of WIN in PYL2-bound (top) and CB2-bound (bottom, 6PT0) structures.

FIG. 3a-e: PYR1-based sensors are portable to diverse CID-based output systems demonstrated with PYR1^{4F} and PYR1^{WIN}. (a) Phosphatase inhibition. Ligand-dependent inhibition of ΔN-HAB1 phosphatase activity by recombinant receptors using a fluorogenic substrate. Inhibition expressed relative to mock controls (n=3). (b) Gene activation. Ligand-induced gene activation in S. cerevisiae using an engineered Z4_{DBD}-PYR1/VP64_{AD}-ΔN-HAB1 genetic circuit. Whole cell fluorescence generated from an integrated Z4₄-CYC1_{core}-GFP-CYC1t reporter is shown (12 h post ligand addition; n=3). (c) Split luciferase complementation. Addition of ligand results in luminance from Luc^{N}-PYR1/Luc^{C}-ΔN-HAB1 (n=3). (d) PYR1 ELISA-like immunoassays. Immobilized receptors recruit biotinylated ΔN-HAB1^{T+} in response to ligand and colorimetric signal is generated by a secondary streptavidin HRP conjugate; n=2, A₄₅₀ background subtracted. 95% c.i. shown on fits in b, c, & d. (e) Receptor cross-reactivity evaluation in PYR1 ELISAs. The cannabinoids shown were assayed for signal generation at 2 μM. “+CNTRL” is PYR1^{M} tested with 2 μM ABA.

FIG. 4a-e: Facile development of potent, selective, and portable organophosphate sensors. (a.) Summary of biosensor screening results for a panel of 10 organophosphates. The compounds screened are clustered by similarity (blue=more similar) using a distance matrix of pairwise Tanimoto similarity scores, calculated in ChemMine 16. The molecules that yielded hits are shown in bold type; the minimal ligand concentrations required for Y2H signal generation for optimized receptors (see methods) are indicated (see FIG. 13a-d for additional details). (b.) The optimized PYR1^{DIAZI} and PYR1^{PIRI} are high-affinity sensors. Optimized receptors were tested for responses to nM concentrations of diazinon and pirimiphos-methyl, respectively, as evidenced by Y2H assays and receptor-mediated inhibition of HAB1 phosphatase activity in vitro. PYR1^{DIAZI} (EC₅₀=36 nM [32, 40]); PYR1^{PIRI} (EC₅₀=58 nM [50, 67]). Wild type PYR1 was used as a control (gray lines). (c.) PYR1-derived receptors are portable. PYR1^{DIAZI} and PYR1^{PIRI} were tested in a protein fragment complementation system based on split luciferase reconstitution with Luc^{N}-PYR1/Luc^{C}-HAB1 fusions in yeast (PYR1^{DIAZI}, EC₅₀=24 nM [12, 50]; PYR1^{PIRI}, EC₅₀=19 nM [undef, 29]). (d., e.) PYR1^{DIAZI} and PYR1^{PIRI} are selective for their evolved target ligands. (d.) Y2H (top) and in vitro phosphatase inhibition assays (bottom) were used to profile receptor responses; the receptors no longer bind the native ligand ABA. Pirimiphos-methyl and diazinon were tested 20 nM, ABA, tested at 5000 nM in Y2H assays. (e.) Characterization of receptor selectivity using a Z4-PYR1/VP64-HAB1 gene activation circuit in the presence of the activating ligands shown (see Table. S7 for quantitative analyses of EC_{50S} values). In all cases, the symbol represents the mean, and error bars shown are I s.d. and may be smaller than the symbol.

FIG. 5a-c: Chemical diversity of natural and synthetic cannabinoids sensed by engineered PYR1 sensors. (a) Y2H data of sensor hits. (b) Chemical structures of compounds for which at least one PYR1-based biosensor was identified. (c) Chemical structures of compounds for which no PYR1-based biosensor was identified.

FIG. 6: Sensor cross-reactivity of PYR1-based sensors for synthetic cannabinoids.

FIG. 7: Sensitivity of the PYR1 and PYL2 variants of the WIN 55,212-2 and 4F-MDMB-BINACA sensors. The WIN 55,212-2 sensors are selectivity toward the (+)-stereoisomer; we note that a subset of the staining data shown in this figure for (+)-WIN 55,212-2 was used in the main text FIG. 2b to illustrate the PYR1^{WIN} sensor response.

FIG. 8: Assessment of stabilized, inactivated 6×His-MBP-ΔN-HAB1 variants. Experiments were repeated on two separate days using independently generated batches of protein. Error bars indicate 1 SD of 3 measurements. Sets of mutations for each variant are noted in Table 7. The first row shows Michaelis-Menten enzyme kinetics for 6×His-MBP-ΔN-HAB1 variants using p-NPP. The second row shows determination of apparent Tm for 6×His-MBP-ΔN-HAB1 variants using YSD of PYR1^{M}, in the presence of 500 nM ABA and 20 nM 6×His-MBP-ΔN-HAB1 variant. The third row shows YSD titrations of noted HAB1 variant against PYR1^{M} in the presence of 500 nM ABA. Fluorescence here refers to streptavidin-phycoerythrin.

FIG. 9a-f: Electron density of WIN 55,212-2 in the PYL2^{WIN} ligand binding pocket. Several rounds of structural refinement (a) were carried out before modeling WIN 55,212-2 into the ligand binding pocket's unbiased electron density (b+c); a real-space correlation coefficient of 0.967 calculated between the unbiased electron density and (+)-WIN 55,212-2 indicates agreement between the model and observed electron density. Ligplot illustrating hydrophobic and water-mediated contacts between WIN 55212-2 and the engineered PYL2^{WIN} receptor (d) in comparison to the hydrophobic contacts that stabile WIN 55212-2 binding to the human cannabinoid receptor CB2 (e) (PDB 6PT0). The binding poses (conformations) of WIN 55212-2 also differ substantially between the two structures (see main FIG. 2e). (f) Water-mediated hydrogen bonds connecting PYL2^{WIN} and ligand are realized through K59Q, which rearranges the extensive hydrogen bond network at the base of the pocket.

FIG. 10a-b: The structure of ΔN-HAB1^{T+} is unchanged compared to ΔN-HAB1^{WT} (a) Alignment of ΔN-HAB1^{WT} (PDBID: 3QN1) to ΔN-HAB1^{T+}. All-atom RMSD is 0.85 Å. The tryptophan latch W385 and two mutated active site residues R199A/D204A are shown as sticks. (b) Superimposed active sites of the two proteins. Individual rotamers are essentially unchanged.

FIG. 11a-c: Reconstitution and optimization of the PYR1/HAB1 CID system in a plate-based ELISA. (a) Symbols represent empirical titration curves of ABA with increasing concentrations of ΔN-HAB1^{T+} in the binding assay with immobilized PYR1. Lines indicate global best fit based upon the empirical data. Concentrations of HAB used are indicated as follows: purple circles are 1000 nM, blue squares are 333 nM, gold up-pointing triangles are 111 nM, orange diamonds are 37 nM, green starbursts are 12.3 nM, and brown down-triangles are 4.1 nM. The structure of ABA is shown. (b) Effective Ko values for the PYR1/ABA/ΔN-HAB1^{T+} system in a plate-based ELISA assay, using the data from Panel a. Data points indicate three independent biological replicates performed on separate days. Note the log scale. (c) Background signal generated from interaction of PYR1 and ΔN-HAB1^{T+} in the absence of ABA, shown from the same biological replicates used for panels a. and b. Note the linear scale. Experiments were repeated on three separate days.

FIG. 12a-b: Reconstitution of the PYR/HAB based CID system in a plate-based ELISA. Experiments were repeated on two separate days and data points indicate values for both technical replicates within one of the independent days. (a) Titration curve of a select subset of cannabinoids with immobilized PYR^{WIN-E} and 1 μM ΔN-HAB1^{T+}. Ligands are indicated as follows: blue circles are WIN55,212, green squares are JWH-015, orange diamonds are JWH-072, and purple triangles are 4F-MDMB-BUTINACA. Data shown are “No ligand control” subtracted. Structure of the target ligand, WIN55,212 is shown. (b) Titration curve of a select subset of cannabinoids with immobilized PYR4P-E and 1 AM ΔN-HAB1^{T+}. Ligands are indicated as follows: blue circles are WIN55,212, green squares are JWH-015, orange diamonds are JWH-072, and purple triangles are 4F-MDMB-BUTINACA. Data shown are “No ligand control” subtracted. Structure of the target ligand, 4F-MDMB-BUTINACA is shown.

FIG. 13a-d: Initial hits and optimization of organophosphate PYR1-based sensors. (a) Mutations and Y2H data of the initial organophosphate hits. The library used for screening is indicated on the left. (b) Binding pocket mutations of optimized diazinon and pirimiphos receptors. Binding pocket mutations in the primary hit is also shown. (c) Screened organophosphates and the libraries used for initial screening and optimization. See methods for details of the optimization libraries indicated as OP-S(1-4). (d) Chemical structures of the screened organophosphates.

FIG. 14: Minimum ΔΔG in Rosetta Energy Units (REU) predicted by Rosetta for each candidate mutation in the library. Mutations to cysteine or proline were excluded.

### DETAILED DESCRIPTION OF THE INVENTION

### Terminology

The term “PYR/PYL receptor polypeptide” refers to a protein characterized in part by the presence of one or more or all of a polyketide cyclase domain 2 (PF10604), a polyketide cyclase domain 1 (PF03364), and a Bet V I domain (PF03364), which in wild-type form mediates abscisic acid (ABA) and ABA analog signaling. A wide variety of PYR/PYL receptor polypeptide sequences are known in the art. In some embodiments, a PYR/PYL receptor polypeptide comprises a polypeptide that is at least 60%, 65%, or 70% identical to PYR1 (SEQ ID NO:1); or at least 75%, 80% or 85% identical to SEQ ID NO: 1; and has at least one mutation in the binding pocket as described herein. In some embodiments, a mutant PYR/PYL receptor polypeptide comprises a polypeptide that is at least 90% identical, or at least 95% identical, to PYR1 (SEQ ID NO:1) and has one or mutations in the binding pocket as described herein. In some embodiments a PYR/PYL receptor polypeptide comprises a polypeptide that is at least 60%, 65%, or 70% identical to PYL2 (SEQ ID NO:2), or at least 75%, 80% or 85% identical to SEQ ID NO:2; and has at least one mutations in the binding pocket as described herein. In some embodiments, a PYR/PYL receptor polypeptide comprises a polypeptide that is at least 90% identical, or at least 95% identical, to PYL2 (SEQ ID NO:2) and has one or more mutations in the binding pocket as described herein.

A “wild-type PYR/PYL polypeptide” refers to a naturally occurring PYR/PYL receptor polypeptide that mediates abscisic acid (ABA) and ABA analog signaling.

A “mutated PYR/PYL polypeptide” or “variant PYR/PYL polypeptide refers to a PYR/PYL receptor polypeptide that is a variant from a naturally-occurring (i.e., wild-type) PYR/PYL receptor polypeptide. As described herein, a mutated PYR/PYL receptor polypeptide comprises at least one mutation in the binding pocket as described herein. In some embodiments, the polypeptide comprises one, two, three, four, or more amino acid substitutions in the binding pocket relative to a corresponding wild-type PYR/PYL receptor polypeptide as described herein. In this context, a “mutated” polypeptide can be generated by any method for generating non-wild type nucleotide sequences.

An “amino acid substitution” refers to replacing the naturally occurring amino acid residue in a given position (e.g., the naturally occurring amino acid residue that occurs in a wild-type PYR/PYL receptor polypeptide) with an amino acid residue other than the naturally-occurring residue.

An amino acid residue “corresponding to an amino acid residue [X] in [specified sequence],” or an amino acid substitution “corresponding to an amino acid substitution [X] in [specified sequence]” refers to an amino acid in a polypeptide of interest that aligns with the equivalent amino acid of a specified sequence. Generally, as described herein, the amino acid corresponding to a position of a specified PYR/PYL receptor polypeptide sequence can be determined using an alignment algorithm such as BLAST. In some embodiments of the present invention, “correspondence” of amino acid positions is determined by aligning to a region of the PYR1 polypeptide comprising SEQ ID NO:1, as discussed further herein.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). For purposes of this application, percent identity of protein and nucleic acid sequences are determined using BLAST algorithms.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived. As explained herein, these substantially identical variants are specifically covered by reference to a specific nucleic acid sequence.

The term “host cell” refers to any cell capable of replicating and/or transcribing and/or translating a heterologous polynucleotide. Thus, a “host cell” refers to any prokaryotic cell (including but not limited to E. coli) or eukaryotic cell (including but not limited to yeast cells, mammalian cells, avian cells, amphibian cells, plant cells, fish cells, and insect cells), whether located in vitro or in vivo. For example, host cells may be located in a transgenic animal or transgenic plant. prokaryotic cell (including but not limited to E. coli) or eukaryotic cells (including but not limited to yeast cells, mammalian cells, avian cells, amphibian cells, plant cells, fish cells, and insect cells). Host cells can be for example, transformed with the heterologous polynucleotide.

### PYR/PYL Receptor Polypeptide Libraries

Libraries to screen for biosensor for small molecule compounds as described herein comprise mutated PYR/PYL receptor polypeptides having substitutions, relative to a wildytpe PYR/PYL polypeptide, e.g., SEQ ID NO:1, or SEQ ID NO:2. The binding pocket of PYR1 and homologs comprises a conserved START-domain ligand-binding pocket flanked by two loops called the “gate” and the “latch” (Melcher, K. et al., Nature 462 (2009)). A compound binds to PYR/PYL receptor protein at the ligand-binding pocket and binding induces closure of the loops. In wild-type PYR/PYL receptor proteins, residues comprising the ligand-binding pocket are those residues with side chains that are within 4 angstroms of ABA or the water molecules that accompany ABA when ABA binds in the pocket of the PYR/PYL receptor protein. For purposes of the present disclosure, the 19 residues lining the PYR1 (SEQ ID NO:1) binding pocket that can be mutated to generate biosensors residues for generating biosensors are: K59, 162, V81, V83, L87, A89, S92, E94, F108, 1110, L117, Y120, S122, E141, F159, A160, V163, V164, and N167. In some embodiments, the library comprises mutations at at least one, two, three, four, five, or more positions of the binding pocket residues corresponding to positions K59, 162, V81, V83, L87, A89, S92, E94, F108, I110, L117, Y120, S122, E141, F159, A160, V163, V164, and N167 of SEQ ID NO:1. In some embodiments, the library comprise mutations at at least one, two, three, four, five, or more positions of the binding pocket residues corresponding to positions K64, V67, V85, V87, L91, A93, S96, E98, F112, V114, L121, Y124, S126, E147, F166, V167, V170, V171, and N174 of SEQ ID NO:2. For convenience, binding pocket residue positions are described below using SEQ ID NO: 1 as a reference sequence. It is understood that the corresponding positions of other PYR/PYL polypeptides can be readily determined by one of skill in the art. For example, corresponding binding pocket positions of SEQ ID NO:2 are: YL2, K64, V67, V85, V87, L91, A93, S96, E98, F112, V114, L121, Y124, S126, E147, F166, V167, V170, V171, and N174.

In some embodiments, the library comprises variant PYR/PYL polypeptides of comprising I at position 62 of SEQ ID NO: 1 and at least two additional mutation at an unconstrained position, i.e., the position can have any amino acid substitution other than Cys or Pro at a position corresponding to position 59, 83, 89, 92, 94, 108, 117, 120, 122, 141, 159, 160, or 163 of SEQ ID NO:1. In some embodiments, a variant polypeptide comprises L. R. V, or Y at position 62 and one or more, or two or more, or at least three or more, substitutions at positions 59, 83, 89, 92, 94, 108, 117, 120, 122, 141, 159, 160, or 163 of SEQ ID NO:1.

In some embodiments, the library comprises variant PYR/PYL polypeptides comprising I, L, R, V or Y at position 62; and a mutation at at least one position corresponding to position 81, 87, or 110 of SEQ ID NO:1, wherein the residue at position 81 is I, L, M. R, T, or Y; position 87 is A, F, G, I, M, V, W or Y and/or position 110 is A, F, K, L, M, R, T, V, or W.

In some embodiments, the library comprises variant PYR1 polypeptides comprising I, L. R, V or Y at position 62, I, L, M, R, T, V or Y at position 82; A, F, G, I, L, M, V, W or Y at position 87 and A, F, I, K, L, M, R, T, V or W at position 110; and at least one additional mutation, or at least two additional substitutions, at an unconstrained position corresponding to position 59, 83, 89, 92, 94, 108, 117, 120, 122, 141, 159, 160, or 163 of SEQ ID NO:1, in which any amino acid other than Cys or Pro is substituted relative to the amino acid residue at the corresponding position of SEQ ID NO:1. In some embodiments, the library comprises variant PYR1 polypeptides comprising I, L, R, V or Y at position 62, I, L, M, R, T, V or Y at position 82; A, F, G, I, L, M, V, W or Y at position 87 and A, F, I, K, L, M, R, T, V or W at position 110; and at least three additional substitutions at an unconstrained position corresponding to position 59, 83, 89, 92, 94, 108, 117, 120, 122, 141, 159, 160, or 163 of SEQ ID NO:1, in which any amino acid other than Cys or Pro is substituted relative to the amino acid residue at the corresponding position of SEQ ID NO:1

In some embodiments, a mutated PYR/PYL receptor polypeptide having multiple mutations in the binding pocket, e.g., at least three or more mutations in the binding pocket as described herein has at least 80%, 85%, 90%, 91%, 92%, 93%, 94%, 95%, 96%, 97%, 98%, or 99% identical to) SEQ ID NO:1. In some embodiments, the PYR/PYL polypeptide has at least 90% identity to SEQ ID NO:1.

### I. Methods of Making Mutated PYR/PYL Receptor Polypeptides

Embodiments of the present invention provide for methods of generating libraries of mutated PYR/PYL receptor polypeptides for the identification of biosensor molecules that detect a small molecule of interest. In some embodiments the method comprises mutagenizing a wild-type PYR/PYL receptor polypeptide to introduce mutations into the binding pocket residues described herein, contacting members of the library with a small molecule of interest, and determining whether small molecules binds to the mutated PYR/PYL receptor polypeptide. Mutated PYR/PYL receptor polypeptides can be constructed by mutating the DNA sequences that encode the corresponding wild-type PYR/PYL receptor polypeptide or a corresponding variant from which the mutant PYR/PYL receptor polypeptide of the invention is derived). Mutagenesis can be performed using site-directed mutagenesis or any technique to introduce mutations into the desired binding pocket residues. In some embodiments mutations are introduced at binding pocket mutations as described herein that are separated by at least eight amino acids to generate a library. In some embodiments, such a library is combined with another library in which binding pocket mutations are introduced at positions that are separated by fewer than eight amino acids. Nucleic acid molecules encoding the wild-type PYR/PYL receptor polypeptide can be mutated by a variety of polymerase chain reaction (PCR) techniques well-known to one of ordinary skill in the art. (See, e.g., PCR Strategies (M. A. Innis, D. H. Gelfand, and J. J. Sninsky eds., 1995, Academic Press, San Diego, CA) at Chapter 14; PCR Protocols: A Guide to Methods and Applications (M. A. Innis, D. H. Gelfand, J. J. Sninsky, and T. J. White eds., Academic Press, NY, 1990).

By way of non-limiting example, mutagenesis may be accomplished as described in the Examples section. Following amplification, the reaction products are cloned into a suitable vector to construct a library, which can then be transformed into suitable cells (e.g., yeast cells) for subsequent screening (e.g., via a two-hybrid screen). In some embodiments, the library may be a display library, e.g., a yeast display library or phage display library.

In some embodiments, an initial screen for binding to a small molecule of interest is performed, e.g., using a yeast two-hybrid screen and followed by further mutagenesis, such as DNA shuffling). Briefly, a shuffled mutant library is generated through DNA shuffling using in vitro homologous recombination by random fragmentation of a parent DNA followed by reassembly using PCR, resulting in randomly introduced point mutations. Methods of performing DNA shuffling are known in the art (see, e.g., Stebel, S. C. et al., Methods Mol Biol 352:167-190 (2007)).

Optionally, multiple rounds of mutagenesis may be performed. Thus, in some embodiments, PYR/PYL mutants may be pooled and used as templates for later rounds of mutagenesis.

Screening of PYR/PYL libraries is performed based on a naturally occurring CID mechanism ⁷ where ligand recognition by PYR1 leads to the formation of a stable PYR1-ligand-protein phosphatase (PP2C).

### II. Screening to Identify a Small Molecule Biosensor

Embodiments of the present invention also provide for methods and systems for screening small molecules to determine binding to a mutated PYR/PYL receptor polypeptide having mutations in the binding pocket as described herein, in which the methods/systems employ a type 2 protein phosphatase (PP2C) polypeptide component where ligand recognition by the PYR/PYL polypeptide leads to the formation of a stable PYR/PYL-ligand-PP2C complex).

In some embodiments, the PP2C polypeptide is HAB1 polypeptide, comprising an amino acid sequence having at least 75%, 80%, or 85% identity to SEQ ID NO:3 or SEQ ID NO: 5. In some embodiments, the PP2C polypeptide is a HAB1 polypeptide comprising an amino acid sequence having at least 90% or at least 95% identity to SEQ ID NO:3 or SEQ ID NO: 5. In some embodiments, the HAB1 polypeptide comprises a deletion at the N-terminus, such that the HAB1 polypeptide comprises a catalytic domain comprising the amino acid sequence of SEQ ID NO:3 or the catalytic domain of SEQ ID NO:5. In some embodiments, the HAB1 comprises a thermostable variant of the catalytic domain of SEQ ID NO:5. In some embodiments, the thermostable variant comprises a mutation at two or more residues selected from positions 393, 406, 411, 416, 455, 467, 481, 494, 499, 549, 565, 584, 629, 646, and 698 of SEQ ID NO:5. In some embodiments, the thermostable variant comprises a mutation five or more, or ten or more, of positions 393, 406, 411, 416, 455, 467, 481, 494, 499, 549, 565, 584, 629, 646, and 698 of SEQ ID NO:5. In some embodiments, the thermostable variant comprises the residues at positions 393, 406, 411, 416, 455, 467, 481, 494, 499, 549, 565, 584, 629, 646, and 698 that are depicted in SEQ ID NO:3.

Screening can take place using isolated, purified or partially purified reagents. In some embodiments, purified or partially purified PYR/PYL polypeptide can be used.

In some embodiments, compounds of less than 1000 Daltons in size are screened. In some embodiments, small molecules that fall within the Lipinski's rule of five (i.e., a molecule with a molecular mass less than 500 Da, no more than 5 hydrogen bond donors, no more than 10 hydrogen bond acceptors, and an octanol-water partition coefficient log P not greater than 5) are screened.

Binding assays can involve contacting a PYR/PYL receptor polypeptide library as described herein and a PP2C polypeptide with one or more chemical agents and allowing sufficient time for the proteins and chemical agents to form a binding complex. In some embodiments, the polypeptides of the PYR/PYL library and/or the PP2C polypeptide are labeled with a detection moiety. In some embodiments, both the PYR/PYL library and PP2C polypeptides are labeled with detection moieties that interact to generate a signal when a dimer is formed between a PYR/PYL and PP2C polypeptide upon binding to a small molecule. In some embodiments, the detection moieties are members of an enzyme complementation system, such as a luciferase enzyme fragment complementation assay, a beta-lactamase fragment complementation assay, a dihydrofolate reductase complementation assay, a Gal4 transcription factor complementation assay, a beta galactosidase complementation assay, a horseradish peroxidase complementation assay, and others.

In some embodiments, the PYR/PYL polypeptides are labeled with a binding moiety that interacts with a binding partner that is used to label the PP2C polypeptide. In some embodiments, the binding partner are antibodies, aptamers, or ligands that bind to a target protein.

In some embodiments, the PYR/PYL polypeptides and PP2C polypeptides are labeled with a fluorescent moiety and quencher. In some embodiments, the polypeptides are labeled with oligonucleotides, e.g., components of a proximity extension assay or proximity ligations assay.

In some embodiments, binding complexes formed can be detected using assays that include, but are not limited to, immunoassay analogous to ELISA assays, NMR techniques, displacement of labeled substrates (e.g., labeled agrochemical) or other assays. The PYR/PYL polypeptide protein utilized in such assays can be naturally expressed, cloned or synthesized.

Agents and mutated PYR/PYL receptor polypeptides that are initially identified by any of the foregoing screening methods can be further tested to validate the binding activity of the mutated PYR/PYL receptor polypeptide.

### EXAMPLES

These examples provide illustrative data demonstrating a platform for the rapid isolation of biosensors using the plant abscisic acid (ABA) receptor PYR1 (Pyrabactin Resistance 1), which has a malleable ligand-binding pocket and uses a ligand-induced dimerization mechanism that facilitates the construction of sense-response functions. The examples further provide validation data by evolving 18 sensors with nM to μM sensitivities for a range of small molecules, including structurally diverse cannabinoids and organophosphates. An X-ray crystallographic structure provides additional insight into the basis for new ligand recognition by an evolved cannabinoid receptor. The data further illustrate that PYR1-derived receptors are readily ported to various ligand-responsive outputs, including ELISA-like assays, luminescence by protein-fragment complementation, and transcriptional circuits, all with pM to nM sensitivity

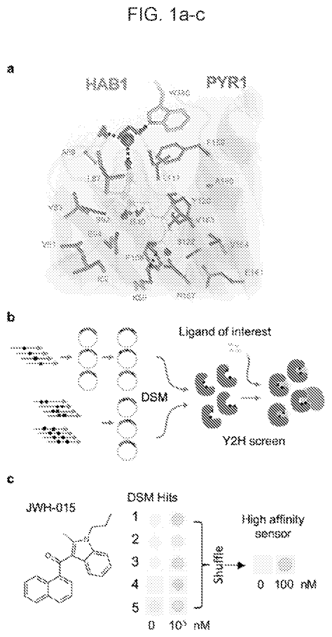

We reasoned that a structure-guided approach would enable the design of a larger, more diverse double mutant library biased towards stable mutations in the binding pocket and ultimately enable broader ligand recognition. Using the Rosetta protein design software we first determined the stability of all possible mutations of 19 residues lining PYR1's binding pocket (FIG. 1a). Based on this analysis, 15 residues were allowed to mutate to all amino acids except cysteine or proline and four positions were restricted to smaller amino acid subsets (Table 1). This computational design step condensed the double mutant library from a theoretical maximum size of 68,400 to 42,743 putatively stable double mutants. 6,291 of the desired mutants involve residues too close to one another for construction using single mutant oligonucleotides (i.e., within eight residues) and were, therefore, constructed by nicking mutagenesis using a pool of double mutant oligos (FIG. 1b) 9. The remaining 36,452 mutants were constructed using a pool of 301 single mutant oligos in two sequential rounds of single-site nicking mutagenesis 12. These two pools were then combined into a single double site mutant (DSM) pocket library for subsequent screening. The DSM library was deep-sequenced and determined to possess >99.8% of the desired double mutants (Table 2).

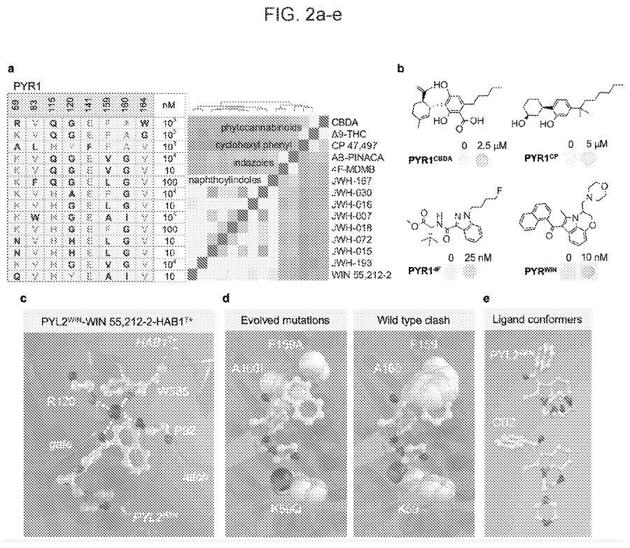

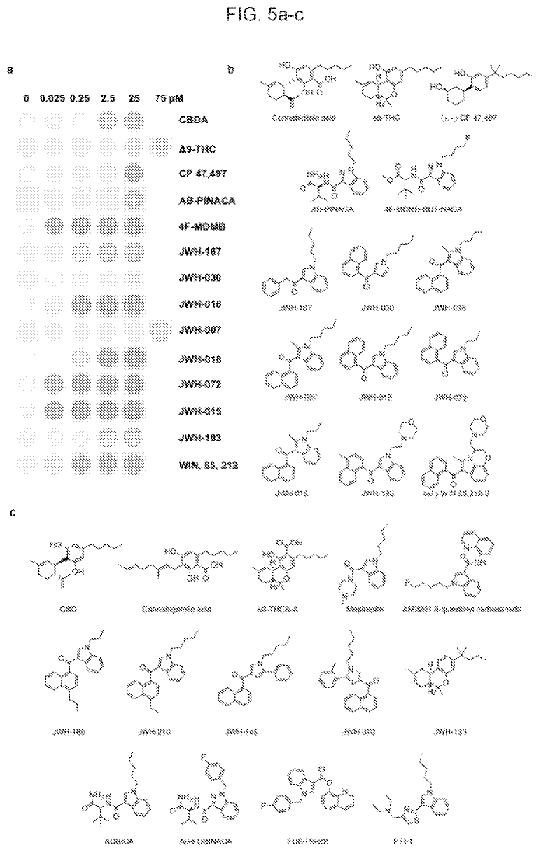

With the improved library in hand, we set out to test its efficiency in a number of screens for new biosensors. We first focused on developing cannabinoid sensors, in part to develop diagnostic reagents for synthetic cannabinoid mimics sold in products like “Spice”, which have caused many hospitalizations ¹³ and deaths ^{14,15}. We screened for PYR1 mutants responsive to any one of a panel of 28 cannabinoids, screening for mutants responsive to 30 μM of each test chemical (see FIG. 5b-c for chemical structures). Selections were accomplished in a yeast two-hybrid strain where expression of URA3 rescues uracil auxotrophy via PYR1 binding to HAB1. Prior to selection, mutations that produced ligand-independent URA3 activation were removed by a negative selection in the presence of 5-FOA. Our initial positive selections identified double mutants responsive to JWH-015 (FIG. 1c) and five other naphthoylindoles, as well as cannabicyclohexanol (CP 47,497), a different chemical scaffold and one of the active ingredients in “Spice”; this demonstrates that our library can yield sensors in a single screening step. Additional sensors were acquired by iteratively screening diversified cannabinoid-biased sub-libraries that were created by shuffling hit receptors against both the parental DSM and previous SSM libraries. Ultimately, these efforts identified 12 unique PYR1-derived cannabinoid receptors that recognize 14 compounds, including sensors for CBDA, 49-THC, and 4F-MDMB-BUTINACA (4F-MDMB), from a total of 28 cannabinoids screened (FIG. 2a,b, FIG. 5a-c). Overall, mutations in nine out of the 19 residue positions targeted in the parental library were obtained in the cannabinoid receptors (K59, V81, V83, L87, A89, Y120, F159, A160, V164), along with two additional sites (H115 and M158) present in the SSM library used for DNA shuffling and affinity maturation. We note that in two cases our selections converged on the same sequences; receptors responsive to JWH-072 and JWH-015, closely related naphthoylindoles that differ by only a single methyl substituent (FIG. 5a-c), yielded nearly identical evolved sequences. Similarly, the sensors obtained for the closely related compounds 4F-MBMB and AB-PINACA contained identical mutations (data not shown).

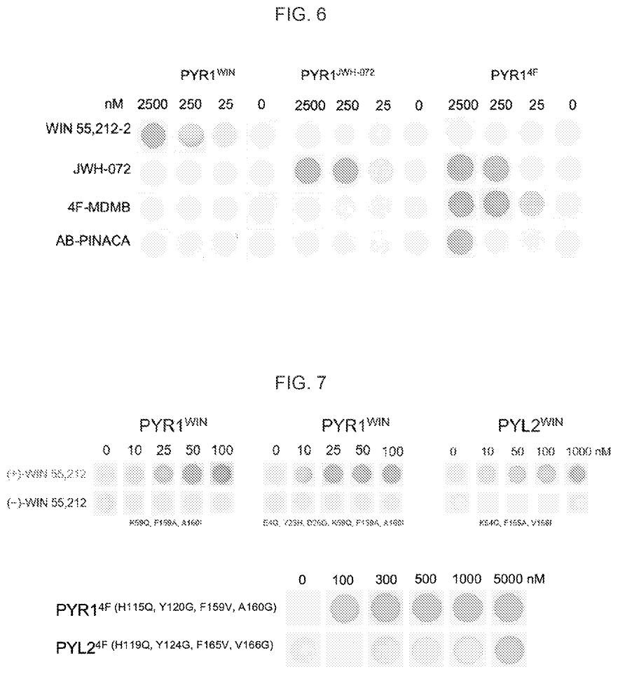

As most synthetic cannabinoids share a central indole or indazole scaffold, we anticipated that our evolved cannabinoid receptors might show cross-reactivity. To explore this, we tested three of our highest affinity sensors for cross-reactivity to cognate ligands (FIG. 6), and these tests indicate that PYR1^{WIN}, PYR1^{JWH-072}, PYR1^{4F} have good selectivity. PYR1 ^{WIN} recognizes WIN 55,212-2, the bulkiest ligand of the three sensors, and does not cross-react with either of the three off-target ligands tested. PYR1^{JWH-072} and PYR1^{4F} display modest cross-reactivity to low μM concentrations of both WIN 55,212-2 and AB-PINACA respectively. PYR1^{4F} cross-reacted most with JWH-072, however, in all cases on-target sensitivity was at least 10-fold higher than the off-target sensitivity. Thus, PYR1-derived sensors can provide both sensitive and selective ligand detection, although this will vary by receptor and chemical.

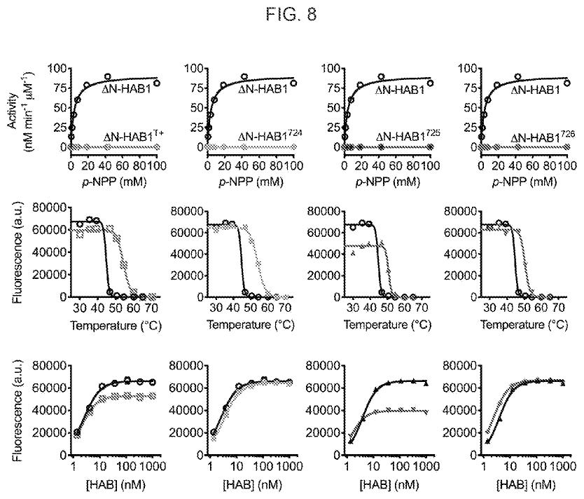

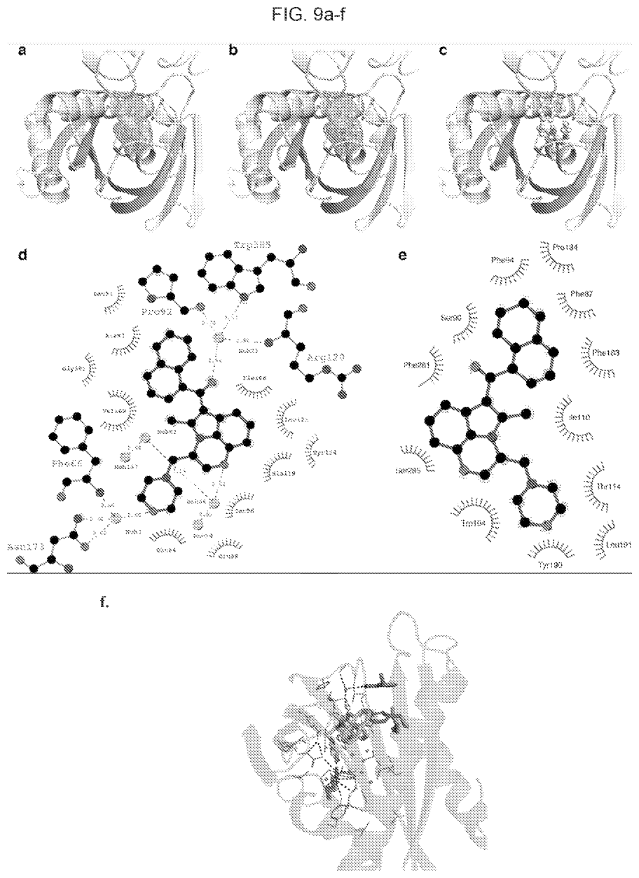

To understand the underlying molecular basis for cannabinoid recognition by our evolved receptors, we sought to obtain the structure of a receptor-cannabinoid-HAB1 complex and targeted two high-affinity sensors, PYR1^{4F} and PYR1^{WIN}. In our experience, PYL2 (a close relative of PYR1) forms crystals more readily than PYR1. We, therefore transposed mutations conferring ligand-selective responsiveness to PYL2, creating PYL2^{4F} and PYL2^{WIN}, which both retain nM ligand responsiveness (FIG. 7). In addition, we created a stabilized, catalytically inactive derivative of HAB1 less prone to oxidative inactivation by employing computational redesign, yielding ΔN-HAB1^{T+} (derived from a HAB1 truncation that contains its PYR1-binding catalytic domain). This variant harbors 15 mutations, displays a ~7° C. increase in apparent Tₘ, and retains high-affinity ligand-dependent binding to PYR1, as measured by a yeast surface display assay that employs PYR1^{M} (H60P, N90S) a monomeric double mutant optimized for yeast surface display ¹⁷ (FIG. 8, Table 4). Using these engineered proteins in matrix screens we obtained diffraction quality crystals for a ternary PYL2^{WIN}/WIN 55,212-2/ΔN-HAB1^{T+} complex, whose structure was solved by molecular replacement (1.9 Å resolution, Table 5). Diffraction quality PYL2^{4F} crystals were not obtained. Several rounds of structural refinement were carried out before modeling WIN 55,212-2 into the ligand-binding pocket's unbiased electron density (FIG. 9a-f). A real-space correlation coefficient of 0.967 calculated between the unbiased electron density and (+)-WIN 55,212-2 indicates agreement between the model and observed electron density. We note that the evolved receptor recognizes the biologically active (+)-WIN 55,212-2 stereoisomer, although selections were conducted using a racemate (FIG. 7).

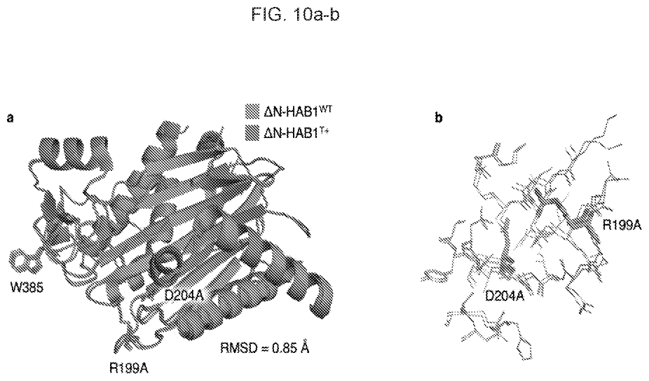

A central feature of ABA recognition by native sensors is the formation of a closed-receptor conformer that is stabilized by a hydrogen-bond network between a conserved water, ABA's ring ketone, main chain amides in the gate and latch loops, and the HAB1's W385 “lock” residue ¹⁸⁻²⁰. In our PYL2^{WIN} structure, WIN 55,212-2's naphthoylindole ketone functions analogously to ABA's ketone and is coordinated through water-mediated hydrogen bonds to backbone P92 in the gate, R120 in the latch, and HAB1's W385 lock residue (FIG. 2c). Binding is further stabilized by an extensive network of hydrophobic contacts and a water-mediated contact to WIN's morpholine oxygen (FIG. 9a-f). In comparison to PYR1, PYR1 ^{WIN} harbors 3 mutations (K59Q, F159A, A160I) and our structure illuminates their roles in allowing favorable binding. The most conspicuous effect is a relief of steric clash that would occur between F159 and WIN's naphthalene ring in a wild type receptor (FIG. 2d). The neighboring A160I mutation is positioned to enhance receptor-ligand surface complementarity by enabling the naphthoylindole to better pack in this position relative to the wild type receptor. The K59Q mutation appears to reduce the electrostatic penalty of burying WIN's positively charged morpholine ring, but also organizes a water-mediated hydrogen bond network at the base of the pocket (FIG. 2d and FIG. 9a-f). Thus, WIN's binding to PYL2^{WIN} involves a combination of polar and hydrophobic contacts, which contrasts with its binding mode in the human cannabinoid receptor CB2, where binding involves exclusively hydrophobic contacts and a more extended ligand conformer²¹ (FIG. 2e). In addition, the structure illustrates the success of our HAB1 redesign, showing that ΔN-HAB1^{T+}'s main chain is nearly superimposable with that of wild type (RMSD 0.85 Å) and that the key rotamers for residues involved in receptor interactions are maintained (FIG. 10a-b). Collectively, these data provide a mechanistic basis for the sensitive and selective cannabinoid recognition by our evolved receptor and illuminate the mutability of PYR1's ligand-binding pocket.

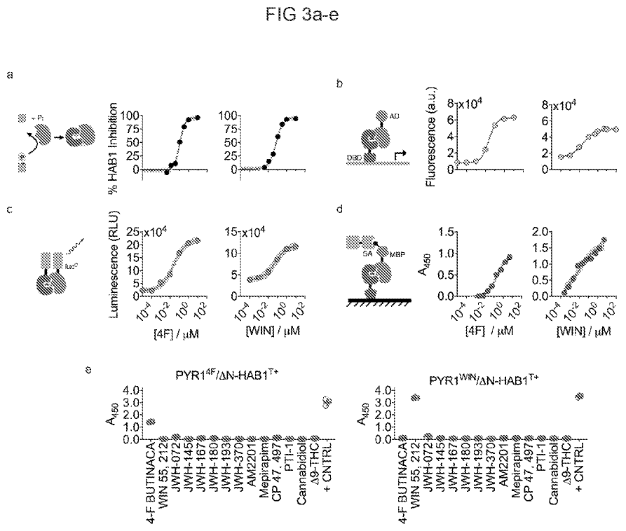

In principle, the PYR1-HAB1 CID mechanism enables rapid construction of multiple sense-response outputs, as has been demonstrated with other designed CID sensors 1 To explore the portability of the designed PYR1-HAB1 system, we selected two high-affinity receptors for evaluation of in vitro HAB1 inhibition, yeast transcriptional activation circuits, and in vivo protein fragment complementation with split luciferase. PYR1^{WIN} and PYR1^{4F} each exhibited nanomolar EC₅₀ values for HAB1 inhibition (PYR1^{WIN}, EC₅₀=72 nM [63, 81]; PYR14F, EC₅₀=102 nM [92, 122]; 95% confidence interval is shown in brackets; FIG. 3a). Fusion of the transcriptional activator VP64 to ΔN-HAB1 and a zinc finger DNA binding domain to PYR1^{4F} enabled inducible GFP expression from a synthetic cassette integrated into the genome of S. cerevisiae with an EC₅₀ of 23 nM [21, 24] (FIG. 3b). The same transcriptional circuit built with PYR1^{WIN} responded to WIN-55,212-2 with an EC₅₀ of 28 nM [20, 38]. Similar success was achieved with the NanoLuc split luciferase systems 22; NLuc^{1g}-PYR1^{4F}/NLuc^{sm}-ΔN-HAB1 responded with an EC₅₀ of 25 nM [15, 43], and NLuc^{N}. PYR1^{WIN}/NLuc^{C}-ΔN-HAB1 responded with an EC₅₀ of 56 nM [22, 187] (FIG. 3c), Taken together, these results show that the PYR1/ΔN-HAB1 CID mechanism enables portability to both in vitro and in vivo formats.

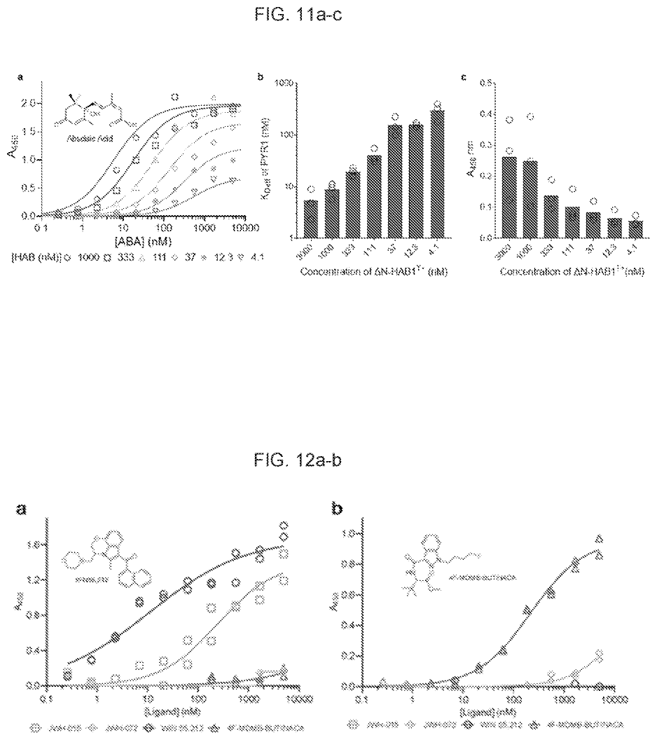

Synthetic cannabinoids are frequently modified to evade detection by routine drug testing. For example, 4F-MDMB-BUTINICA is a relatively new indazole that first appeared in 2018 and rapidly became one of the most prevalent synthetic cannabinoids used in the US ²³⁻²⁵. Although mass spectrometry methods can sensitively detect this and most synthetic cannabinoids, lower-cost and easier-to-use immunoassays (e.g., ELISAs) dominate routine drug testing. Given this, we sought to convert our CID system into an ELISA-like system for microplate format measurements. To do so, we developed a hybrid sandwich-assay in which the PYR1 sensor is surface-immobilized and then co-incubated with biotinylated ΔN-HAB1^{T+} and the ligand of interest. Detection of ternary complexes can then be quantified colorimetrically by enzyme activity using streptavidin-linked HRP, as is commonly done with ELISAs. We first developed and optimized an ABA-detection system using PYR1^{M}, which binds ABA, and tested it using differing amounts of both biotinylated ΔN-HAB1^{T+} and ABA concentrations. The optimized system produced lower limits of detection (LODs) for ABA as low as 2 nM (0.7 ng/mL) and showed an effective K_{d} for ABA of 9.7 nM (FIG. 11a-c, Table 5). Adopting this optimized format for PYR1^{4F-M} and PYR1^{WIN-M}, we were able to detect 4F-MDMB and WIN 55,212-2 with average LODs from two biological replicates of 6 nM (2.2 ng/ml) and 490 μM (0.21 ng/ml), respectively (FIG. 3d). In addition, minimal cross-reactivity between these sensors and a panel of 14 test cannabinoids was observed using this detection format (FIG. 3e, FIG. 12a-b). Combined with our other results, these data further demonstrate that PYR1's native CID mechanism can be harnessed to develop multiple sense-response outputs and demonstrate that our optimized ELISA-like test enables selective and sensitive detection of target ligands using evolved PYR1-based sensors.

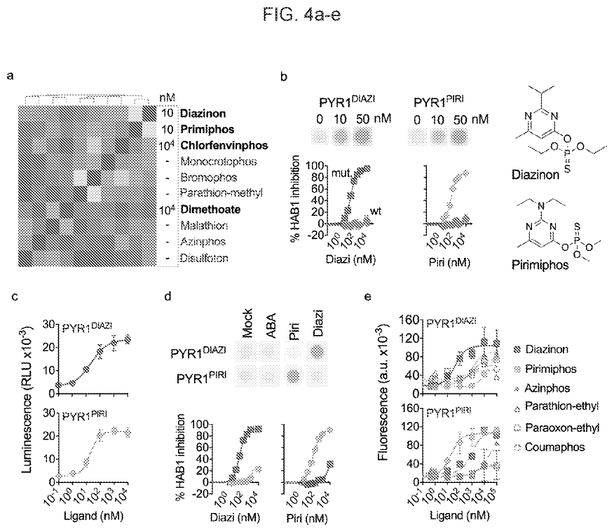

Given the success of the PYR1 scaffold as a platform for cannabinoid sensing, we sought to explore the possibility of rapidly generating new sensors for a second class of compounds. To do so, we screened single and multi-site mutational libraries (Table 6) against various different organophosphates, an important class of toxic, non-selective acetylcholinesterase inhibitors that were among the first insecticides broadly used in the 20th century. Due to their effects on non-target organisms, most organophosphates have been banned in the US, and they present an ongoing environmental monitoring challenge. We screened PYR1 libraries against a panel of 10 organophosphates (diazinon, pirimiphos-methyl, dimethoate, chlorfenvinphos, parathion, disulfoton, azinphos-ethyl, bromophos-methyl, malathion, and monocrotophos) and yielded receptors for diazinon, pirimiphos-methyl (pirimiphos), dimethoate, and chlorfenvinphos at concentrations between 5 and 100 μM (data no shown). To improve receptor affinity, we employed recombination-based mutagenesis and combined the primary hit receptors for diazinon, pirimiphos, chlorfenvinphos, and dimethoate and shuffled these against parent libraries. This approach was repeated four times, reducing the ligand concentration at each step to ultimately yield sensors that respond to 10 nM diazinon or pirimiphos (FIG. 4a,b). Receptors with 10 μM affinity, but not lower, were obtained for chlorfenvinphos or dimethoate. The diazinon-responsive variant PYR1^{DIAZI} is a heptuple mutant (E8G, V81Y, L87M, F108Y, M158V, F159G, A160V) and the pirimiphos-responsive variant PYR1^{PIRI} is an octuple mutant (K59R, S92M, N119S, S122Q, E130G, F159T, A160T, V174A). These optimized sensors were also immediately portable to the split luciferase system with low nM sensitivity (FIG. 4c). Together, these data demonstrate that the PYR1 ligand-binding pocket can mutate to accommodate organophosphate ligands and may provide a general system for developing organophosphate sensors.

To address the selectivity profiles of the evolved organophosphate receptors we first characterized their cross-reactivity to target ligands, given the close structural similarity of diazinon and pirimiphos. Both HAB1 inhibition and Y2H assays showed that the evolved receptors are highly selective to their on-target ligands (FIG. 4d). PYR1^{PIRI} was activated by low nM concentrations of pirimiphos but required high nM to low μM diazinon concentrations for activation above background levels. Similar results were observed with the PYR1^{DIAZI} receptor, and neither receptor was activated by ABA. In a strict test of specificity, we used our yeast-transcription circuit to characterize the off-target responses of these engineered receptors to a panel of six chemically similar organophosphates. The PYR1^{DIAZI} EC₅₀ to diazinon was greater than 10-fold lower than all other molecules profiled (FIG. 4e). For example, PYR1^{DIAZI} responded with an EC₅₀ of 1.1 AM to azinphos, but with an EC₅₀ of 43 nM to diazinon. Other off-target ligands responded with higher EC₅₀ values and with lower activation levels (Table 7). High selectivity was also observed with PYR1^{PIRI}, the off-target response from diazinon was the strongest with an EC₅₀ of 1.0 μM, 50-fold higher than that with pirimiphos.

Our data demonstrate that multi-site mutagenesis of PYR1 can be used to create libraries of receptors from which high-affinity sensors can be evolved. Using this system, we isolated 12 distinct receptor variants that yielded sensors for half of the screened cannabinoids and four distinct receptors for the 10 screened organophosphates; an overall hit rate of 47%. For the most part, our evolved sensors displayed good selectivity. For example, our PYR1^{4F} is more responsive to its evolved target ligand 4F-MDMB than the closely related indazole AB-PINACA. Evolved receptor selectivity profiles will undoubtedly vary case-by-case, nonetheless, our data illustrate that both high selectivity and sensitivity are achievable using PYR1 as a recognition scaffold. Combined with our prior work showing recognition of agrochemical ligands 8, our data suggest that PYR1's binding pocket is highly malleable, although the limits of its chemically accessible recognition space will need to be defined through further experimentation. We anticipate that hit rates can be improved by constructing higher-order mutant libraries. However, even without further improvement, the receptor evolution platform should accelerate the development of new sensors that can be used as modular building blocks for chemical-regulated controlling units in diverse biotechnology applications.

### Materials and Methods

### PYR1 Library Design

The design of the PYR1 pocket double mutant library was performed with a combination of mutant stability analysis with the Rosetta molecular modeling suite and manual curation. The final library design includes mutations at nineteen positions. Fifteen positions (59, 83, 89, 92, 94, 108, 117, 120, 122, 141, 159, 160, 163, 164, 167) were allowed to mutate to all amino acids except cysteine or proline. Four positions (62, 81, 87, 110) were restricted to smaller amino acid subsets.

### Construction of the PYR1 Double Site Mutant (DSM) Library

The PYR1 DSM libraries were constructed by nicking mutagenesis (NM) as previously described¹². Briefly, a library encoding pairs of mutations separated by at least eight amino acids was constructed by two sequential rounds of single-site NM using pooled primers (IDT). In parallel, an oligo pool (Agilent) containing primers encoding all pairs of proximate mutations (those separated by fewer than eight amino acids) was used in a single round of NM. The two libraries were then transferred to a two-hybrid vector by ligation and pooled to give the complete DSM library.

### Yeast-Two-Hybrid (Y2H) Screening of Mutagenized PYR1 Libraries

Selection experiments for mutant receptors that respond to new ligands were conducted as previously described ^{26,27}. Briefly, the PYR1 DSM mutant library was transformed into MAV99 harboring pACT-HAB1. Negative selections were conducted to remove receptors that bind HAB1 in a ligand-independent fashion (i.e., constitutive receptors) by growing the library on solid media containing 0.1% 5-fluoroorotic acid (5-FOA); the purged library was collected and used in subsequent selections for cells responsive to 30 μM of cannabinoid ligands (purchased from Cayman Chemical; Table 3) on SD-Trp,-Leu,-Ura media. Colonies supporting uracil-independent growth at 30° C. were isolated after 3 days, re-tested to confirm ligand-dependent growth on SD-Trp,-Leu,-Ura plates with and without test chemical, and then validated by β-galactosidase staining. Receptor ligand affinity optimization and new cannabinoid binders were obtained by generating and screening secondary shuffle (CB-S) libraries. To construct CB-S libraries, gene sequences of PYR1 cannabinoid-responsive variants were combined with the original PYR1 DSM library and the PYR1 SSM library in a ratio of 40/40/20, respectively, followed by recombinant-based mutagenesis, using nucleotide excision and exchange technology (NexT; see ref ²⁸). Resulting shuffled fragments were cloned into the Y2H pBD plasmid by restriction/ligation procedures. Ligation products were transformed into E. coli, colonies collected, and plasmid DNA extracted. In total, 3 independent shuffle libraries (CB-S1, CB-S2 and CB-S3) were generated at different stages of the optimization and rescreening process. CB-S1 and -S3 were screened with 10 to 0.1 μM ligand, depending on the sensitivity of the parental mutants. CB-S2 was screened with 0.05 to 0.025 μM ligand. Details for organophosphate sensor screening are provided in the supporting online materials.

### Ligand/Receptor-Mediated PP2C Inhibition Assays

Coding sequences of the cannabinoid sensors were cloned into the protein expression vector pET28a to encode 6×His-tag-receptor fusions. Constructs were sequenced and transformed into the BL21 (DE3) pLysS E. coli strain for heterologous expression with IPTG (1 mM), followed by purification, using affinity chromatography, as previously reported 29. In vitro validation of evolved sensors was performed by using recombinant sensors and ΔN-HAB1 (HAB1 A1-178, ref. 30) proteins, as previously described 27 Ligand/receptor-dependent inhibition of PP2C activity was performed essentially as previously reported using 25 nM ΔN-HAB1 with either 25 nM of titrated PYR1^{4F} or 50 nM of titrated PYR1^{WIN}. Cannabinoids concentration curves ranged from 4 to 10,000 nM and IC_{50S} for PP2C inhibition obtained via fluorescent measurement in the presence of 1 mM 4-methylumbelliferyl phosphate 31

### Cross Reaction Tests for High-Affinity Cannabinoid and Organophosphate Sensors

High-affinity sensors were tested for cross reaction using both Y2H and in vitro PP2C assays. PYR1^{WIN} and PYR1^{4F} sensors specificity was examined by X-gal staining using increasing amounts of WIN-55,212-2, 4-F-MDMB-BUTINACA, AB-PINACA and JWH-072 (0, 50, 100, 200, 500 and 1000 nM) and by in vitro PP2C inhibition assays (4-10000 nM) with EC₅₀ estimation for each receptor/ligand combination using nonlinear fitting with the [Agonist] vs. response function in GraphPad Prism 9. Similar methods were used to characterize PYR1^{PIRI} and PYR1^{WIN} sensor selectivity and sensitivity.

### Yeast Transcriptional Activation Circuits

PYR1 variants were used to drive gene expression in an inducible genetic circuit by fusing a zinc finger DNA binding domain (Z4³²) to the N-terminus of PYR1, and the VP64 activation domain 33 to the N-terminus of ΔN-HAB1. The SV40 nuclear localization signal was also fused to the N-terminus of PYR1. A single 2u plasmid was used to express SV40-Z4_{DBD}-PYR1 and VP64-HAB1, while the GFP expression cassette (Z44-CYC_{core}-GFP-CYC 1term) was integrated at the YPRCΔ15 site on chromosome XVI. GFP fluorescence induced by each circuit was measured 12 h after ligand addition to 1 mL cultures, 30° C. Fluorescence was measured by flow cytometry. Briefly, 50 μL of resuspended cells were transferred to a 96-well plate for analysis. The fluorescence intensity of each cell was measured using a BD Accuri C6 flow cytometer equipped with auto-loading. The forward scatter, side scatter, and GFP fluorescence (Ex/Em 488/533 nm) were recorded for a minimum of 10,000 events.

### In Vivo Split Luciferase Assays

To demonstrate a protein complementation output of PYR1-based cannabinoid sensors, the large and small fragments of split NanoLuc luciferase ²² were fused to the N-termini of PYR1 variants and HAB1, and expressed from a 2μ plasmid. Ligand was added to yeast cultures 12 h prior to measuring cell luminescence. A sample of each culture was diluted to an OD₆₀₀ of 0.2, 10 μL of which was transferred to a 96-well plate, the luciferase reagent mixture (Nano-Glo live cell substrate; Promega) diluted to a 1× concentration with DI water was added to the cell sample to a final volume of 200 μL. The relative luminescence signal (RLU) was measured using a Synergy™ Neo2 Multi-Mode Microplate Reader in luminescence detection mode for 30 minutes after the addition of the reagents. The RLU of each measured sample was taken as the average value of the time course once the signal had reached a plateau.

Construction of PYL2^{4F} and PYL2^{WIN}

The PYL2 coding sequence cloned in the Y2H pBD vector was used as template to incorporate the corresponding PYR1^{WIN} and PYR1^{4F} homologous residues via site directed mutagenesis using the QuikChange Lightning Multi Site-Directed mutagenesis kit (Agilent Technologies). Resulting clones PYL2^{WIN} (K64Q, F165A, V1661) and PYL2^{4F} (H119Q, Y124G, F165V, V166G) were sequence confirmed and incorporated into MAV99 harboring pACT-HAB1 and tested for ligand activation using uracil-independent growth and X-gal staining experiments. The same method was used to generate a PYL2^{WIN} version in a protein expression vector for crystallization studies.

### Expression, Purification, and Characterization of Variant HABs and PYRs

Except where noted, all proteins were expressed as N-terminal 6×His-MBP fusions primarily using the medium and method as described in ref. 17. For crystallographic structures, PYL2 and ΔN-HAB1^{T+} were expressed in E. coli and purified as described previously 27. Assays for phosphatase activity and apparent Tₘ, measured via thermal challenge using yeast surface display were performed essentially as previously described 17.

### HAB1 Stabilization

ΔN-HAB1^{T+} and other stabilized dead-ΔN HAB1 variants were designed as follows: loss of activity was encoded by either R199A, D243A or R199A and D204A; C186S. C274S mutations are known to improved redox stability ^{34,35}. Mutations conferring improved stability were identified by the Rosetta-based web server PROSS (all options default) using three separate HAB1 structures as starting points (PDB IDs: 4WV0, 4DS8, 3KB3) ³⁶ Consensus mutations shared between all structures were identified, yielding 22 potential mutations at 16 positions. These mutations were screened using empirical filters on distance from HAB1 interface and contact number 37. Manual curation was used to identify the final design set of 11 mutations at 11 positions.

### ELISA-Like Assays

PYR1 was immobilized overnight in clear plates, blocked with KPL milk diluent in buffer CBS ¹⁷ (CBSM). All binding reactions were performed using CBSM. Detection was performed via binding a streptavidin-HRP conjugate to biotinylated ΔN-HAB1^{T+} followed by incubation with a commercial TMB reagent. Lower limits of detection (LODs) were determined by linear regression of the response data in the low linear range using the formula LOD=3σ_{y}/m, where m is the slope of the response line and σ_{y} is the standard error of the y intercept using data combined from biological replicates.

### Crystallization

Purified protein was stored at −80° C. in a buffer containing 20 mM HEPES (pH 7.6), 50 mM sodium chloride, 10 mM DTT and 10% glycerol. Purified PYL2 and ΔN-HAB1^{T+} were mixed in 1:1.05 molar reaction and exchange into a buffer containing 20 mM HEPES (pH 7.6), 50 mM sodium chloride, 10 mM dithiothreitol, 5 mM magnesium chloride and 5% glycerol. The proteins were then concentrated to 15 mg/mL and incubated with a 5-fold molar excess of (+)-WIN 55,212 (Cayman Chemical, USA) for 30 minutes on ice. Crystallization of the PYL2: WIN: ΔN-HAB1^{T+} complex was conducted by sitting drop vapor diffusion at 19° C. Drops were formed by mixing equal volumes of the purified PYL2: WIN: ΔN-HAB1^{T+} complex with well solution containing 100 mM bis-tris propane pH 6.5, 200 mM sodium bromide and 19% (w/v) PEG 3,350. The resulting crystals were flash frozen after passing through a cryoprotection solution consisting of well solution plus 20% glycerol. X-ray diffraction data for each complex were gathered from a single crystal. Diffraction data was collected at 100 K using the LS-CAT ID-21-F beam line at the Advanced Photon Source (Argonne National Labs, Lemont, IL). Diffraction data were indexed, integrated, and scaled using the XDS software package 38

### Structure Determination

The PYL2: WIN: ΔN-HAB1^{T+} complex structure was solved by molecular replacement using a PYL2: Quinabactin: HAB1 complex (PDB ID 4LA7) devoid of ligand and water molecules as the search model to evaluate the initial phases. Phenix. AutoMR solved the initial phases and automatically built the majority of residues for both complexes ³⁹. The resulting models were completed through iterative rounds of manual model building in Coot (1) and refinement with Phenix.refine³⁹ using translational libration screw-motion (TLS) and individual atomic displacement parameters. A Phenix topology file for the (+)-WIN-55,512 ligand was generated using the PRODRG server (http://davapc1.bioch.dundee.ac.uk/cgi-bin/prodrg/)⁴⁰. Geometry of the final structures were validated using Molprobity. Data collection and refinement statistics for the final PYL2: WIN-55,212-2: ΔN-HAB1^{T+} model are listed in Table 5 and the coordinates for the structure deposited in the Protein Data Bank, PDB ID 7MWN.

### Additional Methodology/Materials

### Plasmids

Plasmids were constructed using either NEBuilder HiFi DNA Assembly Master Mix (New England Biolabs), using a Q5 Site-Directed Mutagenesis Kit (New England Biolabs), or by nicking mutagenesis. All kits were used according to the manufacturer's instructions. Nicking mutagenesis was performed as previously described (Wrenbeck et al. 2016). All PCR products used in NEBuilder assemblies were fractionated by agarose gel electrophoresis and purified using a Monarch DNA Gel Extract Kit (New England Biolabs) or Zymo Research Gel Extract Kit. For Q5 Site-Directed Mutagenesis, PCR products were similarly gel extracted and 20 ng was used in a 5 μl KLD reaction. Plasmids were transformed into Mach1 chemically competent E. coli (Invitrogen). All primers used are listed in Table 8, all plasmids are listed in Table 9, and all gblocks are listed in Table 10.

Plasmid pJS636 was constructed by NEBuilder HiFi assembly of pJS600 amplified with PJS-P2062/PJS-P2079, pJS600 amplified with PJS-P2080/PJS-P2081, and pJS600 amplified with PJS-P2082/PJS-P2063.

Plasmid pJS636-MBP was constructed by NEBuilder HiFi assembly of pJS636 amplified with pET29B+backbone_fwd/pET29B+backbone rev, pMAL-C5G_ccDockerin amplified with met-asn-His-MBP-TEV_fwd/met-asn-His-MBP-TEV_rev, and pJS636 amplified with dN-HAB1 fwd/dN-HAB1 rev.

Plasmid pJS645 was constructed by NEBuilder HiFi assembly of pYTK084 amplified with PJS-P2112/PJS-P2113 and pBD-PYR1-BbvCI amplified with PJS-P2114/PJS-P2115.

Plasmid pJS646 was constructed by NEBuilder HiFi assembly of pBD-PYR1-BbvCI amplified with PJS-P2116/PJS-P2117, pBD-PYR1-BbvCI amplified with PJS-P2118/PJS-P2119, pYTK047 amplified with PJS-P2120/PJS-P2121, and pBD-PYR1-BbvCI amplified with PJS-P2122/PJS-P2123.

Plasmid pJS647 was constructed by Q5 Site-Directed Mutagenesis of pJS645 using primers PJS-P2124/PJS-P2125.

Plasmid pJS678 was constructed by single/multi-site nicking mutagenesis of pJS636-MBP with primer HABstarR199AD204A.

Plasmid pJS723 was constructed by NEBuilder HiFi assembly of pJS678 amplified with PJS-P2244/PJS-P2243, gBlock PJS-G0023, and gBlock PJS-G0024.

Plasmid pJS724 was constructed by NEBuilder HiFi assembly of pJS678 amplified with PJS-P2244/PJS-P2243, gBlock PJS-G0023 amplified by PJS-P2246/PJS-P2247, gBlock PJS-G0023 amplified by PJS-P2248/PJS-P2249, and gBlock PJS-G0024.

Plasmid pJS725 was constructed by NEBuilder HiFi assembly of pJS678 amplified with PJS-P2245/PJS-P2243, gBlock PJS-G0025, and gBlock PJS-G0026.

Plasmid pJS726 was constructed by NEBuilder HiFi assembly of pJS678 amplified with PJS-P2245/PJS-P2243, gBlock PJS-G0025 amplified by PJS-P2246/PJS-P2250, gBlock PJS-G0025 amplified by PJS-P2251/PJS-P2249, and gBlock PJS-G0026.

Plasmid pJS753 was constructed by multi-site nicking mutagenesis of pJS637 with primers PJS-P2335 and PJS-P2336.

Plasmid pJSS754 was constructed by multi-site nicking mutagenesis of pJS637 with primers PJS-P2336, PYR1-Y120H-CAT, and PYR1-A160G-GGT.

Plasmid pJS755 was constructed by NEBuilder HiFi assembly of pJS723 amplified with primers PJS-P2337/PJS-P2338 and pJS637 amplified with PJS-P2339/PJS-P2340.

Plasmid pJS756 was constructed by NEBuilder HiFi assembly of pJS723 amplified with primers PJS-P2337/PJS-P2338 and pJS624 amplified with PJS-P2339/PJS-P2340.

Plasmid pJS794 was constructed by NEBuilder HiFi assembly of pJS755 (template for all) amplified with PJS-P2337/PJS-P2338, PJS-P2339/PJS-P2340, PJS-P2401/PJS-P2395, PJS-P2334/PJS-P2402, and PJS-P2335/PJS-P2340.

Plasmid pJS796 was constructed by NEBuilder HiFi assembly of pJS755 (template for all) amplified with PJS-P2337/PJS-P2338, PJS-P2339/PJS-P2403, PJS-P2404/PJS-P2405, and PJS-P2406/PJS-P2340.

Plasmid pSW004 was constructed by NEBuilder HiFi assembly in four steps. First, TEF1 promoter and TDH1 terminator amplified from the S. cerevisiae genome (primers SW019/SW020 and SW023/SW024), and PYR amplified with SW021/SW022 were assembled with the pIW15 plasmid backbone digested with Pfol. Second, HAB1 amplified with SW017/SW018 was assembled with the first plasmid digested with Not1 and Spel. Third, SV40-ZF4 (gblock SW-G001) was assembled with the plasmid digested with Not1. Finally, VP64 (gblock SW-G002) was assembled with the plasmid digested with AvrII. PYR1 variants (PYR1^{4F} and PYR1^{WIN}) were amplified using SW021/SW022 and assembled with pSW004 digested with NheI and SacII. Plasmids expressing PYR1 organophosphate variants (PYR1^{diazi} and PYR1^{Piri}) were amplified using SW007/SW008 and assembled in a similar manner as PYR1^{4F} and PYR1^{WIN}

The GFP expression cassette was integrated into the YPRCΔ15 site on chromosome XVI of S. cerevisiae with plasmid pSW010, which was constructed from pIW156 that contains a HIS3 selection marker. NEBuilder HiFi assembly of fragments encoding 700 bp of upstream homology was amplified with SW025/SW026 from the S. cerevisiae genome, while the downstream homology region was amplified with SW035/SW036. Other fragments include, the Z4 DNA binding domain sequence fragment (gblock SW-G003), CYC1 core promoter amplified with SW027/SW028, and GFP amplified with SW029/SW030.

Split NanoLuc luciferase plasmid pSW143 was constructed from pSW004 in two steps with NEBuilder HiFi assembly. First, the SmBit-linker gblock (SW-G004) and pSW004 digested with Avril and NheI were assembled. Second, LgBit-linker glock (SW-G005) and TEF1 promoter amplified with SW646/SW647 were assembled with the first step plasmid digested with EcorI and SacI. These manipulations create a system where the small NanoLuc protein fragment is fused to the N-terminus of HAB1, while the large protein fragment is fused to the N-terminus of PYR1. Plasmid expressing PYR1 variants (PYR1^{4F} and PYR1 ^{WIN}) were amplified using SW422/SW423 and assembled with pSW143 digested with AvrII and NheI. Plasmids expressing organophoshate PYR1 variants (PYR1^{DIAZI} and PYR1^{PIRI}) were amplified using SW043/SW044 and assembled in a similar manner as PYR1^{4F} and PYR1^{WIN}

### PYR1 Double Mutant Library Design

### Selection of Residues to Mutate

We examined PYR1 bound to ABA (PDB ID 3K90, chain A) and selected all residues either:
with any atom within 5 Å of ABA, or
with any atom within 6 Å of ABA and a Cα-Cβ vector not directed away from ABA.

After manual curation of the resulting list of positions, we removed the following residues:
H60: Faces away from ABA and involved in PYR1 dimerization (Dupeux et al. 2011)
F61: Outward facing and involved in PYR1 dimerization (Elzinga et al. 2019).
R79: Appears to have only second-shell effects on ligand binding.
P88: Conserved in the gate loop (Melcher et al. 2009).
T91: Outward facing.
H115: Conserved in the latch loop (Melcher et al. 2009).

### Stability Prediction in Rosetta

We first relaxed the PYR1 structure into the Rosetta score function using the following command:

-/Source/Rosetta/main/source/bin/relax.macosclangrelease \
-corrections::beta_nov16 \
-ex1 \
-ex2 \
-relax:constrain_relax_to_start_coords \
-relax:coord_constrain_sidechains \
-relax:ramp_constraints_false \
-use_input_sc \
-flip_HNQ \
-no_optH_false \
-s_AtPYR1.pdb \
-out:no_nstruct_label \
-out:suffix _relaxed \
-out:file:o AtPYR1_relaxed.pdb \
-out:file:scorefile AtPYR1_relaxed_score.sc

The input file At PYR1.pdb is chain A from PDB ID 3K90.

Next, we ran FilterScan in Rosetta to estimate the ΔΔG of all possible point mutations at each designed position. We used the following RosettaScripts XML:

```
<ROSETTASCRIPTS>
  <SCOREFXNS>
    <ScoreFunction name=“f_soft” weights=“beta_nov16_soft”/>
    <ScoreFunction name=“f_hard” weights=“beta_nov16”/>
  </SCOREFXNS>
  <RESIDUE_SELECTORS>
    <Index name=“residues”
        resnums=“%%pocket_residues%%”/>
  </RESIDUE_SELECTORS>
  <TASKOPERATIONS>
    <InitializeFromCommandline name=“init”/>
    <OperateOnResidueSubset name=“residue_subset”
                  selector=“residues”>
      <AddBehaviorRLT behavior=“dummy”/>
    </OperateOnResidueSubset>
    <OperateOnResidueSubset name=“residues_to_ignore”>
      <Not selector=“residues”/>
      <RestrictToRepackingRLT/>
    </OperateOnResidueSubset>
  </TASKOPERATIONS>
  <MOVE_MAP_FACTORIES>
  </MOVE_MAP_FACTORIES>
  <SIMPLE_METRICS>
    <TotalEnergyMetric name=“energy_metric”
              scorefxn=“f_hand”/>
  </SIMPLE_METRICS>
  <MOVERS>
    <MinMover name=“min_mover”
          scorefxn=“f_hard”
          chi=“true”
          bb=“true”
          tolerance=“0.001”
          max_iter=“1000”/>
  </MOVERS>
  <FILTERS>
    <SimpleMetricFilter name=“energy_filter”
                metric=“energy_metric”
                comparison_type=“lt”
                cutoff=“9999”
                confidence=“0”/>
    <Delta name=“delta_energy”
        filter=“energy_filter”
        upper=“true”
        lower=“false”
        range=“−9999”/>
    <FilterScan name=“scan”
            scorefxn=“f_soft”
            task_operations=“init,residue_subset,residues_to_ignore”
            relax_mover=“min_mover”
            score_log_file=“%%score_log_file%%”
            delta_filters=“delta_energy”/>
  </FILTERS>
  <PROTOCOLS>
    <Add filter_name=“scan”/>
  </PROTOCOLS>
  <OUTPUT/>
</ROSETTASCRIPTS>
```

And the following options:

-corrections::beta_nov16
-ex1
-ex2
-out:nooutput
-parser:protocol scan.xml
-parser:script_vars pocket_residues=59,62,81,83,87,89,92,94,108,110,117,120,122,141,159,160,163,164,167
-s AtPYR1_relaxed.pdb

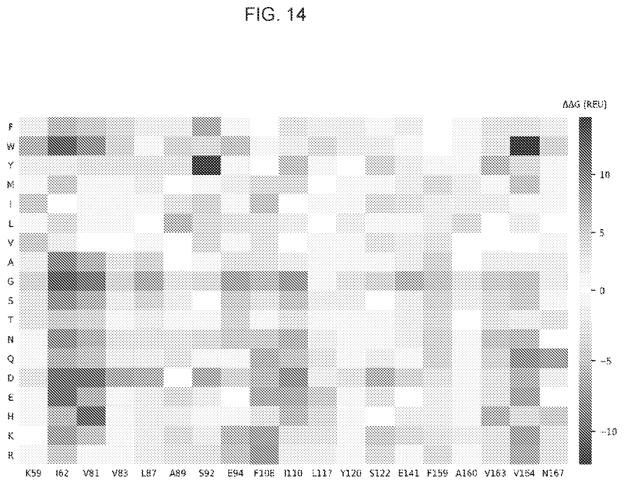

Results are shown in FIG. 14.

### Final Library Design

To decrease the size of the library, we pruned mutations at positions where mutations appear particularly destabilizing or that appeared, by inspection of the structure, less likely to yield hits. Specifically, we removed mutations:
At position 62 with ΔΔG>7 REU,
At position 110 with ΔΔG>5 REU,
At position 120 with ΔΔG>5 REU, and
At position 160 with ΔΔG>1 REU.

In addition, we restricted mutations at position 87 to hydrophobic amino acids and glycine. These restrictions yielded the final library design given in Table 1.

### Construction of PYR1 Mutant Libraries

The PYR1 double mutant library (DSM) was constructed by nicking mutagenesis (Wrenbeck et al. 2016). The template used was pJS647, which contains the mutagenized portion of PYR1 flanked by BsaI sites and a BbyCI site for nicking. The full double mutant library was constructed in two parts. First, to construct a library of all distant mutations (those separated by more than eight amino acids), individual oligos encoding each mutation flanked by 24 bp of homology to the WT PYR1 sequence were pooled and used to construct a library of PYR1 single mutants (L004). This library was used as input to a second round of mutagenesis with the same pool of oligos, yielding a library of distant double mutants (L006). Second, to construct a library of all proximate mutations (those separated by fewer than eight amino acids), an oligo pool containing primers encoding all such proximate double mutations was synthesized (Agilent). This pool was used directly to perform nicking mutagenesis as previously described (Wrenbeck et al. 2016), yielding a library of all proximate double mutants (L005). After construction of libraries in pJS647, the PYR1 cassettes were transferred to pJS646 to reconstitute the full PYR1 Y2H vector. Briefly, library DNA and pJS646 were digested with BsaI-HFv2 (NEB) and fractionated by agarose gel electrophoresis. Bands corresponding to the 0.4 kb PYR1 cassettes (L005 and L006) and 6.6 kb vector (pJS646) were excised from the gel and DNA was recovered using a Monarch DNA Gel Extraction Kit (NEB). The library cassettes (7.6 ng) were ligated into the Y2H vector (82 ng) for 1 hour at 22° C. in a 10 μL reaction using T4 DNA ligase (NEB). The ligation reaction was purified using a Monarch PCR & DNA Cleanup Kit (NEB) and transformed into XLI-Blue electrocompetent E. coli (Agilent). Finally, the two libraries were pooled to give the full double-mutant library.

Two separate libraries were screened to isolate organophosphate sensors, a double mutant combination (DMC) library and a newly constructed PYR1-K59R-NNK library. The single site saturation K59R library was constructed using the PYR1-K59R mutant receptor template (Park et al. 2015) as three sub-libraries targeting positions 1-64, 65-128, or 129-191 using nicking mutagenesis (Wrenbeck et al. 2016) with degenerate NNK oligos spanning the entire coding sequence except R59 and H60. The PYR1-K59R-NNK library coverage was confirmed at greater than 99.9% by deep sequencing of libraries (library statistics are reported in Table 7). The PYR1 DMC library that we screened was previously constructed using oligo pool mutagenesis and (Medina-Cucurella et al. 2019) comprises combinations of 185 unique single mutations at 17 PYR1 positions (Park et al. 2015). The library shows 100% coverage of all 185 single mutants and 77% coverage (8,316/10,845) of the potential double mutants.

### PYR1 Library Sequencing

Library coverage for the two final libraries (L007 and L008) was verified by deep sequencing. Briefly, 1 ng of library DNA was PCR amplified with primers PJS-P2165 and PJS-P2166 using 2xQ5 Master Mix (NEB) for twelve cycles with an annealing temperature of 64° C. and an extension time of 60 seconds. The product was purified with a Monarch PCR & DNA Cleanup Kit (NEB). Next, the libraries were indexed by PCR amplifying Ing of the purified product with Illumina primers RPI and RPI1 (for L007) or RPI2 (for L008) using the same conditions. These reactions were purified using a Monarch PCR & DNA Cleanup Kit and then fractionated by agarose gel electrophoresis. The desired bands (503 bp) were excised and the DNA purified using a Monarch Gel Extraction Kit (NEB). Finally, the amplicons were further purified using AmpureXP beads according to the manufacturer's instructions. Deep sequencing was performed by the University of Colorado Boulder Biofrontiers Sequencing Core using an Illumina MiSeq with a 250 cycle paired end kit. Sequences were analyzed using custom scripts.

### Yeast-Two-Hybrid Screen of DSM Library for Cannabinoid Sensors

Selection experiments for mutant receptors that respond to new ligands were conducted as previously described (Peterson et al. 2010; Park et al 2015). Briefly, the PYR1 DSM mutant library was transformed into MAV99 (pACT-HAB1) by electroporation on (Lin et al. 2011). Negative selections were conducted to remove receptors that bind HAB1 in a ligand-independent fashion (i.e., constitutive receptors) by growing the library on petri plates containing 0.1% 5-fluoroorotic acid (5-FOA); the purged library was collected and used in subsequent selections for cells responsive to 30 μM of the synthetic cannabinoids (JWH-015, JWH-007, JWH-016, JWH-018, JWH-030, JWH-072, JWH-133, JWH-145, JWH-167, JWH-180, JWH-193, JWH-210, JWH-370, ADBICA, AM2201 8-quinolinyl carboxamide, Mepirapim, AB-FUBINACA, AB-PINACA, (+)-CP 47,497, (+)-WIN 55,212, FUB-PB-22, PTI-1, Cannabidiol and A9-THC) by growth on SD-Trp,-Leu,-Ura media (SIGMA); ~2×10⁶ cells were plated per 100×15 mm petri dish. Colonies supporting uracil-independent growth at 30° C. were isolated after 3 days, re-tested to confirm ligand-dependent growth on SD-Trp,-Leu,-Ura plates with and with test chemical, and then validated presence/absence of test chemical by β-galactosidase staining using the chloroform-agarose method. Plasmids from validated hits were isolated, propagated in E. coli, and sequenced. This process yielded double mutant hits for pM concentrations of JWH-015, JWH-016, JWH-018, JWH-030, JWH-072, WIN55,212 and CP 47,497. Additional cannabinoids 4-fluoro MDMB-BUTINACA, Cannabigerolic Acid, Cannabidiolic Acid and THCA-A were screened with DSM shuffled libraries as described below.

### Construction of Secondary Shuffled (CB-S) Libraries

Plasmid DNAs of PYR1 cannabinoid-responsive variants were combined with the original PYR1 DSM library and the PYR1 SSM “pocket” library (Park et al 2015) in a ratio of 40/40/20 respectively, followed by recombinant-based mutagenesis, using nucleotide excision and exchange technology (NexT; (Müller et al. 2005)). Resulting shuffled fragments were cloned into the Y2H pBD plasmid by restriction/ligation procedures. Ligation products were transformed into E. coli, colonies collected, and plasmid DNAs extracted. In total, 3 independent optimization shuffle libraries (CB-SI, CB-S2 and CB-S3) were generated at different stages of the optimization and rescreening process as follow: CB-SI included DNA templates from PYR1 double mutants F159A/A160I, F159A/V164H, Y120G/A160G, Y120H/A160G and Y120A/A160G. CB-S2 included DNA templates from PYR1 mutants K59N/Y120H/A160G, V81I/Y120G/A160G, K59N/Y120H/A160G, Y120G/M158I/A160G, K59Q/F159A/A160I, K59Q/F159A/A160I/Q169R, Y23H/D26G/K59Q/F159A/A160I. CB-S3 included DNA templates from PYR1 K59S/V83W/Y120G/F159A, Y120G/F159L/A160G, Y120G/F159V/A160G, Y120G/F159L/A160G, E4G/K59R/H115Q/Y120G with the PYR1 SSM and DSM libraries.

### Screening of (CB-S) Libraries for Improved Affinity and More Cannabinoid Binders

All CB-S libraries (Except CB-S2) screenings for sensor/ligand affinity optimization were done in a range of concentrations (10-0.1 μM) depending on the sensitivity of the parental mutants tested at least otherwise stated. The CB-SI library was screened on JWH-015, JWH-016, JWH-018, JWH-030, JWH-072, WIN 55,212. This enabled the identification of seven independent clones responding to cannabinoid concentrations as low as 50 nM for JWH-016 (PYR1 V81I/Y120G/A160G), JWH 072 (PYR1 K59N/Y120H/A160G), WIN 55,212 (PYR1 K59Q/F159A/A160I, PYR1 K59Q/F159A/A160I/Q169R, PYR1 Y23H/D26G/K59Q/F159A/A160I); to 100 nM for JWH-015 (PYR1 K59N/Y120H/A160G), and to 500 nM for JWH-018 (PYR1 Y120G/M158I/A160G).

The CB-SI library was also screened on 30 μM of cannabinoids JWH-007, JWH-145, JWH-167, JWH-180, JWH-193, JWH-210, JWH-370, ABDICA, AM2201 8-quinolinyl carboxamide, Mepirapim, AB-FUBINACA, AB-PINACA, FUB-PB-22, PTI-1, Cannabidiol, A9-THC, Cannabigerolic Acid, Cannabidiolic Acid and THCA-A. This experiment yielded sensors responding to μM concentrations of cannabinoids JWH-007 (PYR1 K59S/V83W/Y120G/F159A), JWH-167 (PYR1 Y120G, F159L, A160G), JWH-193 (PYR1 Y120G/F159V/A160G), AB-PINACA (PYR1 Y120G/F159L/A160G) and the phytocannabinoid A9-THC (E4G/H115Q/Y120G/A160G).

The CB-S2 library was screened on low (50 nM, 25 nM) concentrations of cannabinoids JWH-015, JWH-016, JWH-018, JWH-072, WIN SS,212 and JWH-030 (250 nM). This experiment enabled the identification of sensors with improved low nM affinity for JWH-015 (PYR1 K59N/Y120H/F159L/A160G), JWH-016 (PYR1 V81I/Y120G/F159L/A160G), JWH-072 (PYR1 E4G/K59N/Y120H/F159L/A160G) and WIN 55,212 (PYR1 E4G/Y23H/D26G/K59Q/F159A/A160I).

The CB-S3 library, was screened on decreasing concentrations (10, 1, then 0.1 μM) of cannabinoids JWH-007, JWH-167, JWH-193, AB-PINACA, (+)-CP 47,497, 49-THC, Cannabidiol, Cannabigerolic Acid, Cannabidiolic Acid, THCA-A and 4-fluoro MDMB-BUTINACA. This screening resulted in clones with improved affinity to JWH-007 (PYR1 V83W/Y120G/F159A/A160I), JWH-167 (PYR1 Y58H/V81M/V83F/H115Q/Y120G/F159L/A160G/D184G), JWH-193 (PYR1 L87M/A89V/E102K/Y120G/F159V/A160G), AB-PINACA (PYR1 E4G/H115Q/Y120G/F159V/A160G), A9-THC (E4G/K59R/H115Q/120G) and to cannabinoids 4-fluoro MDMB-BUTINACA (PYR1 H115Q/Y120G/F159V/A160G) and CBDA (PYR1

### General Expression and Purification of Variant HAB1s and PYRs

Proteins described in this work were expressed as N-terminal 6×His-MBP fusions primarily using the medium and method as described in (Steiner et al., 2020), with following noted exceptions. Proteins used in the experiments described by FIG. 3a were produced as in (Vaidya et al. 2019).

For catalytically dead HAB1 designs and all variants of PYR1, the supplemental MnCl₂ was excluded from ZY medium. Cell pellets were resuspended in Lysis Buffer at 4 mL g⁻¹ wet cell weight and allowed to incubate for 15 minutes at bench temperature (20° C.). Lysis buffer was comprised of 50% v/v B-PERII (ThermoFisher), 10% w/v glycerol, 50 mM HEPES, 200 mM KCl, 0.5 mM DTT, 5 mM TCEP, 10 mM imidazole, 15 mM MgCl₂, 0.2 U benzonase mL⁻¹, 0.1 mg mL⁻¹ lysozyme, and 1 mM PMSF dissolved in DMSO to 100×, with the buffer adjusted to pH 8.0 with KOH as required. Additionally, 10 mM MnCl₂ was added to all buffers for preparation of catalytically active HAB1 variants, and for preparations of catalytically dead HAB1 variants when they were intended for direct comparison to catalytically active HAB1 designs. When MnCl₂ is included, a brown color may be observed. The clarified supernatant from 0.5 L of expression culture was incubated with gentle rocking for 1 hour with 5 mL of washed Ni-NTA resin (G-Biosciences) suspended in wash buffer supplemented with 5 mM ATP (JK Scientific Cat: 438666). The resin was allowed to settle by gravity on ice, and the supernatant was decanted into fritted columns. Once the resin was fully settled and the unbound fraction drained, S bed volumes of wash buffer were applied, comprised of 10% w/v glycerol, 50 mM HEPES, 500 mM KCl, 0.5 mM DTT, 5 mM TCEP, and 20 mM imidazole, at pH 8.0. Bound proteins were eluted with 2 column volumes of elution buffer, composed of 10% w/v glycerol, 50 mM HEPES, 200 mM KCl, 5 mM TCEP, and 500 mM imidazole, at pH 8.0.

On ice, ammonium sulfate powder was added to the elutions to 80% saturation as determined by the following tool (www.encorbio.com/protocols/AM-SO4.htm). These mixtures were incubated with gentle rocking for 20 minutes, followed by centrifugation for 10 minutes at 20,000 g. The supernatant was removed by pipet, with a few minutes allowed for additional drainage of supernatant from tube and pellet. The pellets of proteins to be biotinylated were redissolved into biotinylation buffer, which for all MBP tagged proteins was pH 7.5, comprised of 10% w/v glycerol, 50 mM HEPES, 200 mM KCl, 5 mM TCEP. Proteins were desalted and quantified as previously described (Steiner et al 2019). All proteins to be biotinylated were either diluted or concentrated to 100 μM as required, and all MBP tagged proteins were biotinylated at a ratio of 20:1 at room temperature for 30 minutes. Biotinylation reactions were quenched as previously described and precipitated with saturated ammonium sulfate in 50 mM HEPES, 200 mM KCl, pH 8.0 to 80% saturation, with addition of DTT and TCEP as needed to maintain 1 mM of each. Suspensions were incubated on ice for 20 minutes, and pelleted by centrifugation for 10 minutes at 20,000 g. Supernatants were removed and pellets were dissolved into desalting buffer comprised of 10% w/v glycerol, 50 mM HEPES, 200 mM KCl, 1 mM DTT, 5 mM TCEP at pH 8.0, desalted, and quantified again. Quantified proteins were precipitated with saturated ammonium sulfate with DTT and TCEP added to 1 mM each, incubated on ice, and pelleted by centrifugation at 10,000 g for 15 minutes. The pellets were resuspended using saturated ammonium sulfate to a protein concentration of 100 AM, DTT and TCEP were added to 1 mM each, and the suspensions were stored at 4° C.

Proteins not getting biotinylated were redissolved directly into desalting buffer and were also quantified. These were precipitated by addition of ammonium sulfate powder to 80% saturation or greater. The suspensions were incubated on ice for 20 minutes and pelleted once again by centrifugation, this time at 10,000 g for 15 minutes. The supernatants were removed, and the pellets resuspended using saturated ammonium sulfate to a protein concentration of 100 μM, DTT and TCEP were added to 1 mM each, and the suspensions were stored at 4° C.

### GFP Activation and Fluorescence Measurements

Transformed yeast cells were acquired by first inoculating 5 mL of liquid media and incubating in a shaker incubator overnight at 30° C. Cells from the overnight culture were then used to inoculate a 50 mL culture with an initial cell density of 5×10⁶ cells/mL. This culture was grown at 30° C. with 200 rpm shaking until ~2×10⁷ cells/mL were produced, approximately 3 to 5 hours. Cells were harvested by centrifugation at 4000 rpm for 5 min, washed with 25 mL sterile, deionized water, resuspended in 1 mL of 100 mM LiAc, and transferred to a 1.5 mL tube, producing a cell suspension with approximately 10⁹ cells/mL. The cell suspension was briefly mixed by vortexing and 100 μL was transferred into a clean 1.5 mL tube for each transformation. Cells were pelleted at 13000 rpm for 15 sec and the supernatant was removed. A transformation mixture of 240 μL PEG (50% w/v), 36 μL 1.0 M LiAc, 50 μL salmon sperm DNA (2.0 mg/ml), plasmid, and sterile water were added to the cell pellet and mixed by vortexing for 1 min (total volume of 360 μL). The mixed transformation solution was then heat shocked at 42° C. for 40 min. Post heat-shock, cells were recovered by centrifugation at 8000 rpm for 30 sec. Cells resuspended in 400 μL sterile water were plated on appropriate selection media, in this case agar-SD media without uracil. Plates were incubated at 30° C. until mature colonies were formed.

Three single transformants were picked from selection plates, inoculated in 2 mL of SD-Ura containing 2% glucose in 14 mL culture tubes. After 24 hours of growth at 30° C. in a shaker incubator (200 rpm), the OD₆₀₀ of each sample was measured and cultures were back diluted to OD_{600=0.2} in fresh SD-Ura, the total volume of diluted culture was 9 mL. Each 9 mL culture was separated into 8 unique cultures by transferring 1 mL into 8 different wells of a 96 deep-well plate. Each well was used to evaluate the effect of a specific concentration of ligand. After all the cultures were transferred, 1 μL of stock ligand solution (each stock varying in concentration) was added to each well. The plate was sealed with an air-breathable polymer film and cultured with 1000 rpm shaking at 30° C. for 12 hours. Shaker humidity was maintained at 90%. Cells were harvested by centrifuge at 5000 g for 10 min, and after discarding the supernatant, the cells were suspended in 1 mL PBS buffer and centrifuged at 5000 g for 10 min. The cells were washed with 1 mL PBS buffer twice and resuspended in 1 mL DI water for flow cytometry analysis. For flow cytometry analysis, 50 μL of resuspended cells were transferred to a 96-well plate with flat bottom, adding DI water up to final volume 200 μL. The fluorescence intensity of cells within each sample was measured using a BD Accuri C6 flow cytometer equipped with auto-loading from 96 well plates. The forward scatter, side scatter, and GFP fluorescence (Ex/Em 488/533 nm) were recorded for a minimum of 10,000 events.

### Luminescence Assays

A protein complementation output assay of evolved PYR-based sensors was carried out by NanoLuc split luciferase (19). The larger fragment, LgBit (or Luc^{N}), was fused to N-terminus of PYR1 variants, while the small fragment, SmBit (or Luc^{C}) was fused to N-terminus of ΔN-HAB1. Expression of both PYR1 variants and ΔN-HAB1 was accomplished from a single 2μ plasmid. gBlock encoding the luciferase fragments were purchased from Integrated DNA Technologies (IDT). This plasmid was then transformed into S. cerevisiae BY4742, cells were grown in 2 mL synthetic-defined medium without uracil (SD-U) containing 6.7 g L″1 yeast nitrogen base without amino acids (DB Difco®; Becton-Dickinson), and 1.92 g L″1 yeast synthetic drop-out medium supplements without uracil (Sigma-Aldrich). Ligand was added to yeast cultures 12 hour prior to measuring cell luminescence. Each culture was diluted to an OD₆₀₀ of 0.2, 10 μL of which was transferred to a 96-well plate to make a final volume of 180 μL. 20 μL of the luciferase reagent mixture (Nano-Glo live cell substrate; Promega) was added to each well and gently mixed. The relative luminescence signal (RLU) was immediately measured continuously in a Synergy™ Neo2 Multi-Mode Microplate Reader over 30 minutes. The average value of RLU of each measured sample was taken once the measurements had reached a stable value. Three single transformants were picked from selection plates as done for the fluorescent measurements.

### Modifications to the General Expression and Purification Protocol for PYR^{4F-E}

Prior to centrifugation of cell pellets, flasks of cells to be harvested were incubated on ice for 30 minutes. All further steps were performed on ice, using buffers, tubes, and columns pre-incubated on ice, and the lysis buffer was further supplemented to 2.0 U benzonase mL⁻¹, 1.0 mg mL⁻¹ lysozyme. Lysis was allowed to proceed for 2 hours, rather than 15 minutes.

### Determination of Apparent Tₘ for HAB1 Variants

Citrate-buffered saline was first prepared (CBS: 20 mM sodium citrate, 147 mM NaCl, 4.5 mM KCl). A portion of this CBS was removed and this aliquot was made into CBS⁺⁺⁺ by addition of freshly dissolved DTT to 1 mM, TCEP pH 8.0 to 1 mM, and MnCl₂ to 10 mM followed by pH adjustment to 8.0 using 1 M sodium hydroxide. CBS⁺⁺⁺ was then sterile filtered and placed on ice. A further portion of CBS was removed and the above reagents were added in addition to bovine serum albumin at 0.2% w/v to produce CBSFF⁺⁺⁺, followed by adjustment of pH to 8.0 with 1 M sodium hydroxide and the solution was again sterile filtered and placed on ice. The remainder (and bulk) of the CBS was used to produce CBSF by addition of 0.1% w/v BSA with adjustment of pH to 8.0 using 1 M sodium hydroxide, and this solution was also sterile filtered, and 25 mL were retained at room temperature, with the bulk of the solution transferred to ice.

Determination of apparent Tₘ was performed using thermal melt. 130 AL of the ice-cold ammonium sulfate precipitates of HAB variants MBP-636 (“WT”), ΔN-HAB1^{T+}, MBP-724, MBP-725, and MBP-726 were pelleted in a microcentrifuge for 5 minutes at 17,000 g.

The supernatant was removed by pipette, discarded, and the pellet resuspended in an equivalent volume of ice-cold CBS⁺⁺⁺. Zeba™ spin desalting columns (Thermo) were equilibrated with CBS⁺⁺⁺ and the resuspended HABs were desalted and placed on ice. Each HAB was diluted to 400 nM concentration in CBS⁺⁺⁺ and aliquoted into PCR tubes at a 50 μL volume. These tubes were distributed into the blocks of a pair of Eppendorf Mastercycler X50i thermocyclers programmed for a thermal gradient as follows (in ° C.): (Machine #1 Gradient 30.0 to 60.0-30.0, 35.4, 39.3, 46.8, 50.6, 54.5, 60.0, Machine #2 Gradient 65.0 to 95.0-65.0, 70.4). The HAB aliquots were held at these temperatures for 30 minutes, followed by cooling to 4° C. in the thermocycler blocks. Once the aliquots had cooled, they were transferred to racks on ice and SO AL of CBSFF⁺⁺⁺ was added, such that each HAB was now at a concentration of 200 nM (10X) in CBSF⁺⁺⁺. The heat treated HAB aliquots were left on ice until the remainder of the labeling reactions were set up, about 1 hour. Labeling reactions were performed, measured, and analyzed as described previously (Steiner et al. 2020).

### Reconstitution of the PYR/HAB Based CID System in a Plate-Based ELISA

Unconjugated 6×His-MBP-PYR1 variants were immobilized to Microlon clear plates (655081) using 100 μL of CBS without pH modification, at a protein concentration of 100 μg mL⁻¹, or 1.57 AM at 4° C. overnight. Wells assigned as no PYR controls received only CBS. Plates were sealed using Microseal B adhesive sealers (BioRad MSB-1001). The following day, the solutions were decanted by flicking and all wells were washed twice with 300 μL of CBS. Blocking was performed using 300 μL of KPL milk diluent/concentrate (SeraCare 5140-0011) diluted 1:20 according to manufacturer's protocol, hereafter referred to as CBSM, incubated at room temperature for 2 hours, during which time all other components of the assays were diluted and assembled in 96 well PCR plates (Biorad HSP9601). Binding reactions were assembled from 88 μL of CBSM, 2 AL of 50× concentrate of ligand, dissolved in anhydrous ethanol for ABA, or anhydrous DMSO for all other compounds, with 10 μL of HAB to be added. Blocked plates were decanted by flicking and were washed twice with 300 μL of CBSM, followed by vigorous tamping on a pad of paper towels. The sole exception to the pre-assembly was biotinylated ΔN-HAB1^{T+}, where 130 AL of ammonium sulfate precipitate was pelleted at 17,000 g for 10 minutes, and the supernatant removed. The pellet was stored on ice until the blocked and washed plates were ready to receive the binding reactions. ΔN-HAB1^{T+} was exchanged into CBSM with 1 mM DTT and 1 mM TCEP (CBSM++) using Zeba™ spin desalting columns (Thermo) were equilibrated with CBSM++. ΔN-HAB1^{T+} was diluted to 10× the final binding reaction concentration and was immediately added to the pre-assembled assays in PCR plates, which were mixed by pipetting and transferred to the washed, blocked Microlon plates. Microlon plates were transferred to a plate shaker (Heidolph Titramax 1000) for 30 minutes at room temperature with shaking at 800 rpm. Plates were decanted by flicking and were washed three times with 300 μL of CBSM, followed by vigorous tamping on a pad of paper towels. Detection was enabled by addition of 100 μL of streptavidin-horseradish peroxidase (Thermo, SA10001), diluted 1:25,000 into CBSM, incubated in the above plate shaker for 10 minutes. This was decanted by flicking and washed three times with 300 AL of CBSM and by vigorous tamping on a pad of paper towels. Bound HAB was detected through addition of 50 μL 1-Step™ Ultra TMB-ELISA Substrate Solution (Thermo, 34028), incubated in the above plate shaker for 15 minutes (PYR1^{M(N90S/H60P)} with ABA, PYR1 ^{WIN-M} and PYR1^{4F-M}. in specificity assays to avoid overdevelopment of positive control), or for 30 minutes (PYR1^{N90S/MANDI} with mandipropamid, and PYR1^{WIN-M} and PYR1^{4F-M} under ligand titrations). TMB development was quenched by addition of 50 μL of 2M sulfuric acid and plates were read at 450 nm using a Biotek Synergy H1 Hybrid multimode plate reader. Estimates of K_{d,eff} were performed using Graphpad Prism 8.4.3 using the specific binding with Hill slope nonlinear regression function. Best fits for K_{d1} and K_{d2} were determined from global nonlinear fitting of the ELISA data using a custom script written in MatLab 2019b.

### Crystallization

PYL2 and ΔN-HAB1^{T+} were expressed in E. coli and purified as described previously (Vaidya et al. 2019). Purified protein was stored at −80° C. in a buffer containing 20 mM HEPES (pH 7.6), 50 mM sodium chloride, 10 mM DTT and 10% glycerol. Purified PYL2 and ΔN-HAB1^{T+} were mixed in 1:1.05 molar reaction and exchange into a buffer containing 20 mM HEPES (pH 7.6), 50 mM sodium chloride, 10 mM dithiothreitol, 5 mM magnesium chloride and 5% glycerol. The proteins were then concentrated to 15 mg/mL and incubated with a 5-fold molar excess of (+)-WIN 55,512 (Cayman Chemical catalog number 10736) for 30 minutes on ice. Crystallization of the PYL2: WIN: ΔN-HAB1^{T+} complex was conducted by sitting drop vapor diffusion at 19° C. Drops were formed by mixing equal volumes of the purified PYL2: WIN: ΔN-HAB1^{T+} complex with well solution containing 100 mM bis-tris propane pH 6.5, 200 mM sodium bromide and 19% (w/v) PEG 3,350. The resulting crystals were flash frozen after passing through a cryoprotection solution consisting of well solution plus 20% glycerol. X-ray diffraction data for each complex were gathered from a single crystal. Diffraction data was collected at 100 K using the LS-CAT ID-21-F beam line at the Advanced Photon Source (Argonne National Labs, Lemont, IL). Diffraction data were indexed, integrated, and scaled using the XDS software package (Kabsch 2010).

### Structure Determination

The PYL2: WIN: ΔN-HABIT complex structure was solved by molecular replacement using a PYL2: Quinabactin: Hab1 complex (PDB ID 4LA7) devoid of ligand and water molecules as the search model to evaluate the initial phases. Phenix. AutoMR (Adams et al. 2010) solved the initial phases and automatically built the majority of residues for both complexes. The resulting models were completed through iterative rounds of manual model building in Coot (Emsley and Cowtan 2004) and refinement with Phenix.refine using translational libration screw-motion (TLS) and individual atomic displacement parameters. A Phenix topology file for the (+)-WIN 55,512-2 ligand was generated using the PRODRG server (http://davapc1.bioch.dundee.ac.uk/cgi-bin/prodrg/) (Schüttelkopf and van Aalten 2004). Geometry of the final structures were validated using Molprobity (Davis et al. 2007). Data collection and refinement statistics for the final PYL2: WIN: ΔN-HAB1^{T+} model are listed in Table 4 and the coordinates for the structure deposited in the Protein Data Bank, PDB ID 7MWN.

### Screens for Organophosphate Sensor

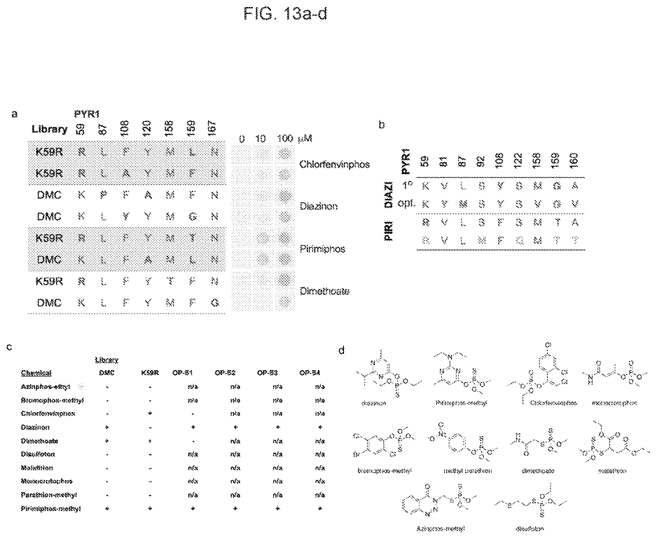

MAV99 harboring either the K59R-NNK or DMC libraries were subjected to negative selections to remove constitutively active receptors by plating onto synthetic dextrose (SD) medium lacking leucine and tryptophan, and supplemented with 0.1% FOA (SD,-Leu,-Trp,+FOA). Surviving cells were collected and used in subsequent positive selections for ligand-responsive receptors on SD,-Leu,-Trp,-Ura media supplemented with a specific test chemical (100 μM) for responsiveness to any of the 10-member panel of organophosphate compounds: diazinon, pirimiphos-methyl, azinphos-ethyl, dimethoate, chlorfenvinphos, disulfoton, parathion-methyl, bromophos-methyl, malathion, and monocrotophos; all compounds were purchased form (Sigma-Aldrich). Isolated colonies were tested by regrowth on ligand-containing media and by dose response using X-gal staining and Sanger sequenced (see FIG. 13a-d).

### Organophosphate Sensor Optimization

Recombination-based mutagenesis of hit receptors was used to generate subsequent molecular diversity for selection-based optimization experiments, using the same general strategy across all scaffolds utilized. In the case of the PYR1-derived organophosphate sensors, plasmids for ligand-responsive mutants were diversified for subsequent optimization by recombination with one another, the parental DMC, and previously described PYR1 and PYR1^{K59R} SSM libraries that harboring saturating mutations in 39 and 25 sites respectively (Park et al. 2015; Mosquna et al. 2011). These DNAs were mixed in a 40:20:20:20 ratio (hit mutants/DMC/SSM/K59R-SSM), and the PYR1 coding sequence PCR amplified from the pool, and subjected to recombination-based mutagenesis using nucleotide excision and exchange technology (NexT) (Müller et al. 2005). The resulting shuffled DNA fragments were cloned into pBD, transformed into E. coli, colonies collected, and plasmids extracted. In total, four optimization libraries (OP-S1, OP-S2, OP-S3, OP-S4) were constructed throughout the optimization process by adding newly isolated hit receptors at each stage to the DMC/SSM/K59R-SSM DNA library pool used for NEXT mutagenesis. The initial organophosphate receptors for diazinon, pirimiphos-methyl, chlorfenvinphos, and dimethoate were combined to create OP-S1, which was constructed using the following PYR1 mutants: K59R/M158T, K59R/M158V, K59R/F159T, K59R/F159C, K59R/F159I, K59R/Y120A, Y120A/F159L, Y120A/F159T, L87P/Y120A, Y120A/F159T, L87M/Y159G, F108Y/F159G, K59R/F108A. K59R/F159L, K59R/1135R/T162W and N167G. OP-S2 was constructed using PYR1-S16P/S29G/Q36R/K59R/S92M/F159 and PYR1-F108Y/D154G/F159G/A160V. The OP-S3 library used the following mutants: PYR1-K59R/S92M/F159T/V174A and PYR1-L87M/F108Y/F159G/A160V. Finally, OP-S4 used PYR1-K59R/S92M/S122Q/E130G/F159T/V174A and PYR1 V81Y/L87M/F108Y/F159G/A160V.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference for the purpose for which it was cited to the same extent as if each reference was individually incorporated by reference.

TABLE 1
TABLES
Design of the PYR1 double mutant pocket library.
This library encodes a total of 42,743 double
mutants, of which 35,822 are distant mutations
(separated by at least eight amino acids) and
6,921 are proximate mutations (separated by
fewer than eight amino acids).

| Position | WT | Amino Acids |
|---|---|---|
| 59 | K | ADEFGHIKLMNQRSTVWY |
| 62 | I | ILRVY |
| 81 | V | ILMRTVY |
| 83 | V | ADEFGHIKLMNQRSTVWY |
| 87 | L | AFGILMVWY |
| 89 | A | ADEFGHIKLMNQRSTVWY |
| 92 | S | ADEFGHIKLMNQRSTVWY |
| 94 | E | ADEFGHIKLMNQRSTVWY |
| 108 | F | ADEFGHIKLMNQRSTVWY |
| 110 | I | AFIKLMRTVW |
| 117 | L | ADEFGHIKLMNQRSTVWY |
| 120 | Y | ADEFGHIKLMNQRSTVWY |
| 122 | S | ADEFGHIKLMNQRSTVWY |
| 141 | E | ADEFGHIKLMNQRSTVWY |
| 159 | F | ADEFGHIKLMNQRSTVWY |
| 160 | A | ADEFGHIKLMNQRSTVWY |
| 163 | V | ADEFGHIKLMNQRSTVWY |
| 164 | V | ADEFGHIKLMNQRSTVWY |
| 167 | N | ADEFGHIKLMNQRSTVWY |

TABLE 2
Double mutant library coverage statistics. Composition of the
proximate and distant double mutant libraries is shown. Library
encoded variants are any for which all mutations in a variant
were encoded in the library. Quintuple and higher mutants present
at very low frequencies are omitted for clarity. Other variants
are those with at least one mutation not encoded. Coverage is
shown for the target set of double mutants for each library (6,921
double mutants for L0007 and 35,822 for L0008).

| | Proximate | Distant |
|---|---|---|
| | (L0007) | (L0008) |
| Quality filtered reads | 1,397,492 | 5,461,353 |
| Library composition (%) | | |
| Wild-type | 34.29 | 9.29 |
| Library encoded | | |
| single | 5.45 | 41.68 |
| double | 50.39 | 36.94 |
| triple | 0.63 | 3.69 |
| quadruple | 1.44 | 0.17 |
| Other | 7.77 | 8.22 |
| Observed/Encoded | 6920/6921 | 35,748/35,822 |
| double mutants | | |
| Coverage (%) | 99.99 | 99.79 |

TABLE 3
Protein function and stability parameters for HAB1-636 (WT), and the designs
ΔN-HAB1^{T+} and 724-726 from 2 independent expression and purification
experiments, with three technical replicates for each parameter. Phosphatase activity is
reported as percentage of the activity found for HAB1-636 using 42.86 mM p-NPP, with error
reported as 1 SEM. Thermal stability, as measured using T_{m, app}, is reported
in ° C. and binding constant K_{D2} of each HAB1 variant for PYR1^{M} is
reported in nM. Error for both T_{m, app} and K_{D2} are
reported as the 95% confidence interval, where undef indicates that Graphpad did not
report an upper limit.

| | 6xHis-MBP-ΔN-HAB1 variant - Biorep 1 | | | | |
|---|---|---|---|---|---|
| | WT (636) | T+ (723) | 724 | 725 | 726 |
| Phosphatase | 100 ± 0.4 | 0.21 ± 0.02 | 0.22 ± 0.005 | 0.25 ± 0.01 | 0.27 ± 0.005 |
| Activity | | | | | |
| (% of 636 | | | | | |
| at 50 mM | | | | | |
| p-NPP) | | | | | |
| Tm, app | 44.8 | 54.4 | 54.1 | 50.7 | 50.5 |
| (° C.) | [43.6, undef] | [54.06, 54.72] | [53.73, 54.50] | [50.45, 51.18] | [50.00, 51.03] |
| KD2 | 2.53 | 2.13 | 3.33 | 1.65 | 2.37 |
| (nM) | [2.31, 2.76] | [1.94, 2.32] | [3.10, 3.58] | [1.52, 1.76] | [2.24, 2.51] |

| | 6xHis-MBP-ΔN-HAB1 variant - Biorep 2 | | | | |
|---|---|---|---|---|---|
| | WT (636) | T+ (723) | 724 | 725 | 726 |
| Phosphatase | 100 ± 0.8 | 0.04 ± 0.005 | 0.01 ± 0.001 | 0.003 ± 0.004 | 0.03 ± 0.01 |
| Activity | | | | | |
| (% of 636 | | | | | |
| at 50 mM | | | | | |
| p-NPP) | | | | | |
| Tm, app | 38.1 | 44.2 | 44.8 | 45.5 | 44.0 |
| (° C.) | [38.03, 38.21] | [43.98, 44.52] | [44.53, 44.99] | [45.16, 45.94] | [43.36, |
| | | | | | 44.66] |
| KD2 | 5.01 | 4.76 | 6.49 | 4.66 | 6.3 |
| (nM) | [4.47, 5.62] | [4.18, 5.42] | [5.83, 7.23] | [3.55, 6.01] | [5.15, |
| | | | | | 7.71] |

TABLE 4
Data collection and refinement statistics

| | PYL2:WIN:ΔN-HAB1^{T+} |
|---|---|
| Data collection | |
| Space group | P 21 21 21 |
| Cell dimensions | |
| a, b, c (Å) | 61.33, 69.23, 142.82 |
| α, δ, γ (°) | 90, 90, 90 |
| Resolution (Å) | 50-1.902 |
| | (1.97-1.902) |
| Total Reflections | 351,970 |
| | (34628) |
| Unique Reflections | 48,429 |
| | (4,740) |
| R_{merge} | 0.060 |
| | (1.270) |
| Rₘₑₐₛ | 0.065 |
| | (1.366) |
| Rₚᵢₘ | 0.024 |
| | (0.499) |
| CC½ | 0.999 |
| | (0.756) |
| CC* | 1.000 |
| | (0.928) |
| I/σI | 16.7 |
| | (1.7) |
| Completeness (%) | 99.6 |
| | (98.8) |
| Redundancy | 7.3 |
| | (57.3) |
| Refinement | |
| Resolution (Å) | 38.61-1.902 |
| | (1.97-1.902) |
| No. reflections | 48,408 |
| | (4,740) |
| R_{cryst} | 0.185 |
| | (0.326) |
| R_{free} | 0.211 |
| | (0.333) |
| No. atoms | 3967 |
| Protein | 3685 |
| Ligand/ion | 63 |
| Water | 219 |
| B-factors | |
| Protein | 53.3 |
| Ligand/ion | 46.1 |
| Water | 50.2 |
| R.m.s. deviations | |
| Bond lengths (Å) | 0.007 |
| Bond angles (°) | 0.770 |
| Ramachandran Stats | |
| Favored | 98.1% |
| Allowed | 1.9% |

Values in parentheses are for highest-resolution shell.
A single crystal was used for each structure determination.

TABLE 5
Global parameters for the PYR1^{M}/ABA/ΔN-HAB1^{T+} system
in the context of a plate-based ELISA assay for three
independent biological replicates.

| | Biorep 1 | Biorep 2 | Biorep 3 |
|---|---|---|---|
| Aₘₐₓ [AU 450 nm] | 2 | 2.8 | 2 |
| K_{D1} [nM] | 820 | 780 | 2090 |
| K_{D2} [nM] | 8 | 4 | 2 |

TABLE 6
PYR1 K59R SSM library statistics.

| | Library | | |
|---|---|---|---|
| | PYR1 KS9R SSM Library | | |
| | Sub-Library | | |
| | PYR1_KS9R-Tile1 | PYR1_KS9R-Tile2 | PYR1_K59R-Tile3 |
| | Amino Acids: | | |
| | M1-Y58, F61-S64 | C65-R128 | F129-T191 |
| Total QC Reads (millions) | 0.36 | 0.40 | 0.38 |
| Wild-Type Sequences | 32.0% | 36.0% | 29.0% |
| Sequences with exactly 1 | 46.0% | 47.0% | 51.0% |
| nonsynonymous mutation: | | | |
| Sequences with more than 1 | 22.0% | 17.0% | 20.0% |
| nonsynonymous mutations: | | | |
| Coverage of all possible single | 100% | 100% | 99.9% |
| nonsynonymous mutations: | (1,260/1,260) | (1,280/1,280) | (1,259/1,260) |

| | |
|---|---|
| Coverage of all possible single | 99.9% |
| nonsynonymous mutations (total): | (3,799/3,800) |

TABLE 7
On and off-target responses of PYR1^{DIAZI} and PYR1^{PIRI}.

PYR1-DIAZI

| | Pirimiphos | Diazinon | Azinphos | Parathion | Coumaphos | Paraoxon |
|---|---|---|---|---|---|---|
| Bottom | 14673 | 17196 | 19538 | 31717 | 23886 | 26788 |
| Top | 88148 | 103999 | 91584 | 50592 | 61154 | 74641 |
| EC50 | 17401 | 43.09 | 1118 | 333.2 | 772.5 | 1235 |
| EC50 - 95% CI | 9830 to 33274 | 12.51 to 142.1 | 523.6 to 2331 | ??? to +infinity | 0.2688 to 18821 | 0.4301 to 24256 |
| R squared | 0.938 | 0.8666 | 0.9159 | 0.1025 | 0.3191 | 0.4377 |

PYR1-PIRI

| | Pirimiphos | Diazinon | Azinphos | Parathion | Coumaphos | Paraoxon |
|---|---|---|---|---|---|---|
| Bottom | 12745 | 19123 | 15284 | 11885 | 13267 | 12031 |
| Top | 105951 | 109997 | 104767 | 40075 | 36298 | 38379 |
| EC50 | 20.04 | 1014 | 26941 | 2593 | 2676 | 2368 |
| EC50 - 95% CI | 8.570 to 51.16 | 31.84 to 9805 | 6992 to +infinity | 70.80 to 75281 | 4.299 to +infinity | 12.70 to +infinity |
| R squared | 0.95 | 0.5161 | 0.7363 | 0.364 | 0.2058 | 0.2735 |

TABLE 8
Primers used in this study.
Table discloses SEQ ID NOS 38-110, respectively,
in order of appearance.

| Name | Sequence |
|---|---|
| PJS-P2062 | TTGGAATTTAATCGCGGCCT |
| PJS-P2063 | TTCAACGGGAAACGTCTTGCT |
| PJS-P2079 | CATAATGGGATGGAGTCCAGCTCGTAGACGCT |
| PJS-P2080 | ACGAGCTGGACTCCATCCCATTATGGGGAACT |
| PJS-P2081 | ACGCTTAGACAATTCATCTTTAATACGTTCAATCTC |
| PJS-P2082 | TATTAAAGATGAATTGTCTAAGCGTAACACCGGGGA |
| PJS-P2112 | CTCGTATGAGACCGTGTTACAACCAATTAACCAATTC |
| PJS-P2113 | TTGACCAGATGAGACCACGGTTATCCACAGAATCA |
| PJS-P2114 | GGATAACCGTGGTCTCATCTGGTCAATCGTACGAC |
| PJS-P2115 | TGGTTGTAACACGGTCTCATACGAGCCATAGCTTCAG |
| PJS-P2116 | ATACCGCGAGATCCACGCTCACCGGCTCCA |
| PJS-P2117 | TCAGCGGAGATCTTTTGGTTTTGGGAGAGTAG |
| PJS-P2118 | CCCAAAACCAAAAGATCTCCGCTGACTAGGGC |
| PJS-P2119 | CGTTTCACTTTCGGTCTCACAGACGAGTTCCGGAG |
| PJS-P2120 | CGTCTGTGAGACCGAAAGTGAAACGTGATTTCATG |
| PJS-P2121 | GAGTTACGTGAGACCTATAAACGCAGAAAGGCCCA |
| PJS-P2122 | TGCGTTTATAGGTCTCACGTAACTCCGGTGACGGA |
| PJS-P2123 | GGTGAGCGTGGATCTCGCGGTATCATTGCA |
| PJS-P2124 | GCGTGTTACAACCAATTAACCAATTCT |
| PJS-P2125 | TGAGGTCTCATACGAGCCATA |
| PJS-P2243 | GTTTCCCTGGATTGAGACAG |
| PJS-P2244 | CTTGCACTGCAGAAGGGATC |
| PJS-P2245 | AATCAAGAGGTATGTGAGAT |
| PJS-P2246 | CTGTCTCAATCCAGGGAAACGCGTCTGAGATGGAGGATGCGTTTGCAGTAGTGC |
| PJS-P2247 | TGCTGCCTCCGTGACCCGCGTAGACTCCGAAAAAATGGC |
| PJS-P2248 | CGCGGGTCACGGAGGCAGCAAAGTGGCAGATTACTGTCG |
| PJS-P2249 | ACAATTCGCTACTACAATGT |
| PJS-P2250 | CGTGACCCGCGTAGACTCCGAAAAAATGGC |
| PJS-P2251 | TCGGAGTCTACGCGGGTCACGGAGGCCACA |
| PJS-P2330 | CATATGCCTTCGGAGTTAACACCAGGTGAACGATCGGAACTAAAAAACTCA |
| PJS-P2331 | ATGCGCGTCGGATGCACGCGCGACTATATCGTCATCAGTGGAATGCCGGCGTTCTACATCAACGGAAAGA |
| PJS-P2332 | GGTAACTCGGAGGATGATACTCGTGTTGGTGTTGATACGGTTGTGAAGCTTAATTTG |
| PJS-P2334 | CGATTCGACAAACCACAAACATACCAACCGTTCATCAAATCCTGCTCCGTC |
| PJS-P2335 | AACTCGGAGGATGATACTCGTATGGCTATTGATACGGTTGTGAAGCTTAATTTG |
| PJS-P2336 | CGATTCGACAAACCACAAACATACAATCCGTTCATCAAATCCTGCTCCGTC |
| PJS-P2337 | CTCAGGTGACGTGAGATCCGGCTGCTA |
| PJS-P2338 | GGCATACCTCCCCCGCCGCCAGTCTGCGCGTCT |
| PJS-P2339 | GGCGGCGGGGGAGGTATGCCTTCGGAGTTAACA |
| PJS-P2340 | CGGATCTCACGTCACCTGAGAACCACT |
| PJS-P2395 | GTTTGTGGTTTGTCGAATCG |
| PJS-P2401 | CACACATCAACTCGGTCCAGGAAGCTGTTCATCA |
| PJS-P2402 | CGAGTATCATCCTCCGAGTT |
| PJS-P2403 | TACCATTCGTCAGCCTTTGTTCGCCTCCGATGATAC |
| PJS-P2404 | ACAAAGGCTGACGAATGGTAAATCCGTTACGACGGT |
| PJS-P2405 | GTATCACCAACCATACGAGTATCATCCTCCGA |
| PJS-P2406 | ACTCGTATGGTTGGTGATACGGTTGTGAAGCT |
| SW-022 | CCTCATCAAGATTGCTTTATGCTAGCTCACGTCACCTGAGAACCACTT |
| SW-023 | GCTAGCATAAAGCAATCTTGATGAGGA |
| SW-024 | CAGACAAGCTGTGACCGTCTTCCCGGCGTTCAGGGTAATATATTTTAACCG |
| SW-025 | GCCAGGCGCCTTTATATCATAT |
| SW-026 | CGCTCATCTTGTGCATGAGACTTGCGAAACCCTATGCTCTGT |
| SW-027 | CGGCGGAGGAGTGACCATCCGAATTCGTCGACGAGCTCACGCGTTC |
| SW-028 | TATTAATTTAGTGTGTGTATTTGTGT |
| SW-029 | ATACACACACTAAATTAATAGCGCGCATGAGTAAAGGAGAAGAACTTTT |
| SW-030 | TTTGTATAGTTCATCCATGCCA |
| SW-035 | GGAGAAAATACCGCATCAGGCCGCGGAATGGAAGGTCGGGATGAGC |
| SW-036 | ATAAAGCAGCCGCTACCAAACA |
| SW-042 | TGGTGGGGGTTCATCTGGTGCTAGCATGCCTTCGGGGTTAACACCAG |
| SW-043 | CTCATCAAGATTGCTTTATGAGATCTTCACGTCACCTGAGAACCACTT |
| SW-422 | ACACAAAGATACATACGGGAGGATCCATGCCTTCGGAGTTAACACCAG |
| SW-423 | CCTCATCAAGATTGCTTTATGCTAGCTCACGTCACCTGAGAACCACTTC |
| SW-646 | ACTAAAGGGAACAAAAGCTGGAGCTCCTTGCCAACAGGGAGTTCT |
| SW-645 | CTTCCAGAGTGAATACCATGCGGCCGCTTAGATTAGATTGCTATGCT |
| PYR1-F108Y-TAT | GACGACGAACGGAGAGTTACCGGATATAGTATCATCGGAGGCGAACATAGG |
| PYR1-Y120H-CAT | GGAGGCGAACATAGGCTGACGAATCATAAATCCGTTACGACGGTGCATCGG |
| PYR1-A160G-GGT | TCGGAGGATGATACTCGTATGTTTGGTGATACGGTTGTGAAGCTTAATTTG |
| pET29B+ backbone_fwd | ACGTACGTGAGATCCGGCTGCTAACAAAG |
| pET29B+ backbone_rev | TGCTATTCATGGTATATCTCCTTCTTAAAGTTAAACAAAATTATTTC |
| met-asn-His-MBP-TEV_fwd | GAGATATACCATGAATAGCAGCCATCATCATCATCATCAC |
| met-asn-His-MBP-TEV_rev | CGCTGCGACCTCCCCCGCCGCCAGTCTG |
| dN-HAB1_fwd | CGGCGGGGGAGGTCGCAGCGTCTACGAG |
| dN-HAB1_rev | CAGCCGGATCTCACGTACGTGTTTTAAACTTGC |
| HABstarR199AD204A | GGAACTGTCTCAATCCAGGGAAACGCGTCTGAGATGGAGGCGGCGTTTGCAGTAAGCCCCCACTTT |

TABLE 9
Plasmids used in this study.

| Plasmid | Description | Source |
|---|---|---|
| pYTK047 | Yeast MoClo Toolkit GFP dropout (Cam^{R}) | Addgene (Kit |
| | | #1000000061) |
| pYTK084 | Yeast MoClo Toolkit Kan^{R} ColE1 origin | Addgene (Kit |
| | | #1000000061) |
| pMAL-C5G_ccDockerin | Expression of MBP-ccDockerin | (Kowalsky and |
| | | Whitehead |
| | | 2016) |
| pBD-PYR1-BbvCI | GAL4-DBD-PYR1 fusion expression for Y2H | (Medina- |
| | | Cucurella et al. |
| | | 2019) |
| pJS624 | Yeast surface display vector of PYR^{MANDI} (N90S). | (Steiner et al. |
| | | 2020) |
| pJS636 | E. coli expression of 6 × His-ΔN-HAB1 (C186S, C274S) | This study |
| | (pET derivative, Kan^{R}) | |
| pJS636-MBP | E. coli expression of 6 × His-MBP-ΔN-HAB1 (C186S, C274S) | This study |
| | (pET derivative, Kan^{R}) | |
| pJS637 | YSD of PYR1 (H60P, N90S); PYR1^{M} | (Steiner et al |
| | | 2020) |
| pJS645 | PYR1 cassette for mutagenesis | This study |
| pJS646 | PYR1 cassette acceptor for Y2H | This study |
| pJS647 | PYR1 cassette with BbvCI site | This study |
| pJS678 | E. coli expression of 6 × His-MBP-ΔN-HAB1 (C186S, D199A, | This study |
| | D204A, C274S) (pET derivative, Kan^{R}) | |
| pJS723 | E. coli expression of 6 × His-MBP-ΔN-HAB1 (C186S, D199A, D204A, | This study |
| | S209V, H248S, F260E, C274S, D287K, S292N, S342A, M358I, N377A, | |
| | R422E, N439S, M483K) (pET derivative, Kan^{R}) | |
| pJS724 | E. coli expression of 6 × His-MBP-ΔN-HAB1 (C186S, D199A, S209V, | This study |
| | D243A, H248S, F260E, C274S, D287K, S292N, S342A, M358I, N377A, | |
| | R422E, N439S, M483K) (pET derivative, Kan^{R}) | |
| pJS725 | E. coli expression of 6 × His-MBP-ΔN-HAB1 (C186S, D199A, D204A, | This study |
| | S209V, D287E, S292N, S342A, N377A, N439S) (pET derivative, Kan^{R}) | |
| pJS726 | E. coli expression of 6 × His-MBP-ΔN-HAB1 (C186S, D199A, S209V, | This study |
| | D243A, D287E, S292N, S342A, N377A, N439S) (pET derivative, Kan^{R}) | |
| pJS753 | YSD of PYR1-WIN (K59Q, H60P, N90S, F159A, A160I) | This study |
| pJS754 | YSD of PYR1-JWH-A (K59N, H60P, N90S, Y120H, A160G) | This study |
| pJS755 | 6xHis-MBP-PYR1-H60P-N90S (pET derivative, Kan^{R}) | This study |
| pJS756 | 6xHis-MBP-PYR1-Mandi (pET derivative, Kan^{R}) | This study |
| pJS794 | T7-MBP-PYR1-WIN (Y23H, D26G, K59Q, H60P, N90S, F159A, A160I) | This study |
| | (pET derivative, Kan^{R}) | |
| pJS796 | T7-MBP-PYR1-4FB (H60P, N90S, H115Q, Y120G, F159V, A160G) | This study |
| | (pET derivative, Kan^{R}) | |
| pRS426 | S. cerevisiae/E. coli shuttle vector, 2 micron, URA | ATCC |
| pSW004 | pRS426, TEF1p-SV40-Z4DBD-PYR1-CYC1t, PGK1p-VP64-HAB1-PGK1t | This study |
| pSW010 | p415GPD, HDup-Z4(4)-CYC1core-GFP-CYC1t-HDdown | This study |
| pSW143 | pRS426, TEF1p-SmBit-linker-PYR1-CYC1t, TEF1p-LgBit-linker-HAB1- | This study |
| | PGK1t | |
| pIW15 | pRS426, PGK1p-GFP-PGK1t | This study |
| pIW156 | p415GPD | (Mumberg, |
| | | Müller, and |
| | | Funk 1995) |

TABLE 10
gBlocks used in this study.

| | | SEQ ID |
|---|---|---|
| Name | Sequence | NO: |
| PJS-G0023 | CTGTCTCAATCCAGGGAAACGCGTCTGAGATGGAGGCGGCGTTTGCAGTAGTGCCCCACTTTTTAAA | 111 |
| | GTTACCTATCAAGATGTTGATGGGGGACCACGAGGGGATGAGTCCTTCTCTGACGCACTTAACGGGC | |
| | CATTTTTTCGGAGTCTACGATGGTCACGGAGGCAGCAAAGTGGCAGATTACTGTCGTGACCGCCTTC | |
| | ACGAAGCCCTGGCCGAAGAGATTGAACGTATTAAAGATGAATTGAGCAAGCGTAACACCGGGGAGGG | |
| | ACGCCAGGTGCAGTGGAAAAAGGTCTTCACCAACTGTTTCCTGACTGTTGATGGAGAGATTGAAGGA | |
| | AAGATCGGTCGCGCAGTGGTAGGTAGCTCCGACAAGGTGTTGGAAGCGGTCGCGTCAGAAACTGTGG | |
| | GGTCGACAGCTGTGGTTGCCTTAGTGTGTAGTTCTCACATTGTAGTAGCGAATTGT | |
| PJS-G0024 | ACATTGTAGTAGCGAATTGTGGTGATTCACGCGCGGTGCTGTTTCGTGGCAAGGAGGCTATTCCCCT | 112 |
| | GAGCGTTGACCACAAACCGGACCGTGAGGACGAATATGCCCGCATCGAGGCGGCTGGTGGCAAGGTA | |
| | ATCCAATGGCAGGGTGCGCGCGTATTCGGGGTCCTGGCAATGTCACGTAGCATTGGTGACCGCTATT | |
| | TGAAACCATACGTTATCCCGGAGCCTGAGGTTACCTTCATGCCCCGCAGTGAAGAAGACGAATGTTT | |
| | AATTTTGGCCAGTGACGGGCTGTGGGATGTTATGAGCAATCAAGAGGTATGTGAGATCGCCCGTCGT | |
| | CGTATCTTAATGTGGCACAAGAAAAATGGGGCTCCACCTCTTGCTGAACGCGGCAAAGGGATTGACC | |
| | CCGCTTGTCAGGCTGCCGCAGACTATCTGTCAAAACTTGCACTGCAGAAGGGATC | |
| PJS-G0025 | CTGTCTCAATCCAGGGAAACGCGTCTGAGATGGAGGCGGCGTTTGCAGTAGTGCCCCACTTTTTAAA | 113 |
| | GTTACCTATCAAGATGTTGATGGGGGACCACGAGGGGATGAGTCCTTCTCTGACGCACTTAACGGGC | |
| | CATTTTTTCGGAGTCTACGATGGTCACGGAGGCCACAAAGTGGCAGATTACTGTCGTGACCGCCTTC | |
| | ACTTTGCCCTGGCCGAAGAGATTGAACGTATTAAAGATGAATTGAGCAAGCGTAACACCGGGGAGGG | |
| | ACGCCAGGTGCAGTGGGAAAAGGTCTTCACCAACTGTTTCCTGACTGTTGATGGAGAGATTGAAGGA | |
| | AAGATCGGTCGCGCAGTGGTAGGTAGCTCCGACAAGGTGTTGGAAGCGGTCGCGTCAGAAACTGTGG | |
| | GGTCGACAGCTGTGGTTGCCTTAGTGTGTAGTTCTCACATTGTAGTAGCGAATTGT | |
| PJS-G0026 | ACATTGTAGTAGCGAATTGTGGTGATTCACGCGCGGTGCTGTTTCGTGGCAAGGAGGCTATGCCCCT | 114 |
| | GAGCGTTGACCACAAACCGGACCGTGAGGACGAATATGCCCGCATCGAGGCGGCTGGTGGCAAGGTA | |
| | ATCCAATGGCAGGGTGCGCGCGTATTCGGGGTCCTGGCAATGTCACGTAGCATTGGTGACCGCTATT | |
| | TGAAACCATACGTTATCCCGGAGCCTGAGGTTACCTTCATGCCCCGCAGTCGCGAAGACGAATGTTT | |
| | AATTTTGGCCAGTGACGGGCTGTGGGATGTTATGAGCAATCAAGAGGTATGTGAGAT | |
| SW-G001 | ATGCCAAAAAAGAAGAGAAAGGTCTCCCGAGGTGGAGCCGCTGGAGGCACTAGACCGTATGCCTGCCCTGTAGA | 115 |
| | GTCCTGTGATAGACGTTTTAGCAGACATGCCAACCTTACACGTCACATCAGAATTCACACAGGGCAAAAACCTT | |
| | TTCAGTGCAGAATTTGTATGAGAAATTTCAGCAGGAATGCGAACCTAGTAAGACATATTAGGACGCACACAGGT | |
| | TCCCAGAAGCCTTTTCAATGTCGTATCTGTATGCGTAACTTCTCTAGGAAGGCAGACCTGAGAAGGCATATCAG | |
| | AACGCACACAGGAGAGAAACCATTTGCGTGCGACATCTGCGGTAGGAAGTTCGCCCGTAAAGGCGACTTAAAAA | |
| | GGCACACAAAGATACATACGGGA | |
| SW-G002 | ATGCCAAAAAAGAAGAGAAAGGTCTCCCGAGGTGGAGCCGACGCCCTGGACGATTTCGACTTGGATA | 116 |
| | TGCTTGGTAGTGACGCGCTAGATGATTTTGATTTGGATATGTTAGGATCTGACGCCCTAGACGACTT | |
| | CGATCTGGATATGCTTGGGTCCGACGCGCTGGATGATTTCGACCTGGACATGTTA | |
| SW-G003 | GCACAAGATGAGCGGCGGAGGAGGAAAACGTGCGGCGGAGGAGGTATAGCAGCGGCGGAGGAGTTAT | 117 |
| | CGGAGCGGCGGAGGAGGGTTCTCTGTATCATGTCGAAGTTCGTTATTGGACTCATTCAACAGAAGTA | |
| | GCGGCGGAGGAGTCACTAGAGCGGCGGAGGAGAGTGTGTTGCGGCGGAGGAGAGCAACTAGCGGGGG | |
| | AGGAGCAGTGTGCGTAACTTCCTTAGCTGAAGTGCTGGGAACGCTCAATCAAAGACGCGGCGGAGGA | |
| | GGCTACAAGGCGGCGGAGGAGTAGTGAGAGCGGCGGAGGAGCATACAAGGCGGCGGAGGAGATCGTT | |
| | GGTGCATCATCCAGTGCGGCGGAGGAGATCAGCGGCGGAGGAGTTCAGCGGCGGAGGAGACAAGCGG | |
| | CGGAGGAGTGACCATCCGC | |
| SW-G004 | CATAGCAATCTAATCTAAGCCTAGGATGGTGACCGGCTACCGGCTGTTCGAGGAGATTCTCGGGAGT | 118 |
| | TCCGGTGGTGGCGGGAGCGGAGGTGGAGGCTCGAGCGGTGCTA | |
| SW-G005 | ACCGCTCGAGCCTCCACCTCCGCTCCCGCCACCACCGGAACTCCCACTGTTGATGGTTACTCGGAAC | 119 |
| | AGCATGGAGCCGTCGGGGGTGATCAGGCGCTCGTCGATAATTTTGTTGCCGTTCCACAGGGTCCCTG | |
| | TTACAGTGATCTTTTTGCCGTCGAACACGGCGATGCCTTCATACGGCCGTCCGAAATAGTTCAGCAT | |
| | GTTCGGCGTAACCCCGTCGATTACCAGTGTGCCATAGGGCAGGATCACCTTAAAGTGATGATCATCC | |
| | ACAGGGTACACCACCTTAAACACCTCTTCGATCTGGGCCATTTGGTCGGCGCTCAGACCTTCATACG | |
| | GGATGATGACATGGATGTCGATCTTCAGGGCATTTTCACCGCTCCGGACAATCCTTTGGATCGGAGT | |
| | TACGGACACGGCGAGATTCTGCAGCAAACTGGACACACCTCCCTGTTCAAGGACTTGGTCCAGGTTG | |
| | TAGGCGGCTGTCTGTTCCCAGTCCCCAACGAAATCTTCGAGTGTGAAGACCATGCGGCCGCCACCGC | |
| | GGTGATCTGTAA | |

### REFERENCES CITED BY NUMBER

1. Glasgow, A. A. et al. Computational design of a modular protein sense-response system. Science 366, 1024-1028 (2019).
2. Taylor, N. D. et al. Engineering an allosteric transcription factor to respond to new ligands. Nature Methods vol. 13 177-183 (2016).
3. Urban, D. J. & Roth, B. L. DREADDs (designer receptors exclusively activated by designer drugs): chemogenetic tools with therapeutic utility. Annu. Rev. Pharmacol. Toxicol. 55, 399-417 (2015).
4. Tinberg, C. E. et al. Computational design of ligand-binding proteins with high affinity and selectivity. Nature 501, 212-216 (2013).
5. Polizzi, N. F. & DeGrado, W. F. A defined structural unit enables de novo design of small-molecule-binding proteins. Science vol. 369 1227-1233 (2020).
6. Bick, M. J. et al. Computational design of environmental sensors for the potent opioid fentanyl. Elife 6, (2017).
7. Stanton, B. Z., Chory, E. J. & Crabtree, G. R. Chemically induced proximity in biology and medicine. Science 359, (2018).
8. Park, S.-Y. et al. Agrochemical control of plant water use using engineered abscisic acid receptors. Nature 520, 545-548 (2015).
9. Medina-Cucurella, A. V. et al. User-defined single pot mutagenesis using unamplified oligo pools. Protein Eng. Des. Sel. 32, 41-45 (2019).
10. Ma, Y. et al. Regulators of PP2C phosphatase activity function as abscisic acid sensors. Science 324, 1064-1068 (2009)
11. Szostkiewicz, I. et al. Closely related receptor complexes differ in their ABA selectivity and sensitivity. Plant J. 61, 25-35 (2010).
12. Wrenbeck, E. E. et al. Plasmid-based one-pot saturation mutagenesis. Nat. Methods 13, 928-930 (2016).
13. White, C. M. The Pharmacologic and Clinical Effects of Illicit Synthetic Cannabinoids. J. Clin. Pharmacol. 57, 297-304 (2017).
14. Tait, R. J., Caldicott, D., Mountain, D., Hill, S. L. & Lenton, S. A systematic review of adverse events arising from the use of synthetic cannabinoids and their associated treatment. Clin. Toxicol. 54, 1-13 (2016).
15. Trecki, J., Gerona, R. R. & Schwartz, M. D. Synthetic Cannabinoid-Related Illnesses and Deaths. N. Engl. J. Med. 373, 103-107 (2015).
16. Backman, T. W. H., Cao, Y. & Girke, T. ChemMine tools: an online service for analyzing and clustering small molecules. Nucleic Acids Res. 39, W486-91 (2011).
17. Steiner, P. J., Bedewitz, M. A., Medina-Cucurella, A. V., Cutler, S. R. & Whitehead, T. A. A yeast surface display platform for plant hormone receptors: Toward directed evolution of new biosensors. AIChE J. 66, (2020).
18. Melcher, K. et al. A gate-latch-lock mechanism for hormone signalling by abscisic acid receptors. Nature 462, 602-608 (2009).
19. Miyazono, K.-I. et al. Structural basis of abscisic acid signalling. Nature 462, 609-614 (2009).
20. Yin, P. et al. Structural insights into the mechanism of abscisic acid signaling by PYL proteins. Nat. Struct. Mol. Biol. 16, 1230 (2009).
21. Xing, C. et al. Cryo-EM Structure of the Human Cannabinoid Receptor CB2-Gi Signaling Complex. Cell vol. 180 645-654.e13 (2020).
22. Dixon, A. S. et al. NanoLuc Complementation Reporter Optimized for Accurate Measurement of Protein Interactions in Cells. ACS Chem. Biol. 11, 400-408 (2016).
23. Hasegawa, K. et al. Postmortem distribution of MAB-CHMINACA in body fluids and solid tissues of a human cadaver. Forensic Toxicol. 33, 380-387 (2015).
24. New Psychoactive Substances Discovery Dashboard. Synthetic Cannabinoids Trend Report https://www.npsdiscovery.org/wp-content/uploads/2021/04/2021-Q1_Synthetic-Cannabinoids_Trend-Report.pdf.
25. National Forensic Laboratory Information System. NFLIS-Drug December 2020 Snapshot https://www.nflis.deadiversion.usdoj.gov/DesktopModules/ReportDownloads/Reports/N FLIS_Snapshot_122020.pdf.
26. Peterson, F. C. et al. Structural basis for selective activation of ABA receptors. Nat. Struct. Mol. Biol. 17, 1109-1113 (2010).
27. Vaidya, A. S. et al. Dynamic control of plant water use using designed ABA receptor agonists. Science 366, (2019)
28. Müller, K. M. et al. Nucleotide exchange and excision technology (NEXT) DNA shuffling: a robust method for DNA fragmentation and directed evolution. Nucleic Acids Res. 33, e117 (2005).
29. Okamoto, M. et al. Activation of dimeric ABA receptors elicits guard cell closure, ABA-regulated gene expression, and drought tolerance. Proc. Natl. Acad. Sci. U.S.A 110, 12132-12137 (2013).
30. Santiago, J. et al. Modulation of drought resistance by the abscisic acid receptor PYL5 through inhibition of clade A PP2Cs. Plant J. 60, 575-588 (2009).
31. Elzinga, D. et al. Defining and exploiting hypersensitivity hotspots to facilitate abscisic acid receptor agonist optimization. ACS Chem. Biol. (2019) doi: 10.1021/acschembio.8b00955.
32. McIsaac, R. S. et al. Synthetic gene expression perturbation systems with rapid, tunable, single-gene specificity in yeast. Nucleic Acids Res. 41, e57 (2013).
33. Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proc. Natl. Acad. Sci. U.S.A. 95, 14628-14633 (1998).
34. Sridharamurthy, M. et al. H2O2 inhibits ABA-signaling protein phosphatase HAB1. PLOS One 9, e113643 (2014).
35. Meinhard, M., Rodriguez, P. L. & Grill, E. The sensitivity of ABI2 to hydrogen peroxide links the abscisic acid-response regulator to redox signalling. Planta 214, 775-782 (2002).
36. Goldenzweig, A. et al. Automated Structure- and Sequence-Based Design of Proteins for High Bacterial Expression and Stability. Mol. Cell 63, 337-346 (2016).
37. Wrenbeck, E. E. et al. An Automated Data-Driven Pipeline for Improving Heterologous Enzyme Expression. ACS Synth. Biol. 8, 474-481 (2019).
38. Kabsch, W. XDS. Acta Crystallographica Section D Biological Crystallography vol. 66 125-132 (2010).
39. Adams, P. D. et al. PHENIX: a comprehensive Python-based system for macromolecular structure solution. Acta Crystallogr. D Biol. Crystallogr. 66, 213-221 (2010).
40. Schüttelkopf, A. W. & van Aalten, D. M. F. PRODRG: a tool for high-throughput crystallography of protein-ligand complexes. Acta Crystallogr. D Biol. Crystallogr. 60, 1355-1363 (2004).

### REFERENCE CITED BY AUTHOR AND YEAR

Adams, Paul D., Pavel V. Afonine, Gábor Bunkóczi, Vincent B. Chen, Ian W. Davis, Nathaniel Echols, Jeffrey J. Headd, et al. 2010. “PHENIX: A Comprehensive Python-Based System for Macromolecular Structure Solution.” Acta Crystallographica. Section D, Biological Crystallography 66 (Pt 2): 213-21.
Davis, Ian W., Andrew Leaver-Fay, Vincent B. Chen, Jeremy N. Block, Gary J. Kapral, Xueyi Wang, Laura W. Murray, et al. 2007. “MolProbity: All-Atom Contacts and Structure Validation for Proteins and Nucleic Acids.” Nucleic Acids Research 35 (Web Server issue): W375-83.
Dupeux, Florine, Julia Santiago, Katja Betz, Jamie Twycross, Sang-Youl Park, Lesia Rodriguez, Miguel Gonzalez-Guzman, et al. 2011. “A Thermodynamic Switch Modulates Abscisic Acid Receptor Sensitivity.” The EMBO Journal 30 (20): 4171-84.
Elzinga, Dezi, Erin Sternburg, Davide Sabbadin, Michael Bartsch, Sang-Youl Park, Aditya Vaidya, Assaf Mosquna, et al. 2019. “Defining and Exploiting Hypersensitivity Hotspots to Facilitate Abscisic Acid Receptor Agonist Optimization.” ACS Chemical Biology, January. https://doi.org/10.1021/acschembio.8b00955.
Emsley, Paul, and Kevin Cowtan. 2004. “Coot: Model-Building Tools for Molecular Graphics.” Acta Crystallographica. Section D, Biological Crystallography 60 (Pt 12 Pt 1): 2126-32.
Kabsch, Wolfgang. 2010. “XDS.” Acta Crystallographica. Section D, Biological Crystallography 66 (Pt 2): 125-32.
Kowalsky, Caitlin A., and Timothy A. Whitehead. 2016. “Determination of Binding Affinity upon Mutation for Type I Dockerin-Cobesin Complexes from Clostridium Thermocellum and Clostridium Cellulolyticum Using Deep Sequencing.” Proteins 84 (12): 1914-28.
Lin, Hsin-Ying, Sey-En Lin, Su-Fang Chien, and Ming-Kai Chern. 2011. “Electroporation for Three Commonly Used Yeast Strains for Two-Hybrid Screening Experiments.” Analytical Biochemistry 416 (1): 117-19.
Medina-Cucurella, Angélica V., Paul J. Steiner, Matthew S. Faber, Jesús Beltrán, Alexandra N. Borelli, Monica B. Kirby, Sean R. Cutler, and Timothy A. Whitehead. 2019. “User-Defined Single Pot Mutagenesis Using Unamplified Oligo Pools.” Protein Engineering, Design & Selection: PEDS 32 (1): 41-45.
Melcher, Karsten, Ley-Moy Ng, X. Edward Zhou, Fen-Fen Soon, Yong Xu, Kelly M. Suino-Powell, Sang-Youl Park, et al. 2009. “A Gate-Latch-Lock Mechanism for Hormone Signalling by Abscisic Acid Receptors.” Nature 462 (7273): 602-8.
Mosquna, Assaf, Francis C. Peterson, Sang-Youl Park, Jorge Lozano-Juste, Brian F Volkman, and Sean R. Cutler. 2011. “Potent and Selective Activation of Abscisic Acid Receptors in Vivo by Mutational Stabilization of Their Agonist-Bound Conformation.” Proceedings of the National Academy of Sciences of the United States of America 108 (51): 20838-43.
Müller, Kristian M., Sabine C. Stebel, Susanne Knall, Gregor Zipf, Hubert S. Bernauer, and Katja M. Arndt. 2005. “Nucleotide Exchange and Excision Technology (NEXT) DNA Shuffling: A Robust Method for DNA Fragmentation and Directed Evolution.” Nucleic Acids Research 33 (13): e117.
Mumberg, D., R. Müller, and M. Funk. 1995. “Yeast Vectors for the Controlled Expression of Heterologous Proteins in Different Genetic Backgrounds.” Gene 156 (1): 119-22.
Park, Sang-Youl, Francis C. Peterson, Assaf Mosquna, Jin Yao, Brian F. Volkman, and Sean R. Cutler. 2015. “Agrochemical Control of Plant Water Use Using Engineered Abscisic Acid Receptors.” Nature 520 (7548): 545-48.
Schüttelkopf, Alexander W., and Daan M. F. van Aalten. 2004. “PRODRG: A Tool for High-Throughput Crystallography of Protein-ligand Complexes.” Acta Crystallographica Section D Biological Crystallography. https://doi.org/10.1107/s0907444904011679.
Steiner, Paul J., Zachary T. Baumer, and Timothy A. Whitehead. 2020. “A Method for User-Defined Mutagenesis by Integrating Oligo Pool Synthesis Technology with Nicking Mutagenesis.” Bio-Protocol 10 (15): e3697.
Steiner, Paul J., Matthew A. Bedewitz, Angélica V. Medina-Cucurella, Sean R. Cutler, and Timothy A. Whitehead. 2020. “A Yeast Surface Display Platform for Plant Hormone Receptors: Toward Directed Evolution of New Biosensors.” AIChE Journal. American Institute of Chemical Engineers 66 (3). https://doi.org/10.1002/aic. 16767.
Vaidya, Aditya S., Jonathan D. M. Helander, Francis C. Peterson, Dezi Elzinga, Wim Dejonghe, Amita Kaundal, Sang-Youl Park, et al. 2019. “Dynamic Control of Plant Water Use Using Designed ABA Receptor Agonists.” Science 366 (6464). https://doi.org/10.1126/science.aaw8848.
Wrenbeck, Emily E., Justin R. Klesmith, James A. Stapleton, Adebola Adeniran, Keith E. J. Tyo, and Timothy A. Whitehead. 2016. “Plasmid-Based One-Pot Saturation Mutagenesis.” Nature Methods 13 (11): 928-30.

ILLUSTRATIVE SEQUENCES
(PYR1) polypeptide sequence binding pocket residues:
K59, I62, V81, V83, L87, A89, S92, E94, F108, I110, L117,
Y120, S122, E141, F159, A160, V163, V164, and N167
SEQ ID NO: 1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMFADTVVKLNLQKLAT
VAEAMARNSGDGSGSQVT
(PYL2) polypeptide sequence binding pocket residues:
K64, V67, V85, V87, L91, A93, S96, E98, F112, V114, L121,
Y124, S126, E147, F166, V167, V170, V171, and N174
SEQ ID NO: 2
MSSSPAVKGLTDEEQKTLEPVIKTYHQFEPDPTTCTSLITQRIHAPASVVWPLIRRFDN
PERYKHFVKRCRLISGDGDVGSVREVTVISGLPASTSTERLEFVDDDHRVLSFRVVGG
EHRLKNYKSVTSVNEFLNQDSGKVYTVVLESYTVDIPEGNTEEDTKMFVDTVVKLN
LQKLGVAATSAPMHDDE
ΔN HAB1^{T+} highlighted residues are mutated relative to
widltype HAB1 sequence; mutations occur at positions
393, 406, 411, 416, 455, 467, 481, 494, 499, 549, 565,
584, 629, 646, and 698 of SEQ ID NO: 5
SEQ ID NO: 3
GRSVYELDSIPLWGTVSIQGNASEMEAAFAVVPHFLKLPIKMLMGDHEGMSPSLTHL
TGHFFGVYDGHGGSKVADYCRDRLHEALAEEIERIKDELSKRNTGEGRQVQWKKVF
TNCFLTVDGEIEGKIGRAVVGSSDKVLEAVASETVGSTAVVALVCSSHIVVANCGDS
RAVLFRGKEAIPLSVDHKPDREDEYARIEAAGGKVIQWQGARVFGVLAMSRSIGDR
YLKPYVIPEPEVTFMPRSEEDECLILASDGLWDVMSNQEVCEIARRRILMWHKKNGA
PPLAERGKGIDPACQAAADYLSKLALQKGSKDNISIIVIDLKAQRKFKTRT*
ΔN-MBP-HAB1^{T+} Maltose binding protein HAB1^{T+} fusion protein;
MPB underlined
SEQ ID NO: 4
MNSSHHHHHHSSGMKIEEGKLVIWINGDKGYNGLAEVGKKFEKDTGIKVTVEHPDK
LEEKFPQVAATGDGPDIIFWAHDRFGGYAQSGLLAEITPDKAFQDKLYPFTWDAVRY
NGKLIAYPIAVEALSLIYNKDLLPNPPKTWEEIPALDKELKAKGKSALMFNLQEPYFT
WPLIAADGGYAFKYENGKYDIKDVGVDNAGAKAGLTFLVDLIKNKHMNADTDYSI
AEAAFNKGETAMTINGPWAWSNIDTSKVNYGVTVLPTFKGQPSKPFVGVLSAGINA
ASPNKELAKEFLENYLLTDEGLEAVNKDKPLGAVALKSYEEELVKDPRIAATMENA
QKGEIMPNIPQMSAFWYAVRTAVINAASGRQTVDEALKDAQTGGGGGRSVYELDSI
PLWGTVSIQGNASEMEAAFAVVPHFLKLPIKMLMGDHEGMSPSLTHLTGHFFGVYD
GHGGSKVADYCRDRLHEALAEEIERIKDELSKRNTGEGRQVQWKKVFTNCFLTVDG
EIEGKIGRAVVGSSDKVLEAVASETVGSTAVVALVCSSHIVVANCGDSRAVLFRGKE
AIPLSVDHKPDREDEYARIEAAGGKVIQWQGARVFGVLAMSRSIGDRYLKPYVIPEP
EVTFMPRSEEDECLILASDGLWDVMSNQEVCEIARRRILMWHKKNGAPPLAERGKGI
DPACQAAADYLSKLALQKGSKDNISIIVIDLKAQRKFKTRT*
HAB1 full-length wildtype amino acid sequence; N-terminal
177 residues are deleted in ΔN-HAB1
SEQ ID NO: 5
MEEMTPAVAM TLSLAANTMC ESSPVEITQL KNVTDAADLL SDSENQSFCN
GGTECTMEDV SELEEVGEQD LLKTLSDTRS GSSNVFDEDD VLSVVEDNSA
VISEGLLVVD AGSELSLSNT AMEIDNGRVL ATAIIVGESS IEQVPTAEVL
IAGVNQDTNT SEVVIRLPDE NSNHLVKGRS VYELDCIPLW GTVSIQGNRS
EMEDAFAVSP HFLKLPIKML MGDHEGMSPS LTHLTGHFFG VYDGHGGHKV
ADYCRDRLHF ALAEEIERIK DELCKRNTGE GRQVQWDKVF TSCFLTVDGE
IEGKIGRAVV GSSDKVLEAV ASETVGSTAV VALVCSSHIV VSNCGDSRAV
LFRGKEAMPL SVDHKPDRED EYARIENAGG KVIQWQGARV FGVLAMSRSI
GDRYLKPYVI PEPEVTFMPR SREDECLILA SDGLWDVMNN QEVCEIARRR
ILMWHKKNGA PPLAERGKGI DPACQAAADY LSMLALQKGS KDNISIIVID
LKAQRKFKTR T
CANNIBINOID RECEPTOR SEQUENCES-mutated residues relative
to SEQ ID NO: 1 are highlighted
(SEQ ID NOS: 6-22)
>PYR1-JWH-007
MPSELTPEERSELKNSIAEFHTYQLDPGSCSSLHAQRIHAPPELVWSIVRRFDKPQTYK
HFIKSCSVEQNFEMRVGCTRDVIWISGLPANTSTERLDILDDERRVTGFSIIGGEHRLT
NGKSVTTVHRFEKENRIWTVVLESYVVDMPEGNSEDDTRMAIDTVVKLNLQKLATV
AEAMARNSGDGSGSQVTVDSRAL*
>PYR1-JWH-015
MPSELTPEERSELKNSIAEFHTYQLDPGSCSSLHAQRIHAPPELVWSIVRRFDKPQTYN
HFIKSCSVEQNFEMRVGCTRDVIVISGLPANTSTERLDILDDERRVTGFSIIGGEHRLT
NHKSVTTVHRFEKENRIWTVVLESYVVDMPEGNSEDDTRMLGDTVVKLNLQKLAT
VAEAMARNSGDGSGSQVTVDSRAL*
>PYR1-JWH-016
MPSELTPEERSELKNSIAEFHTYQLDPGSCSSLHAQRIHAPPELVWSIVRRFDKPQTYK
HFIKSCSVEQNFEMRVGCTRDIIVISGLPANTSTERLDILDDERRVTGFSIIGGEHRLTN
GKSVTTVHRFEKENRIWTVVLESYVVDMPEGNSEDDTRMLGDTVVKLNLQKLATV
AEAMARNSGDGSGSQVTVDSRAL*
>PYR1-JWH-018
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NGKSVTTVHRFEKENRIWTVVLESYVVDMPEGNSEDDTRIFGDTVVKLNLQKLATV
AEAMARNSGDGSGSQVTVDSRAL*
>PYR1-JWH-072
MPSGLTPEERSELKNSIAEFHTYQLDPGSCSSLHAQRIHAPPELVWSIVRRFDKPQTYN
HFIKSCSVEQNFEMRVGCTRDVIVISGLPANTSTERLDILDDERRVTGFSIIGGEHRLT
NHKSVTTVHRFEKENRIWTVVLESYVVDMPEGNSEDDTRMLGDTVVKLNLQKLAT
VAEAMARNSGDGSGSQVTVDSRAL*
>PYR1-JWH-167
MPSELTPEERSELKNSIAEFHTYQLDPGSCSSLHAQRIHAPPELVWSIVRRFDKPQTFK
HFIKSCSVEQNFEMRVGCTRDMIFISGLPANTSTERLDILDDERRVTGFSIIGGEQRLT
NGKSVTTVHRFEKENRIWTVVLESYVVDMPEGNSEDDTRMLGDTVVKLNLQKLAT
VAEAMARNSGGGSGSQVTVDSRAL*
>PYR1-JWH-193
MPSELTPEERSELKNSIAEFHTYQLDPGSCSSLHAQRIHAPPELVWSIVRRFDKPQTYK
HFIKSCSVEQNFEMRVGCTRDVIVISGMPVNTSTERLDILDDKRRVTGFSIIGGEHRLT
NGKSVTTVHRFEKENRIWTVVLESYVVDMPEGNSEDDTRMVGDTVVKLNLQKLAT
VAEAMARNSGDGSGSQVTVDSRAL*
>PYR1-WIN
MPSGLTPEERSELKNSIAEFHTHQLGPGSCSSLHAQRIHAPPELVWSIVRRFDKPQTYQ
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMAIDTVVKLNLQKLATV
AEAMARNSGDGSGSQVTVDSRAL*
>PYR1-4FMDMB
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEQRLT
NGKSVTTVHRFEKENRIWTVVLESYVVDMPEGNSEDDTRMVGDTVVKLNLQKLAT
VAEAMARNSGDGSGSQVTVDSRAL*
>PYR1-AB-PINACA
MPSGLTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEQRLT
NGKSVTTVHRFEKENRIWTVVLESYVVDMPEGNSEDDTRMVGDTVVKLNLQKLAT
VAEAMARNSGDGSGSQVTVDSRAL*
>PYR1-CP
MPSELTPEERSELKNSIAEFHTYQLDPGSCSSLHAQRIHAPPELVWSIVRRFDKPQTYA
HFIKSCSVEQNFEMRVGCTRDVILISGLPANTSTERLDILDDERRVTGFSIIGGEHRLTN
YKSVTTVHRFEKENRIWTVVLFSYVVDMPEGNSEDDTRMFADTVVKLNLQKLATV
AEAMARNSGDGSGSQVTVDSRAL*
>PYR1-CBDA
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEQRLT
NGKSVTTVHRFEKENRIWTVVLESYVVDMPEGNSEDDTRMFADTVWKLNLQKLAT
VAEAMARNSGDGSGSQATVDSRAL*
>PYR1-CBDA-high-affinity
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEQRLT
NGKSVTTVHRFEKENRIWTVVLMSYVVDMPEGNSEDDTRMFADTVWKLNLQKLAT
VAEAMARNSGDGSGSQVTVDSRAL*
>PYR1-Δ9-THC
MPSGLTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEQRLT
NGKSVTTVHRFEKENRIWTVVLESYVVDMPEGNSEDDTRMFGDTVVKLNLQKLAT
VAEAMARNSGDGSGSQVTVDSRAL*
>PYR1-CBGA
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEQRLT
NGKSVTTVHRFEKENRIWTVVLESYVVDMPEGNSEDDTRMLGDTVVKLNLQKLAT
VAEAMARNSGDGSGSQVTVDSRAL*
>PYR1-CBGVA
MPSGLTPEERSELKNSIAEFHTYQLDPGSCSSLHAQRIRAPPELVWSIVRRFDKPQTYR
HLIKSCSVEQNFEMRVGCTRDVIVISGLPANTSTERLDILDDERRVTGFSVIGGDQRLT
NGKSVTTVHRFEKENRIWTVVLMSYVVDMPEGNSEDDTRMFADTVVKLNLQKLAT
VAEAMARNSGDGSGSQVTVDSRAL*
>PYR1-CBDVA
MPSGLTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEQRLT
NGKSVTTVHRFEKENRIWTVVLMSYVVDMPEGNSEDDTRMFADTVWKLNLQKLAT
VAEAMARNSGDGSGSQVTVDSRAL*
ORGANOPHOSPHATE RECEPTOR SEQUENCES-mutated residue residues
relative to SEQ ID NO: 1 are highlighted
SEQ ID NOS: 23-30
>PYR1-DIAZI
MPSELTPGERSELKNSIAEFHTYQLDPGSCSSLHAQRIHAPPELVWSIVRRFDKPQTYK
HFIKSCSVEQNFEMRVGCTRDYIVISGMPANTSTERLDILDDERRVTGYSIIGGEHRLT
NYKSVTTVHRFEKENRIWTVVLESYVVDMPEGNSEDDTRVGVDTVVKLNLQKLAT
VAEAMARNSGDGSGSQVTVDSRAL*
>PYR1-PIRI
MPSELTPEERSELKNSIAEFHTYQLDPGSCSSLHAQRIHAPPELVWSIVRRFDKPQTYR
HFIKSCSVEQNFEMRVGCTRDVIVISGLPANTMTERLDILDDERRVTGFSIIGGEHRLT
SYKQVTTVHRFGKENRIWTVVLESYVVDMPEGNSEDDTRMTTDTVVKLNLQKLAT
AAEAMARNSGDGSGSQVTVDSRAL*
>PYR1-AZIN
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMFADTWVKLNLQKLAT
VAEAMARNSGDGSGSQVTVDSRAL*
>PYR1-PARATHION
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NLKNVTTVHRFEKENRIWTVVLESYVVDMPEGNSEDDTRMFADTVVKLNLQKLAT
VAEAMARNSGDGSGSQVTVDSRAL*
>PYR1-DISULFOTON
MPSELTPEERSELKNSIAEFHTYQLDPGSCSSLHAQRIHAPPELVWSIVRRFDKPQTYN
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMLADTVVKLNLQKLAT
VAEAMARNSGDGSGSQVTVDSRAL*
>PYR1-CHLORFENVINPHOS
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGLSIIGGEHRLT
NYKSVTTVHRFEKENRIWTVVLESYVVDMPEGNSEDDTRMLADTVVKLNLQKLAT
VAEAMARNSGDGSGSQVTVDSRAL*
>PYR1-DIMETHOATE
MPSELTPEERSELKNSIAEFHTYQLDPGSCSSLHAQRIHAPPELVWSIVRRFDKPQTYK
HFIKSCSVEQNFEMRVGCTRDLIVISGLPANTSTERLDILDDERRVTGFSIIGGEHRLTN
YKSVTTVHRFEKENRIWTVVLESYVVDMPEGNSEDDTRMFADTVVKLGLQKLATV
AEAMARNSGDGSGSQVTVDSRAL*
>PYR1-TBP-new
MPSELTPEERSELKNSIAEFHTYQLDPGSCSSLHAQRIHAPPELVWSIVRRFDKPQTYR
HFIKSCSVEQNFEMRAGCTRDVILISGLPANTSTERLDILDDERRVTGYSIIGGEHRLT
NYKSVTTVHRFEKENRIWTVVLESYVVDMLEGNSEDDTRMFADTVHKLNLRKLAT
VAEAMARNSGDGSGSQVTVDSRAL*
LOWER AFFINITY CANNABINOID RECEPTOR SEQUENCES-mutated
residue are highlighted
(SEQ ID NOS 120-138)
>PYR1-JWH-193-AB-PINACA
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NGKSVTTVHRFEKENRIWTVVLESYVVDMPEGNSEDDTRMVGDTVVKLNLQKLAT
VAEAMARNSGDGSGSQVTVDSRAL*
>PYR1-WIN-C2
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMSLDTVVKLNLQKLAT
VAEAMARNSGDGSGSQVTVDSRAL*
>PYR1-WIN-C4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMSVDTVVKLNLQKLAT
VAEAMARNSGDGSGSQVTVDSRAL*
>PYR1-WIN-C5
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMTLDTVVKLNLQKLAT
VAEAMARNSGDGSGSQVTVDSRAL*
>PYR1-WIN-C6
MPSELTPEERSELKNSIAEFHTYQLDPGSCSSLHAQRIHAPPELVWSIVRRFDKPQTYQ
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMAIDTVVKLNLQKLATV
AEAMARNSGDGSGSQVTVDSRAL*
>PYR1-WIN-C8
MPSELTPEERSELKNSIAEFHTYQLDPGSCSSLHAQRIHAPPELVWSIVRRFDKPQTYQ
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMAIDTVVKLNLRKLATV
AEAMARNSGDGSGSQVTVDSRAL*
>PYR1-WIN-C9
MPSELTPEERSELKNSIAEFHTHQLGPGSCSSLHAQRIHAPPELVWSIVRRFDKPQTYQ
HFIKSCSVEQNFEMRVGCTRDVIVISGLPANTSTERLDILDDERRVTGFSIGGEHRLT
NYKSVTTVHRFEKENRIWTVVLESYVVDMPEGNSEDDTRMAIDTVVKLNLQKLATV
AEAMARNSGDGSGSQVTVDSRAL*
>PYR1-JWH-015-C1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMAADTVHKLNLQKLAT
VAEAMARNSGDGSGSQVTVDSRAL*
>PYR1-JWH-015-JWH-072-JWH-018-JWH-030
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NGKSVTTVHRFEKENRIWTVVLESYVVDMPEGNSEDDTRMFGDTVVKLNLQKLAT
VAEAMARNSGDGSGSQVTVDSRAL*
>PYR1-JWH-015-JWH-072-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NHKSVTTVHRFEKENRIWTVVLESYVVDMPEGNSEDDTRMFGDTVVKLNLQKLAT
VAEAMARNSGDGSGSQVTVDSRAL*
>PYR1-JWH-015-JWH-072-JWH-016-JWH-030
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMFGDTVVKLNLQKLAT
VAEAMARNSGDGSGSQVTVDSRAL*
>PYR1-JWH-015-C4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMAIDTVVKLNLQKLATV
AEAMARNSGDGSGSQVTVDSRAL*
>PYR1-JWH-015-JWH-072-2
MPSELTPEERSELKNSIAEFHTYQLDPGSCSSLHAQRIHAPPELVWSIVRRFDKPQTYN
HFIKSCSVEQNFEMRVGCTRDVIVISGLPANTSTERLDILDDERRVTGFSIIGGEHRLT
NHKSVTTVHRFEKENRIWTVVLESYVVDMPEGNSEDDTRMFGDTVVKLNLQKLAT
VAEAMARNSGDGSGSQVTVDSRAL*
>PYR1-JWH-072-C3
MPSELTPEERSELKNSIAEFHTYQLDPGSCSSLHAQRIHAPPELVWSIVRRFDKPQTYT
HFIKSCSVEQNFEMRVGCTRDVIVISGLPANTSTERLDILDDERRVTGFSIIGGEHRLT
NGKSVTTVHRFEKENRIWTVVLESYVVDMPEGNSEDDTRMFGDTVVKLNLQKLAT
VAEAMARNSGDGSGSQVTVDSRAL*
>PYR1-JWH-007-C1
MPSELTPEERSELKNSIAEFHTYQLDPGSCSSLHAQRIHAPPELVWSIVRRFDKPQTYS
HFIKSCSVEQNFEMRVGCTRDVIWISGLPANTSTERLDILDDERRVTGFSIIGGEHRLT
NGKSVTTVHRFEKENRIWTVVLESYVVDMPEGNSEDDTRMAADTVVKLNLQKLAT
VAEAMARNSGDGSGSQVTVDSRAL*
>PYR1-JWH-016-C2
MPSELTPEERSELKNSIAEFHTYQLDPGSCSSLHAQRIHAPPELVWSIVRRFDKPQTYK
HFIKSCSVEQNFEMRVGCTRDIIVISGLPANTSTERLDILDDERRVTGFSIIGGEHRLTN
GKSVTTVHRFEKENRIWTVVLESYVVDMPEGNSEDDTRMFGDTVVKLNLQKLATV
AEAMARNSGDGSGSQVTVDSRAL*
>PYR1-JWH-167-C1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NGKSVTTVHRFEKENRIWTVVLESYVVDMPEGNSEDDTRMLGDTVVKLNLQKLAT
VAEAMARNSGDGSGSQVTVDSRAL*
>PYR1-JWH-167-C2
MPSELTPEERSELKNSIAEFHTYQLDPGSCSSLHAQRIHAPPELVWSIVRRFDKPQTYS
HFIKSCSVEQNFEMRVGCTRDVIWISGLPANTSTERLDILDDERRVTGFSIIGGEQRLT
NGKSVTTVHRFEKENRIWTVVLESYVVDMPEGNSEDDTRMLGDTVVKLNLQKLAT
VAEAMARNSGDGSGSQVTVDSRAL*
>PYR1-CBDA-1
MPSGLTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEQRLT
NGKSVTTVHRFEKENRIWTVVLMSYVVDMPEGNSEDDTRMFADTVVKLNLQKLAT
VAEAMARNSGDGSGSQVTVDSRAL*
LOWER AFFINITY ORGANOPHOSPHATE RECEPTOR SEQUENCES-mutated residues
are highlighted
(SEQ ID NOS 139-178, 27, and 179-183)
>PYR1-DIAZINON-C1
MPSELTPEERSELKNSIAEFHTYQLDPGSCSSLHAQRIHAPPELVWSIVRRFDKPQTYK
HFIKSCSVEQNFEMRVGCTRDVIVISGPPANTSTERLDILDDERRVTGFSIIGGEHRLTN
AKSVTTVHRFEKENRIWTVVLESYVVDMPEGNSEDDTRMFADTVVKLNLQKLATV
AEAMARNSGDGSGSQVTVDSRAL*
>PYR1-DIAZINON-C3
MPSELTPEERSELKNSIAEFHTYQLDPGSCSSLHAQRIHAPPELVWSIVRRFDKPQTYK
HFIKSCSVEQNFEMRVGCTRDVIVISGMPANTSTERLDILDDERRVTGFSIIGGEHRLT
NYKSVTTVHRFEKENRIWTVVLESYVVDMPEGNSEDDTRMGADTVVKLNLQKLAT
VAEAMARNSGDGSGSQVTVDSRAL*
>PYR1-DIAZINON-C4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGYSIIGGEHRLT
NYKSVTTVHRFEKENRIWTVVLESYVVDMPEGNSEDDTRMGADTVVKLNLQKLAT
VAEAMARNSGDGSGSQVTVDSRAL*
>PYR1-DIAZINON-C5
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMLIDTVVKLNLQKLATV
AEAMARNSGDGSGSQVTVDSRAL*
>PYR1-DIAZINON-C8
MPSELTPEERSELKNSIAEFHTYQLDPGSCSSLHAQRIHAPPELVWSIVRRFDKPQTY
MHFIKSCSVEQNFEMRVGCTRDVIMISGLPANTSTERLDILDDERRVTGFSIIGGEHRL
TNAKSVTTVHRFEKENRIWTVVLESYVVDMPEGNSEDDTRMFADTVVKLNLQKLA
TVAEAMARNSGDGSGSQVTVDSRAL*
>PYR1-DIAZINON-C9
MPSELTPEERSELKNSIAEFHTYQLDPGSCSSLHAQRIHAPPELVWSIVRRFDKPQTYK
HFIKSCSVEQNFEMRVGCTRDVIMISGLPANTSTERLDILDDERRVTGFSIIGGEHRLT
NAKSVTTVHRFEKENRIWTVVLESYVVDMPEGNSEDDTRMFADTVVKLNLQKLAT
VAEAMARNSGDGSGSQVTVDSRAL*
>PYR1-DIAZINON-C10
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGYSIIGGEHRLT
NAKSVTTVHRFEKENRIWTVVLESYVVDMPEGNSEDDTRMFADTVVKLNLQKLAT
VAEAMARNSGDGSGSQVTVDSRAL*
>PYR1-DIAZINON-11
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMFADTVVKLNLQKLAT
VAEAMARNSGDGSGSQVTVDSRAL*
>PYR1-DIAZINON-12
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGYSIIGGEHRLT
NYKSVTTVHRFEKENRIWTVVLESYVVDMPEGNSEGDTRMGVDTVVKLNLQKLAT
VAEAMARNSGDGSGSQVTVDSRAL*
>PYR1-DIAZINON-13
MPSELTPEERSELKNSIAEFHTYQLDPGSCSSLHAQRIHAPPELVWSIVRRFDKPQTYK
HFIKSCSVEQNFEMRVGCTRDVIVISGMPANTSTERLDILDDERRVTGYSIIGGEHRLT
NYKSVTTVHRFEKENRIWTVVLESYVVDMPEGNSEDDTRMGVDTVVKLNLQKLAT
VAEAMARNSGDGSGSQVTVDSRAL*
>PYR1-DIAZINON-14
MPSELTPEERSELKNSIAEFHTYQLDPGSCSSLHAQRIHAPPELVWSIVRRFDKPQTYK
HFIKSCSVEQNFEMRVGCTRDYIVISGMPANTSTERLDILDDERRVTGYSIIGGEHRLT
NYKSVTTVHRFEKENRIWTVVLESYVVDMPEGNSEDDTRMGVDTVVKLNLQKLAT
VAEAMARNSGDGSGSQVTVDSRAL*
>PYR1-PIRIMIPHOS-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMLADTVVKLNLQKLAT
VAEAMARNSGDGSGSQVTVDSRAL*
>PYR1-PIRIMIPHOS-DIAZI-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMTADTVVKLNLQKLAT
VAEAMARNSGDGSGSQVTVDSRAL*
>PYR1-PIRIMIPHOS-3
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMIADTVIKLNLQKLATV
AEAMARNSGDGSGSQVTVDSRAL*
>PYR1-PIRIMIPHOS-4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMTADTHVKLNLQKLAT
VAEAMARNSGDGSGSQVTVDSRAL*
>PYR1-PIRIMIPHOS-5
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMINDTVVKLNLQKLATV
AEAMARNSGDGSGSQVTVDSRAL*
>PYR1-PIRIMIPHOS-6
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NAKSVTTVHRFEKENRIWTVVLESYVVDMPEGNSEDDTRMAADTVVKLNLQKLAT
VAEAMARNSGDGSGSQVTVDSRAL*
>PYR1-PIRIMIPHOS-CLORFENVINPHOS-1
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMLADTVVKLNLQKLAT
VAEAMARNSGDGSGSQVTVDSRAL*
>PYR1-PIRIMIPHOS-8
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSAIGGEHRLT
NYKSVTTVHRFEKENRIWTVVLESYVVDMPEGNSEDDTRMIADTVVKLNLQKLATV
AEAMARNSGDGSGSQVTVDSRAL*
>PYR1-PIRIMIPHOS-10
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMTADTVVKLNLQKLAT
VAEAMARNSGDGSGSQVTVDSRAL*
>PYR1-PIRIMIPHOS-11
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMCADTVVKLNLQKLAT
VAEAMARNSGDGSGSQVTVDSRAL*
>PYR1-PIRIMIPHOS-12
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMIADTVVKLNLQKLATV
AEAMARNSGDGSGSQVTVDSRAL*
>PYR1-PIRIMIPHOS-13
MPSELTPEERSELKNPIAEFHTYQLDPGGCSSLHARRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMTADTVVKLNLQKLAT
VAEAMARNSGDGSGSQVTVDSRAL*
>PYR1-PIRIMIPHOS-14
MPSELTPEERSELKNSIAEFHTYQLDPGSCSSLHAQRIHAPPELVWSIVRRFDKPQTYR
HFIKSCSVEQNFEMRVGCTRDVIVISGLPANTMTERLDILDDERRVTGFSIIGGEHRLT
NYKSVTTVHRFEKENRIWTVVLESYVVDMPEGNSEDDTRMTADTVVKLNLQKLAT
AAEAMARNSGDGSGSQVTVDSRAL*
>PYR1-PIRIMIPHOS-15
MPSELTPEERSELKNSIAEFHTYQLDPGSCSSLHAQRIHAPPELVWSIVRRFDKPQTYR
HFIKSCSVEQNFEMRVGCTRDVIVISGLPANTMTERLDILDDERRVTGFSIIGGEHRLT
NYKQVTTVHRFGKENRIWTVVLESYVVDMPEGNSEDDTRMTADTVVKLNLQKLAT
AAEAMARNSGDGSGSQVTVDSRAL*
>PYR1-CHLORFENVINPHOS-2
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGASIIGGEHRLT
NYKSVTTVHRFEKENRIWTVVLESYVVDMPEGNSEDDTRMFADTVVKLNLQKLAT
VAEAMARNSGDGSGSQVTVDSRAL*
>PYR1-CHLORFENVINPHOS-3
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSAIGGEHRLT
NYKSVTTVHRFEKENRIWTVVLESYVVDMPEGNSEDDTRMGADTVVKLNLQKLAT
VAEAMARNSGDGSGSQVTVDSRAL*
>PYR1-CHLORFENVINPHOS-4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGLSIIGGEHRLT
NYKSVTTVHRFEKENRIWTVVLESYVVDMPEGNSEDDTRMLADTVVKLNLQKLAT
VAEAMARNSGDGSGSQVTVDSRAL*
>PYR1-CHLORFENVINPHOS-5
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSAIGGEHRLT
NYKSVTTVHRFEKENRIWTVVLESYVVDMPEGNSEDDTRMLADTVVKLILQKLATV
AEAMARNSGDGSGSQVTVDSRAL*
>PYR1-CHLORFENVINPHOS-6
MPSELTPEERSELKNSIAEFHTYQLDPGSCSSLHAQRIHAPPELVWSIVRRFDKPQTYR
HFIKSCSVEQNFEMRVGCTRDVIIISGLPANTSTERLDILDDERRVTGFSIIGGEHRLTN
YKSVTTVHRFEKENRIWTVVLESYVVDMPEGNSEDDTRMLADTVVKLNLQKLATV
AEAMARNSGDGSGSQVTVDSRAL*
>PYR1-CHLORFENVINPHOS-7
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRRWTVVLESYVVDMPEGNSEDDTRMFADWVVKLNLQKLA
TVAEAMARNSGDGSGSQVTVDSRAL*
>PYR1-DIMETHOATE-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMFADTVVKLGLQKLAT
VAEAMARNSGDGSGSQVTVDSRAL*
>PYR1-DIMETHOATE-2
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRTFADTVVKLNLQKLATV
AEAMARNSGDGSGSQVTVDSRAL*
>PYR1-DIMETHOATE-3
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMHADTVVKLGLQKLAT
VAEAMARNSGDGSGSQVTVDSRAL*
>PYR1-DIMETHOATE-4
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKGVTTVHRFEKENRIWTVVLESYVVDMPEGNSEDDTRMFADTVVKLGLQKLAT
VAEAMARNSGDGSGSQVTVDSRAL*
>PYR1-DIMETHOATE-5
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGWSIIGGEHRLT
NYKSVTTVHRFEKENRIWTVVLESYVVDMPEGNSEDDTRMFADTVVKLGLQKLAT
VAEAMARNSGDGSGSQVTVDSRAL*
>PYR1-DIMETHOATE-7
MPSELTPEERSELKNSIAEFHTYQLDPGSCSSLHAQRIHAPPELVWSIVRRFDKPQTYK
HFIKSCSVEQNFEMRVGCTRDVIVISGIPANTSTERLDILDDERRVTGFSIIGGEHRLTN
YKSVTTVHRFEKENRIWTVVLESYVVDMPEGNSEDDTRMFADTVVKLGLQKLATV
AEAMARNSGDGSGSQVTVDSRAL*
>PYR1-DIMETHOATE-8
MPSELTPEERSELKNSIAEFHTYQLDPGSCSSLHAQRIHAPPELVWSIVRRFDKPQTYR
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRVFADTVVKLNLQKLATV
AEAMARNSGDGSGSQVTVDSRAL*
>PYR1-DISULFOTON-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMALDTVVKLNLQKLAT
VAEAMARNSGDGSGSQVTVDSRAL*
>PYR1-DISULFOTON-2
MPSELTPEERSELKNSIAEFHTYQLDPGSCSSLHAQRIHAPPELVWSIVRRFDKPQTYK
HFIKSCSVEQNFEMRVGCTRDVIVISGMPANTSTERLDILDDERRVTGFSIIGGEHRLT
NHKSVTTVHRFEKENRIWTVVLESYVVDMPEGNSEDDTRMFADTVVKLNLQKLAT
VAEAMARNSGDGSGSQVTVDSRAL*
>PYR1-DISULFOTON-3
MPSELTPEERSELKNSIAEFHTYQLDPGSCSSLHAQRIHAPPELVWSIVRRFDKPQTYN
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMLADTVVKLNLQKLAT
VAEAMARNSGDGSGSQVTVDSRAL*
>PYR1-TBP-1
MPSELTPEERSELKNSIAEFHTYQLDPGSCSSLHAQRIHAPPELVWSIVRRFDKPQTYK
HFIKSCSVEQNFEMRVGCTRDVIVISGLPANTSTDRLDILDDERRVTGFSIIGGEHRLT
NYKSVTTVHRFEKENRIWTVVLESYVVDMPEGNSEDDTRMFADTVHKLNLQKLAT
VAEAMARNSGDGSGSQVTVDSRAL*
>PYR1-TBP-2
MPSELTPEERSELKNSIAEFHTYQLDPGSCSSLHAQRIHAPPELVWSIVRRFDKPQTYK
HFIKSCSVEQNFEMRVGCTRDVIAISGLPANTSTDRLDILDDERRVTGFSIIGGEHRLT
NYKSVTTVHRFEKENRIWTVVLESYVVDMPEGNSEDDTRMFADTVHKLNLQKLAT
VAEAMARNSGGGSGSQVTVDSRAL*
>PYR1-TBP-3
MPSELTPEERSELKNSIAEFHTYQLDPGSCSSLHAQRIHAPPELVWSIVRRFDKPQTYR
HFIKSCSVEQNFEMRAGCTRDVILISGLPANTSTERLDILDDERRVTGFSIIGGEHRLTN
YKSVTTVHRFEKENRIWTVVLESYVVDMLEGNSEDDTRMFADTVVHLNLQKLATV
AEAMARNSGDGSGSQVTVDSRAL*
>PYR1-TBP-4
MPSELTPEERSELKNSIAEFHTYQLDPGSCSSLHAQRIHAPPELVWSIVRRFDKPQTYR
HFIKSCSVEQNFEMRAGCTRDVILISGLPANTSTERLDILDDERRVTGFSIIGGEHRLIN
YKSVTTVHRFEKENRIWTVVLESYVVDMLEGNSEDDTRMFADTVVHLNLQKLATV
AEAMARNSGGGSGSQVTVDSRAL*
>PYR1-TBP-5
MPSELAPEERSELKNSIAEFHTYQLDPGSCSSLHAQRIHAPPELVWSIVRRFDKPQTYR
HFIKSCSVEQNFEMRAGCTRDVILISGLPANTSTERLDILDDERRVTGYSIIGGEHRLT
NYKSVTTVHRFEKENRIWTVVLESYVVDMLEGNSEDDTRMFADTVVHLNLQKLAT
VAEAMARNSGDGSGSQVTVDSRAL*
CANNABINOID AND ORGANOPHOSPHATE RECEPTOR SEQUENCES-mutated
residues are highlighted
PYR1-WIN-DIAZI-1
SEQ ID NO: 31
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMGVDTVVKLNLQKLAT
VAEAMARNSGDGSGSQVTVDSRAL*
>PYR1-WIN-DIAZI-2
SEQ ID NO: 32
MPSELTPEERSELKNSIAEFHTYQLDPGSCSSLHAQRIHAPPELVWSIVRRFDKPQTYK
HFIKSCSVEQNFEMRVGCTRDVIVISGLPANTSTERLDILDDERRVTGFSIIGGEHRLT
NYKSVTTVHRFEKENRIWTVVLESYVVDMPEGNSEDDTRMGIDTVVKLNLQKLATV
AEAMARNSGDGSGSQVTVDSRAL*
PYL2 sensor sequences:
PYL2^{T}
SEQ ID NO: 33
MSSSPAVKGLTDEEQKTLEPVIKTYHQFEPDPTTCTSLITQRIHAPASVVWPLIRRFDN
PERYRHFIKRCRLISGDGDAGSVREVTLISGLPASTSTERLEFVDDDHRVLSYRIVGG
EHRLKNYKSVTSVNEFLNQDSGKVYTVVLESYTVDILEGNTEEDTKMFADTVHKLN
LRKLGVAATSAPMHDDE*
PYL2^{DIAZI}
SEQ ID NO: 34
MSSSPAVKGLTDEEQKTLEPVIKTYHQFEPDPTTCTSLITQRIHAPASVVWPLIRRFDN
PERYKHFVKRCRLISGDGDVGSVREYTVISGMPASTSTERLEFVDDDHRVLSYRVVG
GEHRLKNYKSVTSVNEFLNQDSGKVYTVVLESYTVDIPEGNTEEDTKVGVDTVVKL
NLQKLGVAATSAPMHDDE*
PYL2^{CBDA}
SEQ ID NO: 35
MSSSPAVKGLTDEEQKTLEPVIKTYHQFEPDPTTCTSLITQRIHAPASVVWPLIRRFDN
PERYRHFVKRCRLISGDGDVGSVREVTVISGLPASTSTERLEFVDDDHRVLSFRVVGG
EQRLKNGKSVTSVNEFLNQDSGKVYTVVLMSYTVDIPEGNTEEDTKMFVDTVWKL
NLQKLGVAATSAPMHDDE*
PYL2^{4F}
SEQ ID NO: 36
MSSSPAVKGLTDEEQKTLEPVIKTYHQFEPDPTTCTSLITQRIHAPASVVWPLIRRFDN
PERYKHFVKRCRLISGDGDVGSVREVTVISGLPASTSTERLEFVDDDHRVLSFRVVGG
EQRLKNGKSVTSVNEFLNQDSGKVYTVVLESYTVDIPEGNTEEDTKMVGDTVVKLN
LQKLGVAATSAPMHDDE*
PYL2^{WIN}
SEQ ID NO: 37
MSSSPAVKGLTDEEQKTLEPVIKTYHQFEPDPTTCTSLITQRIHAPASVVWPLIRRFDN
PERYQHFVKRCRLISGDGDVGSVREVTVISGLPASTSTERLEFVDDDHRVLSFRVVG
GEHRLKNYKSVTSVNEFLNQDSGKVYTVVLESYTVDIPEGNTEEDTKMAIDTVVKL
NLQKLGVAATSAPMHDDE*

1. A library comprising a plurality of polypeptides, wherein each polypeptide contained in the plurality of polypeptides comprises a mutated PYR/PYL receptor polypeptide comprising I, L, R, V, or Y at position 62, relative to the corresponding position of SEQ ID NO:1; and at least two amino acid substitutions, relative to the corresponding position of SEQ ID NO: 1, selected from positions 59, 83, 89, 92, 94, 108, 117, 120, 122, 141, 159, 160, and 163, wherein the at least two amino acid substitutions differ among members of the library and are independently selected from A, D, E, F, G, H, I, K, L, M, N, Q, R, S, T, V, W and Y.

2. The library of claim 1, wherein each polypeptide further comprises at least one of the following: I, L, M, R, T, V, or Y at position 81; A, F, G, I, L, M, V, W, or Y at position 87; or A, F, I, K, L, M R, T, V, or W at position 110; or I, L, M, R, T, V, or Y at position 81; A, F, G, I, L, M, V, W, or Y at position 87; and A, F, I, K, L, M R, T, V, and W at position 110.

3. (canceled)

4. The library of claim 1, wherein at least one, at least two, at least three, or all four of positions 62, 81, 87, and 110 of each polypeptide are substituted relative to the corresponding position of SEQ ID NO:1.

5. The library of claim 1, wherein the plurality of polypeptides comprises polypeptides comprising at least three amino acid substitutions, relative to SEQ ID NO:1, selected from positions 59, 83, 89, 92, 94, 108, 117, 120, 122, 141, 159, 160, and 163; and wherein the substitutions are independently selected from A, D, E, F, G, H, I, K, L, M, N, Q, R, S, T, V, W and Y.

6. The library of claim 1, wherein position 62 is I; position 81 is V; position 87 is L, or position 110 is I; or each polypeptide comprises 162, V71, L87, and I110.

7. (canceled)

8. The library of claim 1, wherein the library is a display library or a yeast two-hybrid library; and/or the library is immobilized to a solid support.

9. The library of claim 1, wherein the library is immobilized to a solid support

10. The library of claim 1, wherein each of the PYR1 receptor polypeptides further comprises a detection reagent.

11. The library of claim 10, wherein the detection agent is a member of a binding complex or an enzymatic complementation reagent

12. A system for detecting a small molecule, comprising a library of claim 1, and a type 2 protein phosphatase (PP2C) polypeptide.

13. The system of claim 12, wherein the PP2C polypeptide is a HAB1 polypeptide.

14. The system of claim 13, wherein the HAB1 polypeptide is an N-terminus deleted HAB1 polypeptide.

15. (canceled)

16. The system of claim 14, wherein the N-terminus deleted HAB1 polypeptide has at least 95% identity to SEQ ID NO:3 and comprises the amino acid residues at positions 393, 406, 411, 416, 455, 467, 481, 494, 499, 549, 565, 584, 629, 646, and 698 as set forth in SEQ ID NO:3; or comprises the amino acid sequence of SEQ ID NO:3.

17. (canceled)

18. The system of claim 12, wherein the PP2C polypeptide is labeled with a detection reagent.

19. The system of claim 12, wherein members of the library are labeled with a first detection reagent and the PP2C polypeptide is labeled with a second detection reagent that interacts with the first detection reagent, wherein a signal is generated when a member of the library binds the small molecule and the first detection reagent interacts with the second detection reagent.

20. The system of claim 19, wherein:
the first detection reagent specifically binds to the second detection reagent, optionally wherein the first detection reagent is an antibody, ligand, or aptamer that specifically binds to the second detection reagent; or the first detection reagent and the second detection reagent are complementing fragments of an enzyme, optionally wherein the enzyme is luciferase; or
the first detection reagent and the second detection reagent are fluorescent moieties, or one of the first detection reagents is a fluorescent moiety and the second is a quenching moiety that quenches the fluorescent moiety; or
the first detection reagent and the second detection reagent are oligonucleotides, wherein the oligonucleotides are proximity assay components.

21.-26. (canceled)

27. The system of claim 20, wherein the proximity assay is a proximity extension assay or proximity ligation assay.

28. A method of identifying a biosensor for a small molecule of interest, comprising contacting the system of claim 12 with a small molecule of interest and identifying that a mutated PYR/PYL contained in the plurality of polypeptides binds to the small molecule of interest.

29. A HAB1 polypeptide comprising an N-terminus deletion, wherein the HAB1 polypeptide has at least 95% identity to SEQ ID NO:3 and comprises the amino acid residues at positions 393, 406, 411, 416, 455, 467, 481, 494, 499, 549, 565, 584, 629, 646, and 698 as set forth in SEQ ID NO:3; or comprises the amino acid sequence of SEQ ID NO:3.

30. (canceled)

31. A cannabinoid-binding polypeptide comprising an amino acid sequence having at least 95% identity to any one of SEQ ID NOS: 6-22, wherein the polypeptide comprises each of the highlighted amino acid residues in the corresponding sequence; or comprises the amino acid sequence of any one of SEQ ID NOS: 6-22; or
an organophosphate-binding polypeptide comprising an amino acid sequence having at least 95% identity to any one of SEQ ID NOS: 23-30, wherein the polypeptide comprises each of the highlighted amino acid residues in the corresponding sequence; or the polypeptide comprises the amino acid sequence of any one of SEQ ID NOS: 23-30; or
a cannabinoid and organophosphate-binding polypeptide comprising an amino acid sequence having at least 95% identity to SEQ ID NO:31 or 32, wherein the polypeptide comprises each of the highlighted amino acid residues in the corresponding sequence; or the polypeptide comprises the amino acid sequence of SEQ ID NO:31 or 32; or
a biosensor polypeptide comprising an amino acid sequence having at least 95% identity to any one of SEQ ID NOS: 33-37, wherein the polypeptide comprises each of the bolded amino acid residues in the corresponding sequence; or the polypeptide comprises the amino acid sequence of any one of SEQ ID NOS: 33-37.

32.-38. (canceled)


SEQUENCE LISTING (183 sequences)

SEQ ID NO: 1 - protein, 191 aa, unidentified
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 2 - protein, 190 aa, synthetic construct
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 3 - protein, 334 aa, synthetic construct
GRSVYELDSIPLWGTVSIQGNASEMEAAFAVVPHFLKLPIKMLMGDHEGMSPSLTHLTGHFFGVYDGHGGSKVADYCRDRLHEALAEEIERIKDELSKRNTGEGRQVQWKKVFTNCFLTVDGEIEGKIGRAVVGSSDKVLEAVASETVGSTAVVALVCSSHIVVANCGDSRAVLFRGKEAIPLSVDHKPDREDEYARIEAAGGKVIQWQGARVFGVLAMSRSIGDRYLKPYVIPEPEVTFMPRSEEDECLILASDGLWDVMSNQEVCEIARRRILMWHKKNGAPPLAERGKGIDPACQAAADYLSKLALQKGSKDNISIIVIDLKAQRKFKTRT

SEQ ID NO: 4 - protein, 718 aa, synthetic construct
MNSSHHHHHHSSGMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGGGGGRSVYELDSIPLWGTVSIQGNASEMEAAFAVVPHFLKLPIKMLMGDHEGMSPSLTHLTGHFFGVYDGHGGSKVADYCRDRLHEALAEEIERIKDELSKRNTGEGRQVQWKKVFTNCFLTVDGEIEGKIGRAVVGSSDKVLEAVASETVGSTAVVALVCSSHIVVANCGDSRAVLFRGKEAIPLSVDHKPDREDEYARIEAAGGKVIQWQGARVFGVLAMSRSIGDRYLKPYVIPEPEVTFMPRSEEDECLILASDGLWDVMSNQEVCEIARRRILMWHKKNGAPPLAERGKGIDPACQAAADYLSKLALQKGSKDNISIIVIDLKAQRKFKTRT

SEQ ID NO: 5 - protein, 511 aa, unidentified
MEEMTPAVAMTLSLAANTMCESSPVEITQLKNVTDAADLLSDSENQSFCNGGTECTMEDVSELEEVGEQDLLKTLSDTRSGSSNVFDEDDVLSVVEDNSAVISEGLLVVDAGSELSLSNTAMEIDNGRVLATAIIVGESSIEQVPTAEVLIAGVNQDTNTSEVVIRLPDENSNHLVKGRSVYELDCIPLWGTVSIQGNRSEMEDAFAVSPHFLKLPIKMLMGDHEGMSPSLTHLTGHFFGVYDGHGGHKVADYCRDRLHFALAEEIERIKDELCKRNTGEGRQVQWDKVFTSCFLTVDGEIEGKIGRAVVGSSDKVLEAVASETVGSTAVVALVCSSHIVVSNCGDSRAVLFRGKEAMPLSVDHKPDREDEYARIENAGGKVIQWQGARVFGVLAMSRSIGDRYLKPYVIPEPEVTFMPRSREDECLILASDGLWDVMNNQEVCEIARRRILMWHKKNGAPPLAERGKGIDPACQAAADYLSMLALQKGSKDNISIIVIDLKAQRKFKTRT

SEQ ID NO: 6 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIWISGLPANTSTERLDILDDERRVTGFSIIGGEHRLTNGKSVTTVHRFEKENRIWTVVLESYVVDMPEGNSEDDTRMAIDTVVKLNLQKLATVAEAMARNSGDGSGSQVTVDSRAL

SEQ ID NO: 7 - protein, 197 aa, synthetic construct
MPSELTPEERSELKNSIAEFHTYQLDPGSCSSLHAQRIHAPPELVWSIVRRFDKPQTYNHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRMLGDTVVKLNLQKLATVAEAMARNSGDGSGSQVTVDSRAL

SEQ ID NO: 8 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDIIVISGLPANTSTERLDILDDERRVTGFSIIGGEHRLTNGKSVTTVHRFEKENRIWTVVLESYVVDMPEGNSEDDTRMLGDTVVKLNLQKLATVAEAMARNSGDGSGSQVTVDSRAL

SEQ ID NO: 9 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNGKSVTTVHRFEKENRIWTVVLESYVVDMPEGNSEDDTRIFGDTVVKLNLQKLATVAEAMARNSGDGSGSQVTVDSRAL

SEQ ID NO: 10 - protein, 197 aa, synthetic construct
MPSGLTPEERSELKNSIAEFHTYQLDPGSCSSLHAQRIHAPPELVWSIVRRFDKPQTYNHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRMLGDTVVKLNLQKLATVAEAMARNSGDGSGSQVTVDSRAL

SEQ ID NO: 11 - protein, 197 aa, synthetic construct
MPSELTPEERSELKNSIAEFHTYQLDPGSCSSLHAQRIHAPPELVWSIVRRFDKPQTFKHFIKSCSVEQNFEMRVGCTRDMIFISGLPANTSTERLDILDDERRVTGFSIIGGEQRLTNGKSVTTVHRFEKENRIWTVVLESYVVDMPEGNSEDDTRMLGDTVVKLNLQKLATVAEAMARNSGGGSGSQVTVDSRAL

SEQ ID NO: 12 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGMPVNTSTERLDILDDKRRVTGFSIIGGEHRLTNGKSVTTVHRFEKENRIWTVVLESYVVDMPEGNSEDDTRMVGDTVVKLNLQKLATVAEAMARNSGDGSGSQVTVDSRAL

SEQ ID NO: 13 - protein, 197 aa, synthetic construct
MPSGLTPEERSELKNSIAEFHTHQLGPGSCSSLHAQRIHAPPELVWSIVRRFDKPQTYQHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAIDTVVKLNLQKLATVAEAMARNSGDGSGSQVTVDSRAL

SEQ ID NO: 14 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEQRLTNGKSVTTVHRFEKENRIWTVVLESYVVDMPEGNSEDDTRMVGDTVVKLNLQKLATVAEAMARNSGDGSGSQVTVDSRAL

SEQ ID NO: 15 - protein, 197 aa, synthetic construct
MPSGLTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEQRLTNGKSVTTVHRFEKENRIWTVVLESYVVDMPEGNSEDDTRMVGDTVVKLNLQKLATVAEAMARNSGDGSGSQVTVDSRAL

SEQ ID NO: 16 - protein, 197 aa, synthetic construct
MPSELTPEERSELKNSIAEFHTYQLDPGSCSSLHAQRIHAPPELVWSIVRRFDKPQTYAHFIKSCSVEQNFEMRVGCTRDVILISGLPANTSTERLDILDDERRVTGFSIIGGEHRLTNYKSVTTVHRFEKENRIWTVVLFSYVVDMPEGNSEDDTRMFADTVVKLNLQKLATVAEAMARNSGDGSGSQVTVDSRAL

SEQ ID NO: 17 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEQRLTNGKSVTTVHRFEKENRIWTVVLESYVVDMPEGNSEDDTRMFADTVWKLNLQKLATVAEAMARNSGDGSGSQATVDSRAL

SEQ ID NO: 18 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEQRLTNGKSVTTVHRFEKENRIWTVVLMSYVVDMPEGNSEDDTRMFADTVWKLNLQKLATVAEAMARNSGDGSGSQVTVDSRAL

SEQ ID NO: 19 - protein, 197 aa, synthetic construct
MPSGLTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEQRLTNGKSVTTVHRFEKENRIWTVVLESYVVDMPEGNSEDDTRMFGDTVVKLNLQKLATVAEAMARNSGDGSGSQVTVDSRAL

SEQ ID NO: 20 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEQRLTNGKSVTTVHRFEKENRIWTVVLESYVVDMPEGNSEDDTRMLGDTVVKLNLQKLATVAEAMARNSGDGSGSQVTVDSRAL

SEQ ID NO: 21 - protein, 197 aa, synthetic construct
MPSGLTPEERSELKNSIAEFHTYQLDPGSCSSLHAQRIRAPPELVWSIVRRFDKPQTYRHLIKSCSVEQNFEMRVGCTRDVIVISGLPANTSTERLDILDDERRVTGFSVIGGDQRLTNGKSVTTVHRFEKENRIWTVVLMSYVVDMPEGNSEDDTRMFADTVVKLNLQKLATVAEAMARNSGDGSGSQVTVDSRAL

SEQ ID NO: 22 - protein, 197 aa, synthetic construct
MPSGLTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEQRLTNGKSVTTVHRFEKENRIWTVVLMSYVVDMPEGNSEDDTRMFADTVWKLNLQKLATVAEAMARNSGDGSGSQVTVDSRAL

SEQ ID NO: 23 - protein, 197 aa, synthetic construct
MPSELTPGERSELKNSIAEFHTYQLDPGSCSSLHAQRIHAPPELVWSIVRRFDKPQTYKHFIKSCSVEQNFEMRVGCTRDYIVISGMPANTSTERLDILDDERRVTGYSIIGGEHRLTNYKSVTTVHRFEKENRIWTVVLESYVVDMPEGNSEDDTRVGVDTVVKLNLQKLATVAEAMARNSGDGSGSQVTVDSRAL

SEQ ID NO: 24 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTMTERLDILDDERRVTGFSIIGGEHRLTSYKQVTTVHRFGKENRIWTVVLESYVVDMPEGNSEDDTRMTTDTVVKLNLQKLATAAEAMARNSGDGSGSQVTVDSRAL

SEQ ID NO: 25 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMFADTWVKLNLQKLATVAEAMARNSGDGSGSQVTVDSRAL

SEQ ID NO: 26 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNLKNVTTVHRFEKENRIWTVVLESYVVDMPEGNSEDDTRMFADTVVKLNLQKLATVAEAMARNSGDGSGSQVTVDSRAL

SEQ ID NO: 27 - protein, 197 aa, synthetic construct
MPSELTPEERSELKNSIAEFHTYQLDPGSCSSLHAQRIHAPPELVWSIVRRFDKPQTYNHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMLADTVVKLNLQKLATVAEAMARNSGDGSGSQVTVDSRAL

SEQ ID NO: 28 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGLSIIGGEHRLTNYKSVTTVHRFEKENRIWTVVLESYVVDMPEGNSEDDTRMLADTVVKLNLQKLATVAEAMARNSGDGSGSQVTVDSRAL

SEQ ID NO: 29 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDLIVISGLPANTSTERLDILDDERRVTGFSIIGGEHRLTNYKSVTTVHRFEKENRIWTVVLESYVVDMPEGNSEDDTRMFADTVVKLGLQKLATVAEAMARNSGDGSGSQVTVDSRAL

SEQ ID NO: 30 - protein, 197 aa, synthetic construct
MPSELTPEERSELKNSIAEFHTYQLDPGSCSSLHAQRIHAPPELVWSIVRRFDKPQTYRHFIKSCSVEQNFEMRAGCTRDVILISGLPANTSTERLDILDDERRVTGYSIIGGEHRLTNYKSVTTVHRFEKENRIWTVVLESYVVDMLEGNSEDDTRMFADTVHKLNLRKLATVAEAMARNSGDGSGSQVTVDSRAL

SEQ ID NO: 31 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMGVDTVVKLNLQKLATVAEAMARNSGDGSGSQVTVDSRAL

SEQ ID NO: 32 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMGIDTVVKLNLQKLATVAEAMARNSGDGSGSQVTVDSRAL

SEQ ID NO: 33 - protein, 190 aa, synthetic construct
MSSSPAVKGLTDEEQKTLEPVIKTYHQFEPDPTTCTSLITQRIHAPASVVWPLIRRFDNPERYRHFIKRCRLISGDGDAGSVREVTLISGLPASTSTERLEFVDDDHRVLSYRIVGGEHRLKNYKSVTSVNEFLNQDSGKVYTVVLESYTVDILEGNTEEDTKMFADTVHKLNLRKLGVAATSAPMHDDE

SEQ ID NO: 34 - protein, 190 aa, synthetic construct
MSSSPAVKGLTDEEQKTLEPVIKTYHQFEPDPTTCTSLITQRIHAPASVVWPLIRRFDNPERYKHFVKRCRLISGDGDVGSVREYTVISGMPASTSTERLEFVDDDHRVLSYRVVGGEHRLKNYKSVTSVNEFLNQDSGKVYTVVLESYTVDIPEGNTEEDTKVGVDTVVKLNLQKLGVAATSAPMHDDE

SEQ ID NO: 35 - protein, 190 aa, synthetic construct
MSSSPAVKGLTDEEQKTLEPVIKTYHQFEPDPTTCTSLITQRIHAPASVVWPLIRRFDNPERYRHFVKRCRLISGDGDVGSVREVTVISGLPASTSTERLEFVDDDHRVLSFRVVGGEQRLKNGKSVTSVNEFLNQDSGKVYTVVLMSYTVDIPEGNTEEDTKMFVDTVWKLNLQKLGVAATSAPMHDDE

SEQ ID NO: 36 - protein, 190 aa, synthetic construct
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEQRLKNGKSVTSVNEFLNQDSGKVYTVVLESYTVDIPEGNTEEDTKMVGDTVVKLNLQKLGVAATSAPMHDDE

SEQ ID NO: 37 - protein, 190 aa, synthetic construct
MSSSPAVKGLTDEEQKTLEPVIKTYHQFEPDPTTCTSLITQRIHAPASVVWPLIRRFDNPERYQHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMAIDTVVKLNLQKLGVAATSAPMHDDE

SEQ ID NO: 38 - DNA, 20 bp, synthetic construct
  source 1..20: mol_type = other DNA
ttggaatttaatcgcggcct

SEQ ID NO: 39 - DNA, 21 bp, synthetic construct
  source 1..21: mol_type = other DNA
ttcaacgggaaacgtcttgct

SEQ ID NO: 40 - DNA, 32 bp, synthetic construct
  source 1..32: mol_type = other DNA
cataatgggatggagtccagctcgtagacgct

SEQ ID NO: 41 - DNA, 32 bp, synthetic construct
  source 1..32: mol_type = other DNA
acgagctggactccatcccattatggggaact

SEQ ID NO: 42 - DNA, 36 bp, synthetic construct
  source 1..36: mol_type = other DNA
acgcttagacaattcatctttaatacgttcaatctc

SEQ ID NO: 43 - DNA, 36 bp, synthetic construct
  source 1..36: mol_type = other DNA
tattaaagatgaattgtctaagcgtaacaccgggga

SEQ ID NO: 44 - DNA, 37 bp, synthetic construct
  source 1..37: mol_type = other DNA
ctcgtatgagaccgtgttacaaccaattaaccaattc

SEQ ID NO: 45 - DNA, 35 bp, synthetic construct
  source 1..35: mol_type = other DNA
ttgaccagatgagaccacggttatccacagaatca

SEQ ID NO: 46 - DNA, 35 bp, synthetic construct
  source 1..35: mol_type = other DNA
ggataaccgtggtctcatctggtcaatcgtacgac

SEQ ID NO: 47 - DNA, 37 bp, synthetic construct
  source 1..37: mol_type = other DNA
tggttgtaacacggtctcatacgagccatagcttcag

SEQ ID NO: 48 - DNA, 30 bp, synthetic construct
  source 1..30: mol_type = other DNA
ataccgcgagatccacgctcaccggctcca

SEQ ID NO: 49 - DNA, 32 bp, synthetic construct
  source 1..32: mol_type = other DNA
tcagcggagatcttttggttttgggagagtag

SEQ ID NO: 50 - DNA, 32 bp, synthetic construct
  source 1..32: mol_type = other DNA
cccaaaaccaaaagatctccgctgactagggc

SEQ ID NO: 51 - DNA, 35 bp, synthetic construct
  source 1..35: mol_type = other DNA
cgtttcactttcggtctcacagacgagttccggag

SEQ ID NO: 52 - DNA, 35 bp, synthetic construct
  source 1..35: mol_type = other DNA
cgtctgtgagaccgaaagtgaaacgtgatttcatg

SEQ ID NO: 53 - DNA, 35 bp, synthetic construct
  source 1..35: mol_type = other DNA
gagttacgtgagacctataaacgcagaaaggccca

SEQ ID NO: 54 - DNA, 35 bp, synthetic construct
  source 1..35: mol_type = other DNA
tgcgtttataggtctcacgtaactccggtgacgga

SEQ ID NO: 55 - DNA, 30 bp, synthetic construct
  source 1..30: mol_type = other DNA
ggtgagcgtggatctcgcggtatcattgca

SEQ ID NO: 56 - DNA, 27 bp, synthetic construct
  source 1..27: mol_type = other DNA
gcgtgttacaaccaattaaccaattct

SEQ ID NO: 57 - DNA, 21 bp, synthetic construct
  source 1..21: mol_type = other DNA
tgaggtctcatacgagccata

SEQ ID NO: 58 - DNA, 20 bp, synthetic construct
  source 1..20: mol_type = other DNA
gtttccctggattgagacag

SEQ ID NO: 59 - DNA, 20 bp, synthetic construct
  source 1..20: mol_type = other DNA
cttgcactgcagaagggatc

SEQ ID NO: 60 - DNA, 20 bp, synthetic construct
  source 1..20: mol_type = other DNA
aatcaagaggtatgtgagat

SEQ ID NO: 61 - DNA, 54 bp, synthetic construct
  source 1..54: mol_type = other DNA
ctgtctcaatccagggaaacgcgtctgagatggaggatgcgtttgcagtagtgc

SEQ ID NO: 62 - DNA, 39 bp, synthetic construct
  source 1..39: mol_type = other DNA
tgctgcctccgtgacccgcgtagactccgaaaaaatggc

SEQ ID NO: 63 - DNA, 39 bp, synthetic construct
  source 1..39: mol_type = other DNA
cgcgggtcacggaggcagcaaagtggcagattactgtcg

SEQ ID NO: 64 - DNA, 20 bp, synthetic construct
  source 1..20: mol_type = other DNA
acaattcgctactacaatgt

SEQ ID NO: 65 - DNA, 30 bp, synthetic construct
  source 1..30: mol_type = other DNA
cgtgacccgcgtagactccgaaaaaatggc

SEQ ID NO: 66 - DNA, 30 bp, synthetic construct
  source 1..30: mol_type = other DNA
tcggagtctacgcgggtcacggaggccaca

SEQ ID NO: 67 - DNA, 51 bp, synthetic construct
  source 1..51: mol_type = other DNA
catatgccttcggagttaacaccaggtgaacgatcggaactaaaaaactca

SEQ ID NO: 68 - DNA, 69 bp, synthetic construct
  source 1..69: mol_type = other DNA
atgcgcgtcggatgcacgcgcgactatatcgtcatcagtggaatgccggcgtctacatcaacggaaaga

SEQ ID NO: 69 - DNA, 57 bp, synthetic construct
  source 1..57: mol_type = other DNA
ggtaactcggaggatgatactcgtgttggtgttgatacggttgtgaagcttaatttg

SEQ ID NO: 70 - DNA, 51 bp, synthetic construct
  source 1..51: mol_type = other DNA
cgattcgacaaaccacaaacataccaaccgttcatcaaatcctgctccgtc

SEQ ID NO: 71 - DNA, 54 bp, synthetic construct
  source 1..54: mol_type = other DNA
aactcggaggatgatactcgtatggctattgatacggttgtgaagcttaatttg

SEQ ID NO: 72 - DNA, 51 bp, synthetic construct
  source 1..51: mol_type = other DNA
cgattcgacaaaccacaaacatacaatccgttcatcaaatcctgctccgtc

SEQ ID NO: 73 - DNA, 27 bp, synthetic construct
  source 1..27: mol_type = other DNA
ctcaggtgacgtgagatccggctgcta

SEQ ID NO: 74 - DNA, 33 bp, synthetic construct
  source 1..33: mol_type = other DNA
ggcatacctcccccgccgccagtctgcgcgtct

SEQ ID NO: 75 - DNA, 33 bp, synthetic construct
  source 1..33: mol_type = other DNA
ggcggcgggggaggtatgccttcggagttaaca

SEQ ID NO: 76 - DNA, 27 bp, synthetic construct
  source 1..27: mol_type = other DNA
cggatctcacgtcacctgagaaccact

SEQ ID NO: 77 - DNA, 20 bp, synthetic construct
  source 1..20: mol_type = other DNA
gtttgtggtttgtcgaatcg

SEQ ID NO: 78 - DNA, 34 bp, synthetic construct
  source 1..34: mol_type = other DNA
cacacatcaactcggtccaggaagctgttcatca

SEQ ID NO: 79 - DNA, 20 bp, synthetic construct
  source 1..20: mol_type = other DNA
cgagtatcatcctccgagtt

SEQ ID NO: 80 - DNA, 36 bp, synthetic construct
  source 1..36: mol_type = other DNA
taccattcgtcagcctttgttcgcctccgatgatac

SEQ ID NO: 81 - DNA, 36 bp, synthetic construct
  source 1..36: mol_type = other DNA
acaaaggctgacgaatggtaaatccgttacgacggt

SEQ ID NO: 82 - DNA, 32 bp, synthetic construct
  source 1..32: mol_type = other DNA
gtatcaccaaccatacgagtatcatcctccga

SEQ ID NO: 83 - DNA, 32 bp, synthetic construct
  source 1..32: mol_type = other DNA
actcgtatggttggtgatacggttgtgaagct

SEQ ID NO: 84 - DNA, 48 bp, synthetic construct
  source 1..48: mol_type = other DNA
cctcatcaagattgctttatgctagctcacgtcacctgagaaccactt

SEQ ID NO: 85 - DNA, 27 bp, synthetic construct
  source 1..27: mol_type = other DNA
gctagcataaagcaatcttgatgagga

SEQ ID NO: 86 - DNA, 51 bp, synthetic construct
  source 1..51: mol_type = other DNA
cagacaagctgtgaccgtcttcccggcgttcagggtaatatattttaaccg

SEQ ID NO: 87 - DNA, 22 bp, synthetic construct
  source 1..22: mol_type = other DNA
gccaggcgcctttatatcatat

SEQ ID NO: 88 - DNA, 42 bp, synthetic construct
  source 1..42: mol_type = other DNA
cgctcatcttgtgcatgagacttgcgaaaccctatgctctgt

SEQ ID NO: 89 - DNA, 46 bp, synthetic construct
  source 1..46: mol_type = other DNA
cggcggaggagtgaccatccgaattcgtcgacgagctcacgcgttc

SEQ ID NO: 90 - DNA, 26 bp, synthetic construct
  source 1..26: mol_type = other DNA
tattaatttagtgtgtgtatttgtgt

SEQ ID NO: 91 - DNA, 49 bp, synthetic construct
  source 1..49: mol_type = other DNA
atacacacactaaattaatagcgcgcatgagtaaaggagaagaactttt

SEQ ID NO: 92 - DNA, 22 bp, synthetic construct
  source 1..22: mol_type = other DNA
tttgtatagttcatccatgcca

SEQ ID NO: 93 - DNA, 46 bp, synthetic construct
  source 1..46: mol_type = other DNA
ggagaaaataccgcatcaggccgcggaatggaaggtcgggatgagc

SEQ ID NO: 94 - DNA, 22 bp, synthetic construct
  source 1..22: mol_type = other DNA
ataaagcagccgctaccaaaca

SEQ ID NO: 95 - DNA, 47 bp, synthetic construct
  source 1..47: mol_type = other DNA
tggtgggggttcatctggtgctagcatgccttcggggttaacaccag

SEQ ID NO: 96 - DNA, 48 bp, synthetic construct
  source 1..48: mol_type = other DNA
ctcatcaagattgctttatgagatcttcacgtcacctgagaaccactt

SEQ ID NO: 97 - DNA, 48 bp, synthetic construct
  source 1..48: mol_type = other DNA
acacaaagatacatacgggaggatccatgccttcggagttaacaccag

SEQ ID NO: 98 - DNA, 49 bp, synthetic construct
  source 1..49: mol_type = other DNA
cctcatcaagattgctttatgctagctcacgtcacctgagaaccacttc

SEQ ID NO: 99 - DNA, 45 bp, synthetic construct
  source 1..45: mol_type = other DNA
actaaagggaacaaaagctggagctccttgccaacagggagttct

SEQ ID NO: 100 - DNA, 47 bp, synthetic construct
  source 1..47: mol_type = other DNA
cttccagagtgaataccatgcggccgcttagattagattgctatgct

SEQ ID NO: 101 - DNA, 51 bp, synthetic construct
  source 1..51: mol_type = other DNA
gacgacgaacggagagttaccggatatagtatcatcggaggcgaacatagg

SEQ ID NO: 102 - DNA, 51 bp, synthetic construct
  source 1..51: mol_type = other DNA
ggaggcgaacataggctgacgaatcataaatccgttacgacggtgcatcgg

SEQ ID NO: 103 - DNA, 51 bp, synthetic construct
  source 1..51: mol_type = other DNA
tcggaggatgatactcgtatgtttggtgatacggttgtgaagcttaatttg

SEQ ID NO: 104 - DNA, 29 bp, synthetic construct
  source 1..29: mol_type = other DNA
acgtacgtgagatccggctgctaacaaag

SEQ ID NO: 105 - DNA, 47 bp, synthetic construct
  source 1..47: mol_type = other DNA
tgctattcatggtatatctccttcttaaagttaaacaaaattatttc

SEQ ID NO: 106 - DNA, 40 bp, synthetic construct
  source 1..40: mol_type = other DNA
gagatataccatgaatagcagccatcatcatcatcatcac

SEQ ID NO: 107 - DNA, 28 bp, synthetic construct
  source 1..28: mol_type = other DNA
cgctgcgacctcccccgccgccagtctg

SEQ ID NO: 108 - DNA, 28 bp, synthetic construct
  source 1..28: mol_type = other DNA
cggcgggggaggtcgcagcgtctacgag

SEQ ID NO: 109 - DNA, 33 bp, synthetic construct
  source 1..33: mol_type = other DNA
cagccggatctcacgtacgtgttttaaacttgc

SEQ ID NO: 110 - DNA, 66 bp, synthetic construct
  source 1..66: mol_type = other DNA
ggaactgtctcaatccagggaaacgcgtctgagatggaggcggcgtttgcagtaagcccccacttt

SEQ ID NO: 111 - DNA, 458 bp, synthetic construct
  source 1..458: mol_type = other DNA
ctgtctcaatccagggaaacgcgtctgagatggaggcggcgtttgcagtagtgccccactttttaaagttacctatcaagatgttgatgggggaccacgaggggatgagtccttctctgacgcacttaacgggccattttttcggagtctacgatggtcacggaggcagcaaagtggcagattactgtcgtgaccgccttcacgaagccctggccgaagagattgaacgtattaaagatgaattgagcaagcgtaacaccggggagggacgccaggtgcagtggaaaaaggtcttcaccaactgtttcctgactgttgatggagagattgaaggaaagatcggtcgcgcagtggtaggtagctccgacaaggtgttggaagcggtcgcgtcagaaactgtggggtcgacagctgtggttgccttagtgtgtagttctcacattgtagtagcgaattgt

SEQ ID NO: 112 - DNA, 457 bp, synthetic construct
  source 1..457: mol_type = other DNA
acattgtagtagcgaattgtggtgattcacgcgcggtgctgtttcgtggcaaggaggctattcccctgagcgttgaccacaaaccggaccgtgaggacgaatatgcccgcatcgaggcggctggtggcaaggtaatccaatggcagggtgcgcgcgtattcggggtcctggcaatgtcacgtagcattggtgaccgctatttgaaaccatacgttatcccggagcctgaggttaccttcatgccccgcagtgaagaagacgaatgtttaattttggccagtgacgggctgtgggatgttatgagcaatcaagaggtatgtgagatcgcccgtcgtcgtatcttaatgtggcacaagaaaaatggggctccacctcttgctgaacgcggcaaagggattgaccccgcttgtcaggctgccgcagactatctgtcaaaacttgcactgcagaagggatc

SEQ ID NO: 113 - DNA, 458 bp, synthetic construct
  source 1..458: mol_type = other DNA
ctgtctcaatccagggaaacgcgtctgagatggaggcggcgtttgcagtagtgccccactttttaaagttacctatcaagatgttgatgggggaccacgaggggatgagtccttctctgacgcacttaacgggccattttttcggagtctacgatggtcacggaggccacaaagtggcagattactgtcgtgaccgccttcactttgccctggccgaagagattgaacgtattaaagatgaattgagcaagcgtaacaccggggagggacgccaggtgcagtgggaaaaggtcttcaccaactgtttcctgactgttgatggagagattgaaggaaagatcggtcgcgcagtggtaggtagctccgacaaggtgttggaagcggtcgcgtcagaaactgtggggtcgacagctgtggttgccttagtgtgtagttctcacattgtagtagcgaattgt

SEQ ID NO: 114 - DNA, 325 bp, synthetic construct
  source 1..325: mol_type = other DNA
acattgtagtagcgaattgtggtgattcacgcgcggtgctgtttcgtggcaaggaggctatgcccctgagcgttgaccacaaaccggaccgtgaggacgaatatgcccgcatcgaggcggctggtggcaaggtaatccaatggcagggtgcgcgcgtattcggggtcctggcaatgtcacgtagcattggtgaccgctatttgaaaccatacgttatcccggagcctgaggttaccttcatgccccgcagtcgcgaagacgaatgtttaattttggccagtgacgggctgtgggatgttatgagcaatcaagaggtatgtgagat

SEQ ID NO: 115 - DNA, 393 bp, synthetic construct
  source 1..393: mol_type = other DNA
atgccaaaaaagaagagaaaggtctcccgaggtggagccgctggaggcactagaccgtatgcctgccctgtagagtcctgtgatagacgttttagcagacatgccaaccttacacgtcacatcagaattcacacagggcaaaaaccttttcagtgcagaatttgtatgagaaatttcagcaggaatgcgaacctagtaagacatattaggacgcacacaggttcccagaagccttttcaatgtcgtatctgtatgcgtaacttctctaggaaggcagacctgagaaggcatatcagaacgcacacaggagagaaaccatttgcgtgcgacatctgcggtaggaagttcgcccgtaaaggcgacttaaaaaggcacacaaagatacatacggga

SEQ ID NO: 116 - DNA, 189 bp, synthetic construct
  source 1..189: mol_type = other DNA
atgccaaaaaagaagagaaaggtctcccgaggtggagccgacgccctggacgatttcgacttggatatgcttggtagtgacgcgctagatgattttgatttggatatgttaggatctgacgccctagacgacttcgatctggatatgcttgggtccgacgcgctggatgatttcgacctggacatgtta

SEQ ID NO: 117 - DNA, 421 bp, synthetic construct
  source 1..421: mol_type = other DNA
gcacaagatgagcggcggaggaggaaaacgtgcggcggaggaggtatagcagcggcggaggagttatcggagcggcggaggagggttctctgtatcatgtcgaagttcgttattggactcattcaacagaagtagcggcggaggagtcactagagcggcggaggagagtgtgttgcggcggaggagagcaactagcggcggaggagcagtgtgcgtaacttccttagctgaagtgctgggaacgctcaatcaaagacgcggcggaggaggctacaaggcggcggaggagtagtgagagcggcggaggagcatacaaggcggcggaggagatcgttggtgcatcatccagtgcggcggaggagatcagcggcggaggagttcagcggcggaggagacaagcggcggaggagtgaccatccgc

SEQ ID NO: 118 - DNA, 110 bp, synthetic construct
  source 1..110: mol_type = other DNA
catagcaatctaatctaagcctaggatggtgaccggctaccggctgttcgaggagattctcgggagttccggtggtggcgggagcggaggtggaggctcgagcggtgcta

SEQ ID NO: 119 - DNA, 548 bp, synthetic construct
  source 1..548: mol_type = other DNA
accgctcgagcctccacctccgctcccgccaccaccggaactcccactgttgatggttactcggaacagcatggagccgtcgggggtgatcaggcgctcgtcgataattttgttgccgttccacagggtccctgttacagtgatctttttgccgtcgaacacggcgatgccttcatacggccgtccgaaatagttcagcatgttcggcgtaaccccgtcgattaccagtgtgccatagggcaggatcaccttaaagtgatgatcatccacagggtacaccaccttaaacacctcttcgatctgggccatttggtcggcgctcagaccttcatacgggatgatgacatggatgtcgatcttcagggcattttcaccgctccggacaatcctttggatcggagttacggacacggcgagattctgcagcaaactggacacacctccctgttcaaggacttggtccaggttgtaggcggctgtctgttcccagtccccaacgaaatcttcgagtgtgaagaccatgcggccgccaccgcggtgatctgtaa

SEQ ID NO: 120 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNGKSVTTVHRFEKENRIWTVVLESYVVDMPEGNSEDDTRMVGDTVVKLNLQKLATVAEAMARNSGDGSGSQVTVDSRAL

SEQ ID NO: 121 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMSLDTVVKLNLQKLATVAEAMARNSGDGSGSQVTVDSRAL

SEQ ID NO: 122 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMSVDTVVKLNLQKLATVAEAMARNSGDGSGSQVTVDSRAL

SEQ ID NO: 123 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMTLDTVVKLNLQKLATVAEAMARNSGDGSGSQVTVDSRAL

SEQ ID NO: 124 - protein, 197 aa, synthetic construct
MPSELTPEERSELKNSIAEFHTYQLDPGSCSSLHAQRIHAPPELVWSIVRRFDKPQTYQHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAIDTVVKLNLQKLATVAEAMARNSGDGSGSQVTVDSRAL

SEQ ID NO: 125 - protein, 197 aa, synthetic construct
MPSELTPEERSELKNSIAEFHTYQLDPGSCSSLHAQRIHAPPELVWSIVRRFDKPQTYQHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAIDTVVKLNLRKLATVAEAMARNSGDGSGSQVTVDSRAL

SEQ ID NO: 126 - protein, 197 aa, synthetic construct
MPSELTPEERSELKNSIAEFHTHQLGPGSCSSLHAQRIHAPPELVWSIVRRFDKPQTYQHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAIDTVVKLNLQKLATVAEAMARNSGDGSGSQVTVDSRAL

SEQ ID NO: 127 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAADTVHKLNLQKLATVAEAMARNSGDGSGSQVTVDSRAL

SEQ ID NO: 128 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNGKSVTTVHRFEKENRIWTVVLESYVVDMPEGNSEDDTRMFGDTVVKLNLQKLATVAEAMARNSGDGSGSQVTVDSRAL

SEQ ID NO: 129 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRMFGDTVVKLNLQKLATVAEAMARNSGDGSGSQVTVDSRAL

SEQ ID NO: 130 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMFGDTVVKLNLQKLATVAEAMARNSGDGSGSQVTVDSRAL

SEQ ID NO: 131 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMAIDTVVKLNLQKLATVAEAMARNSGDGSGSQVTVDSRAL

SEQ ID NO: 132 - protein, 197 aa, synthetic construct
MPSELTPEERSELKNSIAEFHTYQLDPGSCSSLHAQRIHAPPELVWSIVRRFDKPQTYNHFIKSCSVEQNFEMRVGCTRDVIVISGLPANTSTERLDILDDERRVTGFSIIGGEHRLTNHKSVTTVHRFEKENRIWTVVLESYVVDMPEGNSEDDTRMFGDTVVKLNLQKLATVAEAMARNSGDGSGSQVTVDSRAL

SEQ ID NO: 133 - protein, 197 aa, synthetic construct
MPSELTPEERSELKNSIAEFHTYQLDPGSCSSLHAQRIHAPPELVWSIVRRFDKPQTYTHFIKSCSVEQNFEMRVGCTRDVIVISGLPANTSTERLDILDDERRVTGFSIIGGEHRLTNGKSVTTVHRFEKENRIWTVVLESYVVDMPEGNSEDDTRMFGDTVVKLNLQKLATVAEAMARNSGDGSGSQVTVDSRAL

SEQ ID NO: 134 - protein, 197 aa, synthetic construct
MPSELTPEERSELKNSIAEFHTYQLDPGSCSSLHAQRIHAPPELVWSIVRRFDKPQTYSHFIKSCSVEQNFEMRVGCTRDVIWISGLPANTSTERLDILDDERRVTGFSIIGGEHRLTNGKSVTTVHRFEKENRIWTVVLESYVVDMPEGNSEDDTRMAADTVVKLNLQKLATVAEAMARNSGDGSGSQVTVDSRAL

SEQ ID NO: 135 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDIIVISGLPANTSTERLDILDDERRVTGFSIIGGEHRLTNGKSVTTVHRFEKENRIWTVVLESYVVDMPEGNSEDDTRMFGDTVVKLNLQKLATVAEAMARNSGDGSGSQVTVDSRAL

SEQ ID NO: 136 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNGKSVTTVHRFEKENRIWTVVLESYVVDMPEGNSEDDTRMLGDTVVKLNLQKLATVAEAMARNSGDGSGSQVTVDSRAL

SEQ ID NO: 137 - protein, 197 aa, synthetic construct
MPSELTPEERSELKNSIAEFHTYQLDPGSCSSLHAQRIHAPPELVWSIVRRFDKPQTYSHFIKSCSVEQNFEMRVGCTRDVIWISGLPANTSTERLDILDDERRVTGFSIIGGEQRLTNGKSVTTVHRFEKENRIWTVVLESYVVDMPEGNSEDDTRMLGDTVVKLNLQKLATVAEAMARNSGDGSGSQVTVDSRAL

SEQ ID NO: 138 - protein, 197 aa, synthetic construct
MPSGLTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEQRLTNGKSVTTVHRFEKENRIWTVVLMSYVVDMPEGNSEDDTRMFADTVVKLNLQKLATVAEAMARNSGDGSGSQVTVDSRAL

SEQ ID NO: 139 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGPPANTSTERLDILDDERRVTGFSIIGGEHRLTNAKSVTTVHRFEKENRIWTVVLESYVVDMPEGNSEDDTRMFADTVVKLNLQKLATVAEAMARNSGDGSGSQVTVDSRAL

SEQ ID NO: 140 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGMPANTSTERLDILDDERRVTGFSIIGGEHRLTNYKSVTTVHRFEKENRIWTVVLESYVVDMPEGNSEDDTRMGADTVVKLNLQKLATVAEAMARNSGDGSGSQVTVDSRAL

SEQ ID NO: 141 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGYSIIGGEHRLTNYKSVTTVHRFEKENRIWTVVLESYVVDMPEGNSEDDTRMGADTVVKLNLQKLATVAEAMARNSGDGSGSQVTVDSRAL

SEQ ID NO: 142 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMLIDTVVKLNLQKLATVAEAMARNSGDGSGSQVTVDSRAL

SEQ ID NO: 143 - protein, 197 aa, synthetic construct
MPSELTPEERSELKNSIAEFHTYQLDPGSCSSLHAQRIHAPPELVWSIVRRFDKPQTYMHFIKSCSVEQNFEMRVGCTRDVIMISGLPANTSTERLDILDDERRVTGFSIIGGEHRLTNAKSVTTVHRFEKENRIWTVVLESYVVDMPEGNSEDDTRMFADTVVKLNLQKLATVAEAMARNSGDGSGSQVTVDSRAL

SEQ ID NO: 144 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIMISGLPANTSTERLDILDDERRVTGFSIIGGEHRLTNAKSVTTVHRFEKENRIWTVVLESYVVDMPEGNSEDDTRMFADTVVKLNLQKLATVAEAMARNSGDGSGSQVTVDSRAL

SEQ ID NO: 145 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGYSIIGGEHRLTNAKSVTTVHRFEKENRIWTVVLESYVVDMPEGNSEDDTRMFADTVVKLNLQKLATVAEAMARNSGDGSGSQVTVDSRAL

SEQ ID NO: 146 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMFADTVVKLNLQKLATVAEAMARNSGDGSGSQVTVDSRAL

SEQ ID NO: 147 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGYSIIGGEHRLTNYKSVTTVHRFEKENRIWTVVLESYVVDMPEGNSEGDTRMGVDTVVKLNLQKLATVAEAMARNSGDGSGSQVTVDSRAL

SEQ ID NO: 148 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGMPANTSTERLDILDDERRVTGYSIIGGEHRLTNYKSVTTVHRFEKENRIWTVVLESYVVDMPEGNSEDDTRMGVDTVVKLNLQKLATVAEAMARNSGDGSGSQVTVDSRAL

SEQ ID NO: 149 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDYIVISGMPANTSTERLDILDDERRVTGYSIIGGEHRLTNYKSVTTVHRFEKENRIWTVVLESYVVDMPEGNSEDDTRMGVDTVVKLNLQKLATVAEAMARNSGDGSGSQVTVDSRAL

SEQ ID NO: 150 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMLADTVVKLNLQKLATVAEAMARNSGDGSGSQVTVDSRAL

SEQ ID NO: 151 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMTADTVVKLNLQKLATVAEAMARNSGDGSGSQVTVDSRAL

SEQ ID NO: 152 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMIADTVIKLNLQKLATVAEAMARNSGDGSGSQVTVDSRAL

SEQ ID NO: 153 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMTADTHVKLNLQKLATVAEAMARNSGDGSGSQVTVDSRAL

SEQ ID NO: 154 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMINDTVVKLNLQKLATVAEAMARNSGDGSGSQVTVDSRAL

SEQ ID NO: 155 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNAKSVTTVHRFEKENRIWTVVLESYVVDMPEGNSEDDTRMAADTVVKLNLQKLATVAEAMARNSGDGSGSQVTVDSRAL

SEQ ID NO: 156 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMLADTVVKLNLQKLATVAEAMARNSGDGSGSQVTVDSRAL

SEQ ID NO: 157 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSAIGGEHRLTNYKSVTTVHRFEKENRIWTVVLESYVVDMPEGNSEDDTRMIADTVVKLNLQKLATVAEAMARNSGDGSGSQVTVDSRAL

SEQ ID NO: 158 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMTADTVVKLNLQKLATVAEAMARNSGDGSGSQVTVDSRAL

SEQ ID NO: 159 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMCADTVVKLNLQKLATVAEAMARNSGDGSGSQVTVDSRAL

SEQ ID NO: 160 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMIADTVVKLNLQKLATVAEAMARNSGDGSGSQVTVDSRAL

SEQ ID NO: 161 - protein, 197 aa, synthetic construct
MPSELTPEERSELKNPIAEFHTYQLDPGGCSSLHARRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMTADTVVKLNLQKLATVAEAMARNSGDGSGSQVTVDSRAL

SEQ ID NO: 162 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTMTERLDILDDERRVTGFSIIGGEHRLTNYKSVTTVHRFEKENRIWTVVLESYVVDMPEGNSEDDTRMTADTVVKLNLQKLATAAEAMARNSGDGSGSQVTVDSRAL

SEQ ID NO: 163 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTMTERLDILDDERRVTGFSIIGGEHRLTNYKQVTTVHRFGKENRIWTVVLESYVVDMPEGNSEDDTRMTADTVVKLNLQKLATAAEAMARNSGDGSGSQVTVDSRAL

SEQ ID NO: 164 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGASIIGGEHRLTNYKSVTTVHRFEKENRIWTVVLESYVVDMPEGNSEDDTRMFADTVVKLNLQKLATVAEAMARNSGDGSGSQVTVDSRAL

SEQ ID NO: 165 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSAIGGEHRLTNYKSVTTVHRFEKENRIWTVVLESYVVDMPEGNSEDDTRMGADTVVKLNLQKLATVAEAMARNSGDGSGSQVTVDSRAL

SEQ ID NO: 166 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGLSIIGGEHRLTNYKSVTTVHRFEKENRIWTVVLESYVVDMPEGNSEDDTRMLADTVVKLNLQKLATVAEAMARNSGDGSGSQVTVDSRAL

SEQ ID NO: 167 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSAIGGEHRLTNYKSVTTVHRFEKENRIWTVVLESYVVDMPEGNSEDDTRMLADTVVKLILQKLATVAEAMARNSGDGSGSQVTVDSRAL

SEQ ID NO: 168 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIIISGLPANTSTERLDILDDERRVTGFSIIGGEHRLTNYKSVTTVHRFEKENRIWTVVLESYVVDMPEGNSEDDTRMLADTVVKLNLQKLATVAEAMARNSGDGSGSQVTVDSRAL

SEQ ID NO: 169 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRRWTVVLESYVVDMPEGNSEDDTRMFADWVVKLNLQKLATVAEAMARNSGDGSGSQVTVDSRAL

SEQ ID NO: 170 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLGLQKLATVAEAMARNSGDGSGSQVTVDSRAL

SEQ ID NO: 171 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRTFADTVVKLNLQKLATVAEAMARNSGDGSGSQVTVDSRAL

SEQ ID NO: 172 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMHADTVVKLGLQKLATVAEAMARNSGDGSGSQVTVDSRAL

SEQ ID NO: 173 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKGVTTVHRFEKENRIWTVVLESYVVDMPEGNSEDDTRMFADTVVKLGLQKLATVAEAMARNSGDGSGSQVTVDSRAL

SEQ ID NO: 174 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGWSIIGGEHRLTNYKSVTTVHRFEKENRIWTVVLESYVVDMPEGNSEDDTRMFADTVVKLGLQKLATVAEAMARNSGDGSGSQVTVDSRAL

SEQ ID NO: 175 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGIPANTSTERLDILDDERRVTGFSIIGGEHRLTNYKSVTTVHRFEKENRIWTVVLESYVVDMPEGNSEDDTRMFADTVVKLGLQKLATVAEAMARNSGDGSGSQVTVDSRAL

SEQ ID NO: 176 - protein, 197 aa, synthetic construct
MPSELTPEERSELKNSIAEFHTYQLDPGSCSSLHAQRIHAPPELVWSIVRRFDKPQTYRHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRVFADTVVKLNLQKLATVAEAMARNSGDGSGSQVTVDSRAL

SEQ ID NO: 177 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMALDTVVKLNLQKLATVAEAMARNSGDGSGSQVTVDSRAL

SEQ ID NO: 178 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGMPANTSTERLDILDDERRVTGFSIIGGEHRLTNHKSVTTVHRFEKENRIWTVVLESYVVDMPEGNSEDDTRMFADTVVKLNLQKLATVAEAMARNSGDGSGSQVTVDSRAL

SEQ ID NO: 179 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIVISGLPANTSTDRLDILDDERRVTGFSIIGGEHRLTNYKSVTTVHRFEKENRIWTVVLESYVVDMPEGNSEDDTRMFADTVHKLNLQKLATVAEAMARNSGDGSGSQVTVDSRAL

SEQ ID NO: 180 - protein, 197 aa, synthetic construct
MPSELTPEERSELKNSIAEFHTYQLDPGSCSSLHAQRIHAPPELVWSIVRRFDKPQTYKHFIKSCSVEQNFEMRVGCTRDVIAISGLPANTSTDRLDILDDERRVTGFSIIGGEHRLTNYKSVTTVHRFEKENRIWTVVLESYVVDMPEGNSEDDTRMFADTVHKLNLQKLATVAEAMARNSGGGSGSQVTVDSRAL

SEQ ID NO: 181 - protein, 197 aa, synthetic construct
MPSELTPEERSELKNSIAEFHTYQLDPGSCSSLHAQRIHAPPELVWSIVRRFDKPQTYRHFIKSCSVEQNFEMRAGCTRDVILISGLPANTSTERLDILDDERRVTGFSIIGGEHRLTNYKSVTTVHRFEKENRIWTVVLESYVVDMLEGNSEDDTRMFADTVVHLNLQKLATVAEAMARNSGDGSGSQVTVDSRAL

SEQ ID NO: 182 - protein, 197 aa, synthetic construct
MPSELTPEERSELKNSIAEFHTYQLDPGSCSSLHAQRIHAPPELVWSIVRRFDKPQTYRHFIKSCSVEQNFEMRAGCTRDVILISGLPANTSTERLDILDDERRVTGFSIIGGEHRLTNYKSVTTVHRFEKENRIWTVVLESYVVDMLEGNSEDDTRMFADTVVHLNLQKLATVAEAMARNSGGGSGSQVTVDSRAL

SEQ ID NO: 183 - protein, 197 aa, synthetic construct
MPSELAPEERSELKNSIAEFHTYQLDPGSCSSLHAQRIHAPPELVWSIVRRFDKPQTYRHFIKSCSVEQNFEMRAGCTRDVILISGLPANTSTERLDILDDERRVTGYSIIGGEHRLTNYKSVTTVHRFEKENRIWTVVLESYVVDMLEGNSEDDTRMFADTVVHLNLQKLATVAEAMARNSGDGSGSQVTVDSRAL